(19) United States
(12) Patent Application Publication
(10) Pub. No.: US 2015/0258108 A1
(43) Pub. Date: Sep. 17, 2015

(54) METHODS AND COMPOSITIONS FOR REDUCING TOLERANCE TO OPIOID ANALGESICS

(71) Applicant: DemeRx, Inc., Fort Lauderdale, FL (US)

(72) Inventors: Emeline Maillet, Miami Beach, FL (US); Lawrence Friedhoff, River Vale, NJ (US)

(21) Appl. No.: 14/624,434

(22) Filed: Feb. 17, 2015

Related U.S. Application Data
(60) Provisional application No. 61/952,741, filed on Mar. 13, 2014.
(60) Provisional application No. 62/005,841, filed on May 30, 2014.

(51) Int. Cl. A61K 31/55

(52) U.S. Cl. CPC A61K 31/55

(57) ABSTRACT

A method for modulating tolerance to an opioid analgesic in a patient undergoing opioid analgesic therapy, the method comprising interrupting or administering concurrently with said opioid analgesic therapy an amount of noribogaine, noribogaine derivative, or pharmaceutically acceptable salt and/or solvate thereof that provides a therapeutic serum concentration of noribogaine. In some embodiments, the therapeutic average serum concentration is 50 ng/mL to 180 ng/mL, said concentration being sufficient to re-sensitize the patient to the opioid as an analgesic while maintaining a QT interval of less than about 500 ms during said treatment.

### CROSS-REFERENCE TO RELATED APPLICATIONS

This application claims priority to U.S. Provisional Application No. 62/005,841, filed May 30, 2014 and 61/952,741, filed Mar. 31, 2014, which are incorporated herein by reference in their entirety.

### FIELD OF THE INVENTION

This invention is directed to methods for reducing tolerance to opioids in a patient undergoing opioid analgesic treatment for pain comprising treating the patient with noribogaine, noribogaine derivative, or pharmaceutically acceptable salt or solvate thereof at a dosage that provides a therapeutic serum concentration. In one embodiment, the average serum concentration is 50 ng/mL to 180 ng/mL, including under conditions where the QT interval prolongation does not exceed about 50 milliseconds, and preferably 30 milliseconds.

### STATE OF THE ART

Addictive opioid analgesic agents such as morphine are well-known and exceptionally potent analgesics. Such opioids operate as mu receptor agonists. Upon administration, opioids initiate a cascade of biological events including increased serotonin and dopamine expression. As is well known, continued use of many such opioids (especially at high doses) carries a significant risk of dependency/addiction. Indeed, potential addiction to such opioids is a serious issue that limits the therapeutic use of addictive opioids as analgesic agents. For example, the use of morphine as an analgesic is common among end stage patients suffering from serious pain where addiction is no longer a concern.

Drug tolerance to opioid analgesics is common, and may be psychological and/or physiological. A patient who has developed tolerance to the opioid analgesic is not necessarily addicted to or misusing the analgesic. Drug tolerance occurs when the patient's reaction to the drug is reduced, requiring an increase in dose to achieve the same desired effect. There are several potential methods for how tolerance develops, including receptor desensitization, receptor phosphorylation, receptor internalization or down-regulation, and up-regulation of inhibitory pathways.

Drug tolerance requires that the dosage of analgesic be increased in order to provide sustained analgesic effect. However, high doses of opioids may lead to serious complications and side effects, including physical dependence, addiction, respiratory depression, nausea, sedation, euphoria or dysphoria, decreased gastrointestinal motility, and itching.

It would be beneficial to provide a method for modulating opioid analgesic tolerance in a patient taking one or more opioid analgesics for the treatment of pain.

### SUMMARY OF THE INVENTION

This invention is directed, in part, to the use of noribogaine to modulate tolerance to addictive opioid analgesic agents in a patient who has developed or is at risk of developing a tolerance for the analgesic. In such methods, effective analgesia can be achieved in a patient while resensitizing the patient to the addictive opioid analgesic. The term “resensitizing the patient” is used herein to refer to reducing, relieving, attenuating, and/or reversing tolerance to the analgesic. In one aspect, the resensitized patient obtains therapeutic effect from a lower dose of the opioid analgesic than before resensitization. In one aspect, the resensitized patient obtains improved therapeutic effect from the same dose of the opioid analgesic compared to before resensitization.

It has been discovered that the use of noribogaine imparts a dose dependent prolongation of the treated patient's QT interval, rendering higher dosing of noribogaine unacceptable. A prolonged QT interval is a marker of potential Torsades de Pointes, a serious arrhythmia that can result in death.

The current invention is predicated on the surprising discovery that treatment with a narrow dosage range of noribogaine, noribogaine derivative, or pharmaceutically acceptable salt and/or solvate thereof, between 1 mg/kg body weight and 4 mg/kg body weight, provides a therapeutic modulation of in tolerance to opioid analgesics. Preferably, the dose range that provide both therapeutic results and an acceptable QT interval prolongation of less than 50 milliseconds is between 1.3 mg per kg body weight and no more than 4 mg per kg body weight and, more preferably between 1.3 mg per kg body weight and no more than 3 mg per kg body weight, or any subrange or subvalue within the aforementioned ranges.

In a preferred embodiment, the narrow therapeutic doses of noribogaine, noribogaine derivative, or pharmaceutically acceptable salt and/or solvate described above do not prolong the QT interval to unacceptable levels in human patients. In some embodiments, patients will be administered therapeutic doses of noribogaine, noribogaine derivative, or pharmaceutically acceptable salt and/or solvate thereof in a clinical setting with cardiac monitoring. In some embodiments, the patient will be pre-screened to evaluate tolerance for prolongation of QT interval, e.g., to determine whether the patient has any pre-existing cardiac conditions which would disqualify them from treatment with noribogaine.

In one embodiment, noribogaine, noribogaine derivative, or pharmaceutically acceptable salt and/or solvate thereof is administered concurrently with the opioid analgesic. In one embodiment, noribogaine, noribogaine derivative, or pharmaceutically acceptable salt and/or solvate thereof is administered after administration of the analgesic, for example one, two, three, four, eight, ten, twelve, 24 hours or more after administration of the analgesic. In one embodiment, one dose of noribogaine is administered. In one embodiment, two or more doses of noribogaine are administered. In one embodiment, the opioid analgesic is interrupted for a period of time while noribogaine is administered. In one embodiment, a non-opioid analgesic is administered while the opioid analgesic is interrupted. In one embodiment, noribogaine acts as an analgesic. In one embodiment, the opioid analgesic is not interrupted during noribogaine treatment.

In some embodiments, the therapeutic dose of noribogaine, noribogaine derivative, or pharmaceutically acceptable salt and/or solvate thereof administered to the patient is sufficient to provide an average serum concentration of 50 ng/mL to 180 ng/mL (area under the curve/24 hours), or any subrange or subvalue there between. In a preferred embodiment, the dose of noribogaine, noribogaine derivative, or pharmaceutically acceptable salt and/or solvate thereof administered to the patient provides an average serum concentration of 80 ng/mL to 100 ng/mL.

In some embodiments, the dose of noribogaine, noribogaine derivative, or pharmaceutically acceptable salt and/or solvate thereof that provides an average serum concentration of 50 ng/mL to 180 ng/mL is administered as a single dose. In some embodiments, the dose of noribogaine, noribogaine derivative, or pharmaceutically acceptable salt and/or solvate thereof that provides an average serum concentration of 50 ng/mL to 180 ng/mL is administered as multiple doses. In one embodiment, the aggregate dose of noribogaine, noribogaine derivative, or pharmaceutically acceptable salt and/or solvate thereof is from 1 mg/kg to 4 mg/kg. In one embodiment, the aggregate dose of noribogaine, noribogaine derivative, or pharmaceutically acceptable salt and/or solvate thereof is from 1 mg/kg to 3 mg/kg. In one embodiment, the aggregate dose of noribogaine, noribogaine derivative, or pharmaceutically acceptable salt and/or solvate thereof is from 1.3 mg/kg to 2 mg/kg. In one embodiment, the aggregate dose of noribogaine, noribogaine derivative, or pharmaceutically acceptable salt and/or solvate thereof is about 2 mg/kg.

In some embodiments, the unit dose that provides both therapeutic results and an acceptable QT interval prolongation of less than 50 milliseconds in opioid-tolerant humans is about 120 mg. In some embodiments, the unit dose that provides both therapeutic results and an acceptable QT interval prolongation of less than 50 milliseconds in opioid-tolerant humans is 2 mg/kg body weight.

In some embodiments, the patient is administered an initial dose of noribogaine, noribogaine derivative, or pharmaceutically acceptable salt or solvate thereof, followed by one or more additional doses. In one embodiment, the initial dose is from 75 mg to 120 mg. In one embodiment, the one or more additional doses are lower than the initial dose. In one embodiment, the one or more additional doses are from 5 mg to 50 mg. In one embodiment, such a dosing regimen provides an average serum concentration of noribogaine of 50 ng/mL to 180 ng/mL. In one embodiment, the one or more additional doses maintain an average serum concentration of 50 ng/mL to 180 ng/mL over a period of time. In one embodiment, the one or more additional doses are administered periodically.

In some embodiments, the serum concentration is sufficient to modulate said tolerance while maintaining a QT interval of less than 500 milliseconds (ms) during said treatment. In some embodiments, the therapeutic dose of noribogaine, noribogaine derivative, or pharmaceutically acceptable salt and/or solvate thereof provides prolongation of the QT interval of less than 80 ms. In one embodiment, the dose of noribogaine, noribogaine derivative, or pharmaceutically acceptable salt and/or solvate thereof provides prolongation of the QT interval of less than 50 ms. In some embodiments, the dose of noribogaine, noribogaine derivative, or pharmaceutically acceptable salt and/or solvate thereof provides prolongation of the QT interval of less than 30 ms. In a preferred embodiment, the dose of noribogaine, noribogaine derivative, or pharmaceutically acceptable salt and/or solvate thereof provides prolongation of the QT interval of less than 20 ms. In one embodiment, the patient is tested to determine QT interval and/or risk of prolongation before treatment with noribogaine, and if clinician determines that the QT prolongation would be unacceptable risk, noribogaine therapy will be contraindicated.

### BRIEF DESCRIPTION OF THE FIGURES

FIG. 1 represents mean noribogaine concentration-time profiles in healthy patients after single oral dosing with 3, 10, 30 or 60 mg doses. Inset: Individual concentration-time profiles from 0-12 h after a 10 mg dose.

FIG. 2 represents mean plasma noribogaine glucuronide concentration-time profiles in healthy patients after single oral 30 or 60 mg doses.

FIG. 3 illustrates the mean noribogaine concentration-time profile in opioid-addicted patients after a single oral 60 mg (diamonds), 120 mg (squares), or 180 mg (triangles) dose of noribogaine.

FIG. 4 illustrates hours to resumption of opioid substitution treatment (OST) for each patient given placebo (circles), or a single oral dose of noribogaine (60 mg, squares; 120 mg, triangles; 180 mg, inverted triangles). Center horizontal line represents mean. Error bars represent standard deviation.

FIG. 5 illustrates results of noribogaine treatment on final COWS scores before resumption of OST. Boxes include values representing 25%-75% quartiles. Diamonds represent the median, crossbars represent mean. Whiskers represent values within one standard deviation of mid-quartiles. No outliers were present.

FIG. 6A illustrates of the mean change in total COWS scores over the first 6 hours following dosing of noribogaine (60 mg, squares; 120 mg, triangles; 180 mg, diamonds) or placebo (circles). Data is given relative to baseline COWS score.

FIG. 6B illustrates the mean area under the curve (AUC) over the initial 6 hour period after administration of noribogaine or placebo, based on the COWS score data given in FIG. 6A. A negative change in score indicates that withdrawal symptoms subsided over the period.

FIG. 7A illustrates of the mean change in total OOWS scores over the first 6 hours following dosing of noribogaine (60 mg, squares; 120 mg, triangles; 180 mg, diamonds) or placebo (circles). Data is given relative to baseline OOWS score.

FIG. 7B illustrates the mean area under the curve (AUC) over the initial 6 hour period after administration of noribogaine or placebo, based on the OOWS score data given in FIG. 7A. A negative change in score indicates that withdrawal symptoms subsided over the period.

FIG. 8A illustrates of the mean change in total SOWS scores over the first 6 hours following dosing of noribogaine (60 mg, squares; 120 mg, triangles; 180 mg, diamonds) or placebo (circles). Data is given relative to baseline SOWS score.

FIG. 8B illustrates the mean area under the curve (AUC) over the initial 6 hour period after administration of noribogaine or placebo, based on the SOWS score data given in FIG. 8A. A negative change in score indicates that withdrawal symptoms subsided over the period.

FIG. 9A illustrates the average change in QT interval (ΔQTcl) for each cohort (60 mg, squares; 120 mg, triangles; 180 mg, diamonds) or placebo (circles) over the first 24 hours post administration.

FIG. 9B illustrates the correlation between serum noribogaine concentration and ΔQTcl for each patient over time. The equation of the line is given.

FIG. 10 illustrates affinity of noribogaine at the mu (OPRM) and kappa (OPRK) opioid receptors. Competitive inhibition by noribogaine and ibogaine of [³H]-DAMGO binding to (A) OPRM and of [³H]-U69,593 binding to (B) OPRK was performed in CHO—K1 cells membrane preparation. Data used for the non-linear regression analysis are shown as the mean±SEM of each representative experiment(s). Mean±SEM of apparent binding affinity Kᵢ values of a least 3 experiments are shown in Table 5.

FIG. 11 illustrates noribogaine-induced stimulation of [35S]GTPγS binding at the mu (OPRM) and kappa (OPRK) opioid receptors. CHO—K1 cells membrane preparation expressing the OPRM (FIG. 11A) or the OPRK (FIG. 11B) receptors were stimulated by increasing concentrations of agonists (DAMGO, Morphine: MOR, Nalmefene: NALM) and test compounds (Noribogaine: NORI, Ibogaine: IBO; 18-MC). Data used for the non-linear regression analysis are shown as the mean±SEM of each representative experiment(s). Mean±SEM of EC₅₀ and Eₘₐₓ values of 2 to 10 experiments are shown in Table 6.

FIG. 12 illustrates that noribogaine inhibits agonist-induced [³⁵S]GTPγS binding at the mu receptor (OPRM). CHO—K1cells membrane preparation expressing the OPRM receptors were stimulated by increasing concentration of agonists DAMGO and Morphine (MOR) in the presence of 150 μM Noribogaine as shown in FIG. 12A. [³⁵S]GTPγS binding signal from 4 concentrations of DAMGO (3, 30, 90, and 270 nM) was recorded in the presence of increasing concentrations of noribogaine as shown in FIG. 12B. Data used for the non-linear regression analysis are shown as the mean±SEM of each representative experiment(s) performed in triplicate.

FIG. 13 illustrates that noribogaine partially inhibits of agonist-induced [35S]GTPγS binding at the kappa receptor (OPRK). Experiments tested the effects of the partial agonists (FIG. 13A, 13B) Nalmefene (NALM) and (FIG. 13C, 13D) Noribogaine (NORI) in the presence of other agonists. (FIG. 13A, 13C) CHO—K1 cells membrane preparation expressing the OPRK receptors were stimulated by increasing concentration of agonists Dynorphin A (left panel-DYNA) or Morphine (right panel-MOR) in the presence of 3 nM Nalmefene or 150 μM Noribogaine at ˜5× their EC₅₀. Controls antagonists (FIG. 13A) NorBNI and (FIG. 13C) 18-MC were added at 5 nM and 100 μM in similar conditions and compared for right-shift of the agonists dose-responses. (FIG. 13B, 13D) Membranes were stimulated by increasing concentrations of (FIG. 13B) Nalmefene or (FIG. 13D) Noribogaine in the presence of agonists U69,593 (100 nM), Morphine (MOR-6 and 5 μM), Noribogaine (NORI-10 and 100 μM) and

Nalmefene (NALM-20 nM). Data are shown as the mean±SEM of representative experiment(s) performed in triplicate.

FIG. 14 illustrates that noribogaine inhibits agonist-induced β-arrestin recruitment at the mu (OPRM) and kappa (OPRK) receptors. CHO—K1 cells expressing the OPRM receptors were stimulated by increasing concentrations of the reference agonist [Met]-Enkephalin (MET-K) and test compound Noribogaine (NORI) as shown in FIG. 14A. CHO—K1 cells expressing the OPRK receptors were stimulated by increasing concentrations of the reference agonist Dynorphin A (DYNA) and test compound Noribogaine (NORI) as shown in FIG. 14B. Reference agonists were applied at a concentration of 80% their EC₅₀ in the presence of increasing concentrations of noribogaine. Data used for the non-linear regression analysis are shown as the mean±SEM of one standardized experiment performed in duplicate as shown in FIGS. 14A & 14B.

FIG. 15 illustrates that ligand-protein binding contacts of Noribogaine with OPMR over a 12 ns molecular dynamics simulation; data shown are the prevalent interactions that occur more than 30% in the simulation time.

FIG. 16 illustrates binding model of Noribogaine in OPMR extracted from a molecular dynamics simulation.

FIG. 17 illustrates inhibition of Noribogaine-induced [35S]GTPγS binding by OPRK antagonists. Noribogaine dose-response curves were examined in the presence of increasing concentrations of Nalmefene (NALM) as shown in FIG. 17A. The inhibitory effects of antagonists Naloxone (NALO-30 nM), Nalmefene (NALM-3 nM) and NorBNI (5 nM) were tested against increasing concentrations of Noribogaine, and control agonists U69,593, Dynorphin A, Morphine, and Nalmefene-induced signal as shown in FIG. 17B-17E. Functional inhibition constant Ke was extracted for each dose response-curve shifts and Table 7 represents the mean±SEM of 3 up to 7 experiments. (FIG. 17A, FIG. 17B-NALM) and Noribogaine (FIG. 17C, 17D-NORI) in the presence of other agonists. Data used for the analysis are shown as the mean±SEM of representative experiment(s).

### DETAILED DESCRIPTION OF THE INVENTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

### I. Definitions

Unless defined otherwise, all technical and scientific terms used herein have the same meaning as commonly understood by one of ordinary skill in the art to which this invention belongs. As used herein the following terms have the following meanings

The term “about” when used before a numerical designation, e.g., temperature, time, amount, concentration, and such other, including a range, indicates approximations which may vary by (+) or (−) 20%, 10%, 5%, 1%, or any subrange or subvalue there between. Preferably, the term “about” when used with regard to a dose amount means that the dose may vary by +/−20%. For example, “about 2 mg/kg noribogaine” indicates that a patient may be administered a dose of noribogaine between 1.6 mg/kg and 2.4 mg/kg. In another example, about 120 mg per unit dose of noribogaine indicates that the unit dose may range from 96 mg to 144 mg.

“Administration” refers to introducing an agent, such as noribogaine, into a patient. Typically, an effective amount is administered, which amount can be determined by the treating physician or the like. Any route of administration, such as oral, topical, subcutaneous, peritoneal, intra-arterial, inhalation, vaginal, rectal, nasal, introduction into the cerebrospinal fluid, or instillation into body compartments can be used. The agent, such as noribogaine may be administered by direct blood stream delivery, e.g. sublingual, buccal, intranasal, or intrapulmonary administration.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of an agent, such as noribogaine one, two, three, or more times per day. Administration may be via transdermal patch, gum, lozenge, sublingual tablet, intranasal, intrapulmonary, oral administration, or other administration.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “alkyl” refers to monovalent saturated aliphatic hydrocarbyl groups having from 1 to 12 carbon atoms, 1 to 10 carbon atoms, preferably 1 to 6 carbon atoms, and more preferably 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “Cₓ alkyl” refers to an alkyl group having x carbon atoms, wherein x is an integer, for example, C₃ refers to an alkyl group having 3 carbon atoms.

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl—C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR³⁸C(O)alkyl, —NR³⁸C(O)substituted alkyl, —NR³⁸C(O)cycloalkyl, —NR³⁸C(O)substituted cycloalkyl, —NR³⁸C(O)cycloalkenyl, —NR³⁸C(O)substituted cycloalkenyl, —NR³⁸C(O)alkenyl, —NR³⁸C(O)substituted alkenyl, —NR³⁸C(O)alkynyl, —NR³⁸C(O)substituted alkynyl, —NR³⁸C(O)aryl, —NR³⁸C(O)substituted aryl, —NR³⁸C(O)heteroaryl, —NR³⁸C(O)substituted heteroaryl, —NR³⁸C(O)heterocyclic, and —NR³⁸C(O)substituted heterocyclic wherein R³⁸ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³⁹R⁴⁰ where R³⁹ and R⁴⁰ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl,—SO₂-aryl, —SO₂-substituted aryl, —SO₂-heteroaryl, —SO₂-substituted heteroaryl, —SO₂-heterocyclic, and —SO₂-substituted heterocyclic and wherein R³⁹ and R⁴⁰ are optionally joined, together with the nitrogen bound thereto to form a heterocyclic or substituted heterocyclic group, provided that R³⁹ and R⁴⁰ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R³⁹ is hydrogen and R⁴⁰ is alkyl, the substituted amino group is sometimes referred to herein as alkylamino. When R³⁹ and R⁴⁰ are alkyl, the substituted amino group is sometimes referred to herein as dialkylamino. When referring to a monosubstituted amino, it is meant that either R³⁹ or R⁴⁰ is hydrogen but not both. When referring to a disubstituted amino, it is meant that neither R³⁹ nor R⁴⁰ are hydrogen.

“Aminocarbonyl” refers to the group —C(O)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR³⁸ C(O)NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR³⁸C(S)NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR³⁸—SO₂NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁴³)NR⁴¹R⁴² where _{R}⁴¹, R⁴², and R⁴³ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)—which is equivalent to —C(═O)—.

“Carboxy” or “carboxyl” refers to —COOH or salts thereof

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR³⁸—C(O)O-alkyl, —NR³⁸—C(O)O-substituted alkyl, —NR³⁸—C(O)O-alkenyl, —NR³⁸—C(O)O-substituted alkenyl, —NR³⁸—C(O)O-alkynyl, —NR³⁸—C(O)O-substituted alkynyl, —NR³⁸—C(O)O-aryl, —NR³⁸—C(O)O-substituted aryl, —NR³⁸—C(O)O-cycloalkyl, —NR³⁸—C(O)O-substituted cycloalkyl, —NR³⁸—C(O)O-cycloalkenyl, —NR³⁸—C(O)O-substituted cycloalkenyl, —NR³⁸—C(O)O-heteroaryl, —NR³⁸—C(O)O-substituted heteroaryl, —NR³⁸—C(O)O-heterocyclic, and —NR³⁸—C(O)O-substituted heterocyclic wherein R³⁸ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spirobicyclo groups such as spiro[4.5]dec-8-yl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁴⁴C(═NR⁴⁴)N(R⁴⁴)₂ where each R⁴⁴ is independently selected from the group consisting of hydrogen, alkyl, substituted alkyl, aryl, substituted aryl, heteroaryl, substituted heteroaryl, heterocyclic, and substituted heterocyclic and two R⁴⁴ groups attached to a common guanidino nitrogen atom are optionally joined together with the nitrogen bound thereto to form a heterocyclic or substituted heterocyclic group, provided that at least one R⁴⁴ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5 , 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridyl, pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl), and the term “(substituted sulfonyl)aminocarbonyl” refers to —C(O)NH(substituted sulfonyl), wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl,—OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)-, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Compound” or “compounds” as used herein is meant to include the stereoiosmers and tautomers of the indicated formulas.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

As used herein, the term “phosphate ester” refers to any one of the mono-, di- or triphosphate esters of noribogaine, wherein the mono-, di- or triphosphate ester moiety is bonded to the 12-hydroxy group and/or the indole nitrogen of noribogaine.

As used herein, the term “phosphate ester” refers to any one of the mono-, di- or triphosphate esters of noribogaine, wherein the mono-, di- or triphosphate ester moiety is bonded to the 12-hydroxy group and/or the indole nitrogen of noribogaine.

As used herein, the term “monophosphate” refers to the group —P(O)(OH)₂.

As used herein, the term “diphosphate” refers to the group —P(O)(OH)—OP(O)(OH)₂.

As used herein, the term “triphosphate” refers to the group —P(O)(OH)—(OP(O)(OH))₂OH.

As used herein, the term “ester” as it refers to esters of the mono-, di- or triphosphate group means esters of the monophosphate can be represented by the formula —P(O)(OR⁴⁵)₂, where each R⁴⁵ is independently hydrogen, C₁-C₁₂ alkyl, C₃-C₁₀ cycloalkyl, C₆-C₁₄ aryl, heteroaryl of 1 to 10 carbon atoms and 1 to 4 optionally oxidized heteroatoms selected from the group consisting of oxygen, nitrogen, and sulfur and the like, provided that at least one R⁴⁵ is not hydrogen. Likewise, exemplary esters of the di- or triphosphate can be represented by the formulas —P(O)(OR⁴⁵)—OP(O)(OR⁴⁵)₂ and —P(O)(OR⁴⁵)—(OP(O)(OR⁴⁵))₂OR⁴⁵, where R⁴⁵ is as defined above.

As used herein, the term “hydrolyzable group” refers to a group that can be hydrolyzed to release the free hydroxy group under hydrolysis conditions. Examples of hydrolysable group include, but are not limited to those defined for R above. Preferred hydrolysable groups include carboxyl esters, phosphates and phosphate esters. The hydrolysis may be done by chemical reactions conditions such as base hydrolysis or acid hydrolysis or may be done in vivo by biological processes, such as those catalyzed by a phosphate hydrolysis enzyme. Nonlimiting examples of hydrolysable group include groups linked with an ester-based linker (—C(O)O— or —OC(O)—), an amide-based linker (—C(O)NR⁴⁶— or —NR⁴⁶C(O)—), or a phosphate-linker (—P(O)(OR⁴⁶)—O—, —O—P(S)(OR⁴⁶)—O—, —O—P(S)(SR⁴⁶)—O—, —S—P(O)(OR⁴⁶)—O—, —O—P(O)(OR⁴⁶)—S—, —S—P(O)(OR⁴⁶)—S—, —O—P(S)(OR⁴⁶)—S—, —S—P(S)(OR⁴⁶)—O—, —O—P(O)(R⁴⁶)—O—, —O—P(S)(R⁴⁶)—O—, —S—P(O)(R⁴⁶)—O—, —S—P(S)(R⁴⁶)—O—, —S—P(O)(R⁴⁶)—S—, or —O—P(S)(R⁴⁶)—S—) where R⁴⁶ can be hydrogen or alkyl.

Substituted groups of this invention, as set forth above, do not include polymers obtained by an infinite chain of substituted groups. At most, any substituted group can be substituted up to five times.

“Noribogaine” refers to the compound:

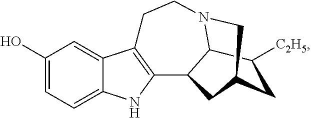

as well as noribogaine derivatives or pharmaceutically acceptable salts and pharmaceutically acceptable solvates thereof. It should be understood that where “noribogaine” is mentioned herein, one more polymorphs of noribogaine can be utilized and are contemplated. In some embodiments, noribogaine is noribogaine glucuronide. Noribogaine can be prepared by demethylation of naturally occurring ibogaine:

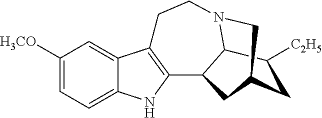

which is isolated from Tabernanth iboga, a shrub of West Africa. Demethylation may be accomplished by conventional techniques such as by reaction with boron tribromide/methylene chloride at room temperature followed by conventional purification. See, for example, Huffman, et al., J. Org. Chem. 50:1460 (1985), which incorporated herein by reference in its entirety. Noribogaine can be synthesized as described, for example in U.S. Patent Pub. Nos. 2013/0165647, 2013/0303756, and 2012/0253037, PCT Patent Publication No. WO 2013/040471 (includes description of making noribogaine polymorphs), and U.S. patent application Ser. No. 13/593,454, each of which is incorporated herein by reference in its entirety.

“Noribogaine derivatives” refer to, without limitation, esters or O-carbamates of noribogaine, or pharmaceutically acceptable salts and/or solvates of each thereof. Also encompassed within this invention are derivatives of noribogaine that act as prodrug forms of noribogaine. A prodrug is a pharmacological substance administered in an inactive (or significantly less active) form. Once administered, the prodrug is metabolized in vivo into an active metabolite. Noribogaine derivatives include, without limitation, those compounds set forth in U.S. Pat. Nos. 6,348,456 and 8,362,007; as well as in U.S. patent application Ser. No. 13/165,626; and US Patent Application Publication Nos. US2013/0131046; US2013/0165647; US2013/0165425; and US2013/0165414; all of which are incorporated herein by reference. Non-limiting examples of noribogaine derivatives encompassed by this invention are given in more detail in the “Compositions” section below.

In some embodiments, the methods of the present disclosure entail the administration of a prodrug of noribogaine that provides the desired maximum serum concentrations and efficacious average noribogaine serum levels. A prodrug of noribogaine refers to a compound that metabolizes, in vivo, to noribogaine. In some embodiments, the prodrug is selected to be readily cleavable either by a cleavable linking arm or by cleavage of the prodrug entity that binds to noribogaine such that noribogaine is generated in vivo. In one preferred embodiment, the prodrug moiety is selected to facilitate binding to the μ and/or κ receptors in the brain either by facilitating passage across the blood brain barrier or by targeting brain receptors other than the λ and/or κ receptors. Examples of prodrugs of noribogaine are provided in U.S. patent application Ser. No. 13/165,626, the entire content of which is incorporated herein by reference.

This invention is not limited to any particular chemical form of noribogaine or noribogaine derivative, and the drug may be given to patients either as a free base, solvate, or as a pharmaceutically acceptable acid addition salt. In the latter case, the hydrochloride salt is generally preferred, but other salts derived from organic or inorganic acids may also be used. Examples of such acids include, without limitation, those described below as “pharmaceutically acceptable salts” and the like.

“Pharmaceutically acceptable composition” refers to a composition that is suitable for administration to a mammal, preferably a human. Such compositions include various excipients, diluents, carriers, and such other inactive agents well known to the skilled artisan.

“Pharmaceutically acceptable salt” refers to pharmaceutically acceptable salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of noribogaine, in the context of modulating opioid analgesic tolerance, refers to an amount of noribogaine that resensitizes the patient to the opioid analgesic therapy.

The therapeutically effective amount of the compound may be higher or lower, depending on the route of administration used. For example, when direct blood administration (e.g., sublingual, pulmonary and intranasal delivery) is used, a lower dose of the compound may be administered. In one aspect, a therapeutically effective amount of noribogaine or derivative is from about 50 ng to less than 100 μg per kg of body weight. Where other routes of administration are used, a higher dose of the compound may be administered. In one embodiment, the therapeutically effective amount of the compound is from greater than about 1 mg to about 8 mg per kg of body weight per day.

A “therapeutic level” of a drug is an amount of noribogaine, noribogaine derivative, or pharmaceutical salt or solvate thereof that is sufficient to modulate tolerance to an opioid analgesic, but not high enough to pose any significant risk to the patient. Therapeutic levels of drugs can be determined by tests that measure the actual concentration of the compound in the blood of the patient. This concentration is referred to as the “serum concentration.” Where the serum concentration of noribogaine is mentioned, it is to be understood that the term “noribogaine” encompasses any form of noribogaine, including derivatives thereof.

The term “dose” refers to a range of noribogaine, noribogaine derivative, or pharmaceutical salt or solvate thereof that provides a therapeutic serum level of noribogaine when given to a patient in need thereof. The dose is recited in a range, for example from 20 mg to 120 mg, and can be expressed either as milligrams or as mg/kg body weight. The attending clinician will select an appropriate dose from the range based on the patient's weight, age, degree of addiction, health, and other relevant factors, all of which are well within the skill of the art.

The term “unit dose” refers to a dose of drug that is given to the patient to provide therapeutic results, independent of the weight of the patient. In such an instance, the unit dose is sold in a standard form (e.g., 20 mg tablet). The unit dose may be administered as a single dose or a series of subdoses. In some embodiments, the unit dose provides a standardized level of drug to the patient, independent of weight of patient. Many medications are sold based on a dose that is therapeutic to all patients based on a therapeutic window. In such cases, it is not necessary to titrate the dosage amount based on the weight of the patient.

A “sub-therapeutic level” of noribogaine or pharmaceutical salt and/or solvate thereof that is less than the therapeutic level described above. For example, the sub-therapeutic level of noribogaine may be e.g., 80%, 70%, 60%, 50%, 40%, 30%, 20%, or 10% less than a therapeutically effective amount (e.g., 120 mg) of noribogaine, or any subvalue or subrange there between. Sub-therapeutic levels of noribogaine may coincide with “maintenance amounts” of noribogaine which are amounts, less than the therapeutically effective amount, that provide some attenuation and/or prevention of post-acute withdrawal syndrome in a patient. The maintenance amount of the compound is expected to be less than the therapeutically effective amount because the level of inhibition does not need to be as high in a patient who is no longer physically addicted to opioid or opioid-like drug.

As defined herein, a “prophylactically effective amount” of a drug is an amount, typically less than the therapeutically effective amount, that provides attenuation and/or prevention of a disease or disorder or symptoms of a disease or disorder in a patient. For example, the prophylactically effective amount of the compound is expected to be less than the therapeutically effective amount because the level of inhibition does not need to be as high in a patient who no longer has a disease or disorder or symptoms of a disease or disorder (e.g., no longer physically addicted to nicotine). For example, a prophylactically effective amount is preferably 90%, 80%, 70%, 60%, 50%, 40%, 30%, 20%, or 10% less than a therapeutically effective amount. However, a prophylactically effective amount may be the same as the therapeutically effective amount, for example when a patient who is physically addicted to nicotine is administered noribogaine to attenuate cravings for a period of time when nicotine use is not feasible. The prophylactically effective amount may vary for different a diseases or disorders or symptoms of different diseases or disorders.

As defined herein, a “maintenance amount” of a drug or an agent is an amount, typically less than the therapeutically effective amount that provides attenuation and/or prevention of syndrome disease or disorder or symptoms of a disease or disorder in a patient. The maintenance amount of the compound is expected to be less than the therapeutically effective amount because the level of inhibition does not need to be as high in a patient who is no longer physically manifests a disease or disorder or symptoms of a disease or disorder. For example, a maintenance amount is preferably 90%, 80%, 70%, 60%, 50%, 40%, 30%, 20%, or 10% less than a therapeutically effective amount, or any subvalue or subrange there between.

“Treatment,” “treating,” and “treat” are defined as acting upon a disease, disorder, or condition with an agent, such as noribogaine to reduce or ameliorate harmful or any other undesired effects of the disease, disorder, or condition and/or its symptoms. “Treatment,” as used herein, covers the treatment of a human patient, and includes: (a) reducing the risk of occurrence of the condition in a patient determined to be predisposed to the condition but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For purposes of this invention, beneficial or desired clinical results include, but are not limited to modulating, reducing, attenuating, relieving or reversing tolerance to an opioid analgesic compound.

“Nociceptive pain” refers to pain that is sensed by nociceptors, which are the nerves that sense and respond to parts of the body suffering from a damage. The nociceptors can signal tissue irritation, impending injury, or actual injury. When activated, they transmit pain signals (via the peripheral nerves as well as the spinal cord) to the brain. Nociceptive pain is typically well localized, constant, and often has an aching or throbbing quality. A subtype of nociceptive pain includes visceral pain and involves the internal organs. Visceral pain tends to be episodic and poorly localized. Nociceptive pain may be time limited; when the tissue damage heals, the pain typically resolves. However, nociceptive pain related to arthritis or cancer may not be time limited. Nociceptive pain tends to respond to treatment with opiate analgesics, such as, for example, buprenorphin, codeine, hydrocodone, oxycodone, morphine, and the like. Examples of nociceptive pain include, without limitation, pains from sprains, bone fractures, burns, bumps, bruises, inflammatory pain from an infection or arthritic disorder, pains from obstructions, cancer pain, and myofascial pain related to abnormal muscle stresses.

“Neuropathic pain” refers to chronic pain, often due to tissue injury. Neuropathic pain is generally caused by injury or damage to nerve fibers. It may include burning or coldness, “pins and needles” sensations, numbness and/or itching. It may be continuous and/or episodic. Neuropathic pain is difficult to treat, but opioids, including, without limitation, methadone, tramadol, tapentadol, oxycodone, methadone, morphine, levorphanol, and the like. Causes of neuropathic pain include, without limitation, alcoholism; amputation; back, leg, and hip problems; chemotherapy; diabetes; facial nerve problems; HIV/AIDS; multiple sclerosis; shingles; spine surgery; trigeminal neuralgia; fibromyalgia; and the like. In some cases, the cause of neuropathic pain may be unclear or unknown.

“Addictive” refers to a compound that, when administered to a mammal over a period of time, creates dependency in the mammal to that compound. The dependence can be physiological and/or psychological. A therapeutic effect of an addictive compound on a mammal may decrease with prolonged administration of the addictive compound, which is a non-limiting example of a physiological dependence. When administered to a mammal, an addictive compound may also create a craving in the mammal for more of it, which is a non-limiting example of a psychological dependence. Examples of addictive compounds include, without limitation, addictive opioids, and the like. In contrast, noribogaine is a non-addictive alkaloid.

“Opioid” refers to a natural product or derivative thereof containing a basic nitrogen atom, typically as part of a cyclic ring structure and less commonly as an acyclic moiety, and synthetic derivatives thereof. Opioids include compounds extracted from poppy pods and their semi-synthetic counterparts which bind to the opiate receptors. Examples of opioids include, without limitation, buprenorphine, codeine, heroine, hydrocodone, oxycodone, morphine, thebaine, and their derivatives, which will be well known to the skilled artisan.

“Analgesic” and “analgesic agent” refer to a compound that is capable of inhibiting and/or reducing pain in mammals. Pain may be inhibited and/or reduced in the mammal by the binding of the opioid analgesic agent to the mu receptor. When analgesia is effected through the mu receptor, the analgesic agent is referred to as a mu receptor agonist. Certain analgesic agents are capable of inhibiting nociceptive and/or neuropathic pain including, by way of example, morphine, codeine, hydromorphone, oxycodone, hydrocodone, buprenorphin, and the like.

As used herein, the term “patient” refers to mammals and includes humans and non-human mammals.

As used herein, the term “QT interval” refers to the measure of the time between the start of the Q wave and the end of the T wave in the electrical cycle of the heart. Prolongation of the QT interval refers to an increase in the QT interval.

The term “tolerance” as used herein refers to the psychological and/or physiologic process wherein the patient adjusts to the frequent presence of a substance such that a higher dose of the substance is required to achieve the same effect. Tolerance may develop at different times for different effects of the same drug (e.g., analgesic effect versus side effects). The mechanisms of tolerance are not entirely understood, but they may include receptor down-regulation or desensitization, inhibitory pathway up-regulation, increased metabolism, and/or changes in receptor processing (e.g., phosphorylation).

A “pharmaceutically acceptable solvate” or “hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

As used herein the term “solvate” is taken to mean that a solid-form of a compound that crystallizes with one or more molecules of solvent trapped inside. A few examples of solvents that can be used to create solvates, such as pharmaceutically acceptable solvates, include, but are certainly not limited to, water, methanol, ethanol, isopropanol, butanol, C1-C6 alcohols in general (and optionally substituted), tetrahydrofuran, acetone, ethylene glycol, propylene glycol, acetic acid, formic acid, water, and solvent mixtures thereof. Other such biocompatible solvents which may aid in making a pharmaceutically acceptable solvate are well known in the art and applicable to the present invention. Additionally, various organic and inorganic acids and bases can be added or even used alone as the solvent to create a desired solvate. Such acids and bases are known in the art. When the solvent is water, the solvate can be referred to as a hydrate. Further, by being left in the atmosphere or recrystallized, the compounds of the present invention may absorb moisture, may include one or more molecules of water in the formed crystal, and thus become a hydrate. Even when such hydrates are formed, they are included in the term “solvate”. Solvate also is meant to include such compositions where another compound or complex co-crystallizes with the compound of interest. The term “solvate” as used herein refers to complexes with solvents in which noribogaine is reacted or from which noribogaine is precipitated or crystallized. For example, a complex with water is known as a “hydrate”. Solvates of noribogaine are within the scope of the invention. It will be appreciated by those skilled in organic chemistry that many organic compounds can exist in more than one crystalline form. For example, crystalline form may vary based on the solvate used. Thus, all crystalline forms of noribogaine or the pharmaceutically acceptable solvates thereof are within the scope of the present invention.

### II. Compositions

As will be apparent to the skilled artisan upon reading this disclosure, this invention provides compositions for reducing tolerance to opioids in a patient undergoing opioid analgesic treatment for pain, comprising noribogaine, noribogaine derivatives, prodrugs of noribogaine, pharmaceutically acceptable salts and/or solvates of each thereof

In some embodiments, the composition is formulated for oral, transdermal, internal, pulmonary, rectal, nasal, vaginal, lingual, intravenous, intraarterial, intramuscular, intraperitoneal, intracutaneous or subcutaneous delivery. In one embodiment, the therapeutically effective amount of the compound is from about 1 mg to about 4 mg per kg body weight per day. In another embodiment, the therapeutically effective amount of the compound is from about 1 mg to about 3 mg per kg body weight per day. In another embodiment, the therapeutically effective amount of the compound is from about 1 mg to about 2 mg per kg body weight per day. In another embodiment, the therapeutically effective amount of the compound is from about 1.3 mg to about 4 mg per kg body weight per day. In another embodiment, the therapeutically effective amount of the compound is from about 1.5 mg to about 3 mg per kg body weight per day. In another embodiment, the therapeutically effective amount of the compound is from about 1.7 mg to about 3 mg per kg body weight per day. In another embodiment, the therapeutically effective amount of the compound is from about 2 mg to about 4 mg per kg body weight per day. In another embodiment, the therapeutically effective amount of the compound is from about 2 mg to about 3 mg per kg body weight per day. The ranges include both extremes as well as any subranges there between.

In one embodiment, the therapeutically effective amount of the compound is from about 1 mg to about 8 mg per kg body weight per day. In another embodiment, the therapeutically effective amount of the compound is from about 1.3 mg to about 7 mg per kg body weight per day. In another embodiment, the therapeutically effective amount of the compound is from about 1.3 mg to about 6 mg per kg body weight per day. In another embodiment, the therapeutically effective amount of the compound is from about 1.3 mg to about 5 mg per kg body weight per day. In another embodiment, the therapeutically effective amount of the compound is from about 1.3 mg to about 4 mg per kg body weight per day. In another embodiment, the therapeutically effective amount of the compound is from about 1.3 mg to about 2 mg per kg body weight per day. In another embodiment, the therapeutically effective amount of the compound is from about 1.5 mg to about 3 mg per kg body weight per day. In another embodiment, the therapeutically effective amount of the compound is from about 1.7 mg to about 3 mg per kg body weight per day. In another embodiment, the therapeutically effective amount of the compound is from about 2 mg to about 4 mg per kg body weight per day. In another embodiment, the therapeutically effective amount of the compound is from about 2 mg to about 3 mg per kg body weight per day.

In one embodiment, the therapeutically effective amount of the compound is about 8 mg/kg body weight per day. In one embodiment, the therapeutically effective amount of the compound is about 7 mg/kg body weight per day. In one embodiment, the therapeutically effective amount of the compound is about 6 mg/kg body weight per day. In one embodiment, the therapeutically effective amount of the compound is about 5 mg/kg body weight per day. In one embodiment, the therapeutically effective amount of the compound is about 4 mg/kg body weight per day. In one embodiment, the therapeutically effective amount of the compound is about 3 mg/kg body weight per day. In one embodiment, the therapeutically effective amount of the compound is about 2 mg/kg body weight per day. In one embodiment, the therapeutically effective amount of the compound is about 1 mg/kg body weight per day.

In one embodiment, the therapeutically effective amount of the compound is about 4 mg/kg body weight per day. In one embodiment, the therapeutically effective amount of the compound is about 3 mg/kg body weight per day. In another embodiment, the therapeutically effective amount of the compound is about 2 mg per kg body weight per day. In another embodiment, the therapeutically effective amount of the compound is about 1.7 mg per kg body weight per day. In another embodiment, the therapeutically effective amount of the compound is about 1.5 mg per kg body weight per day. In another embodiment, the therapeutically effective amount of the compound is about 1.2 mg per kg body weight per day. In another embodiment, the therapeutically effective amount of the compound is about 1 mg per kg body weight per day.

In one aspect, the invention provides a pharmaceutical composition comprising a therapeutically or prophylactically effective amount of noribogaine and a pharmaceutically acceptable excipient, wherein the therapeutically or prophylactically effective amount of noribogaine is an amount that delivers an aggregate amount of noribogaine of about 50 ng to less than 10 μg per kg body weight per day. In some aspects, the therapeutically or prophylactically effective amount of noribogaine is an amount that delivers an aggregate amount of noribogaine of about 50 ng to about 5 μg per kg body weight per day. In some aspects, the therapeutically or prophylactically effective amount of noribogaine is an amount that delivers an aggregate amount of noribogaine of about 50 ng to about 1 μg per kg body weight per day. In some aspects, the composition is formulated for administration once per day. In some aspects, the composition is formulated for administration two or more times per day.

In some embodiments, the composition is formulated for sublingual, intranasal, or intrapulmonary delivery. In one aspect, the invention provides a pharmaceutical composition comprising a pharmaceutically effective amount of noribogaine and a pharmaceutically acceptable excipient, wherein the therapeutically effective amount of noribogaine is an amount that delivers an aggregate amount of noribogaine of 50 ng to less than 100 μg per kg body weight per day. In some aspects, the therapeutically effective amount of noribogaine is an amount that delivers an aggregate amount of noribogaine of 50 ng to 50 μg per kg body weight per day. In some aspects, the therapeutically effective amount of noribogaine is an amount that delivers an aggregate amount of noribogaine of 50 ng to 10 μg per kg body weight per day. In some aspects, the therapeutically effective amount of noribogaine is an amount that delivers an aggregate amount of noribogaine of 50 ng to 1 μg per kg body weight per day. In some aspects, the composition is formulated for administration once per day. In some aspects, the composition is formulated for administration two or more times per day. The ranges include both extremes as well as any subranges there between.

In another embodiment, the therapeutically effective amount of the compound is from 1.3 mg to 4 mg per kg body weight per day. In another embodiment, the therapeutically effective amount of the compound is from 1.5 mg to 3 mg per kg body weight per day. In another embodiment, the therapeutically effective amount of the compound is from 1.7 mg to 3 mg per kg body weight per day. In another embodiment, the therapeutically effective amount of the compound is from 2 mg to 4 mg per kg body weight per day. In another embodiment, the therapeutically effective amount of the compound is from 2 mg to 3 mg per kg body weight per day.

### Compounds Utilized

In one embodiment, the noribogaine derivative is represented by Formula I:

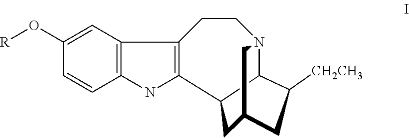

or a pharmaceutically acceptable salt and/or solvate thereof, wherein R is hydrogen or a hydrolyzable group such as hydrolyzable esters of from about 1 to 12 carbons.

Generally, in the above formula, R is hydrogen or a group of the formula:

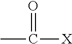

wherein X is a C₁-C₁₂ group, which is unsubstituted or substituted. For example, X may be a linear alkyl group such as methyl, ethyl, n-propyl, n-butyl, n-pentyl, n-hexyl, n-heptyl, n-octyl, n-nonyl, n-decyl, n-undecyl or n-dodecyl, or a branched alkyl group, such as i-propyl or sec-butyl. Also, X may be a phenyl group or benzyl group, either of which may be substituted with lower alkyl groups or lower alkoxy groups. Generally, the lower alkyl and/or alkoxy groups have from 1 to about 6 carbons. For example, the group R may be acetyl, propionyl or benzoyl. However, these groups are only exemplary.

Generally, for all groups X, they may either be unsubstituted or substituted with lower alkyl or lower alkoxy groups. For example, substituted X may be o-, m- or p-methyl or methoxy benzyl groups.

C₁-C₁₂ groups include C₁-C₁₂ alkyl, C₃-C₁₂ cycloalkyl, C₆-C₁₂ aryl, C₇-C₁₂ arylalkyl, wherein Cₓ indicates that the group contains x carbon atoms. Lower alkyl refers to C₁-C₄ alkyl and lower alkoxy refers to C₁-C₄ alkoxy.

In one embodiment, the noribogaine derivative is represented by Formula II:

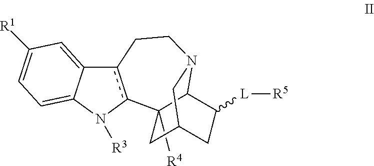

or a pharmaceutically acceptable salt and/or solvate thereof,
wherein
   [symbol] is a single or double bond;
   R¹ is halo, OR², or C₁-C₁₂ alkyl optionally substituted with 1 to 5 R¹⁰;
   R² is hydrogen or a hydrolysable group selected from the group consisting of —C(O)R^{x}, —C(O)OR^{x} and —C(O)N(R^{y})₂ where each R^{x} is selected from the group consisting of C₁-C₆ alkyl optionally substituted with 1 to 5 R¹⁰, and each R⁶ is independently selected from the group consisting of hydrogen, C₁-C₆ alkyl optionally substituted with 1 to 5 R¹⁰, C₆-C₁₄ aryl optionally substituted with 1 to 5 R¹⁰ , C₃-C₁₀ cycloalkyl optionally substituted with 1 to 5 R¹⁰, C₁-C₁₀ heteroaryl having 1 to 4 heteroatoms and which is optionally substituted with 1 to 5 R¹⁰, C₁-C₁₀ heterocyclic having 1 to 4 heteroatoms and which is optionally substituted with 1 to 5 R¹⁰ , and where each R^{y}, together with the nitrogen atom bound thereto form a C₁-C₆ heterocyclic having 1 to 4 heteroatoms and which is optionally substituted with 1 to 5 R¹⁰ or a C₁-C₆ heteroaryl having 1 to 4 heteroatoms and which is optionally substituted with 1 to 5 R¹⁰;
   R³ is selected from the group consisting of hydrogen, C₁-C₁₂ alkyl optionally substituted with 1 to 5 R¹⁰, aryl optionally substituted with 1 to 5 R¹⁰, —C(O)R⁶, —C(O)NR⁶R⁶ and —C(O)OR⁶;
   R⁴ is selected from the group consisting of hydrogen, —(CH₂)ₘOR⁸, —CR⁷(OH)R⁸, —(CH₂)ₘCN, —(CH₂)ₘCOR⁸, —(CH₂)ₘCO₂R⁸, —(CH₂)ₘC(O)NR⁷R⁸, —(C H₂)ₘC(O)NR⁷NR⁸R⁸, —(CH₂)ₘC(O)NR⁷NR⁸C(O)R⁹, and —(CH₂)ₘNR⁷R⁸;
   m is 0, 1, or 2;
   L is a bond or C₁-C₁₂ alkylene;
   R⁵ is selected from the group consisting of hydrogen, C₁-C₁₂ alkyl substituted with 1 to 5 R¹⁰, C₁-C₁₂ alkenyl substituted with 1 to 5 R¹⁰, —X¹—R⁷, —(X¹—Y)ₙ—X¹—R⁷, —SO₂NR⁷R⁸, —O—C(O)R⁹, —C(O)OR⁸, —C(O)NR⁷R⁸, —NR⁷R⁸, —NHC(O)R⁹, and —NR⁷C(O)R⁹;
   each R⁶ is independently selected from the group consisting of hydrogen, C₁-C₁₂ alkyl, C₂-C₁₂ alkenyl, C₂-C₁₂ alkynyl, C₆-C₁₀aryl, C₁-C₆ heteroaryl having 1 to 4 heteroatoms, and C₁-C₆ heterocycle having 1 to 4 heteroatoms, and wherein the alkyl, alkenyl, alkynyl, aryl, heteroaryl, and heterocycle are optionally substituted with 1 to 5 R¹⁰;
   X¹ is selected from the group consisting of O and S;
   Y is C₁-C₄ alkylene or C₆-C₁₀ arylene, or a combination thereof;
   n is 1, 2, or 3;
   R⁷ and R⁸ are each independently selected from the group consisting of hydrogen, C₁-C₁₂ alkyl optionally substituted with 1 to 5 R¹⁰, C₁-C₆ heterocycle having 1 to 4 heteroatoms and which is optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
   R⁹ is selected from the group consisting of C₁-C₁₂ alkyl optionally substituted with 1 to 5 R¹⁰, C₁-C₆ heterocycle having 1 to 4 heteroatoms optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
   R¹⁰ is selected from the group consisting of C₁-C₄ alkyl, phenyl, halo, —OR¹¹, —CN, —COR¹¹, —CO₂R¹¹, —C(O)NHR¹¹, —NR¹¹R¹¹, —C(O)NR¹¹R¹¹, —C(O)NHNHR¹¹, —C(O)NR¹¹NHR¹¹, —C(O)NR¹¹NR¹¹R¹¹, —C(O)NHNR¹¹C(O)R¹¹, —C(O )NHNHC(O)R¹¹, —SO₂NR¹¹R¹¹, —C(O)NR¹¹NR¹¹C(O)R¹¹, and —C(O)NR¹¹NHC(O)R¹¹; and
   R¹¹is independently hydrogen or C₁-C₁₂ alkyl;
   provided that:
   when L is a bond, then R⁵ is not hydrogen;
   when [symbol] is a double bond, R¹ is an ester hydrolyzable group, R³ and R⁴ are both hydrogen, then -L-R⁵ is not ethyl;
   when [symbol] is a double bond, R¹ is —OH, halo or C₁-C₁₂ alkyl optionally substituted with 1 to 5 R¹⁰, then R⁴ is hydrogen; and
   when [symbol] is a double bond, R¹ is OR², R⁴ is hydrogen, -L-R⁵ is ethyl, then R² is not a hydrolyzable group selected from the group consisting of an ester, amide, carbonate and carbamate.

In one embodiment, the noribogaine derivative is represented by Formula III:

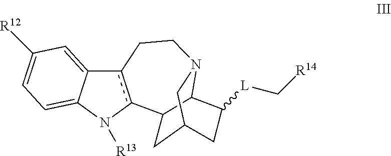

or a pharmaceutically acceptable salt and/or solvate thereof,
wherein
   [symbol] is a single or double bond;
   R¹² is halo, —OH, —SH, —NH₂, —S(O)₂N(R¹⁷)₂, —R^{z}-L¹-R¹⁸, —R^{z}-L¹-R¹⁹, —R^{z}-L¹-R²⁰ or R^{z}-L¹-CHR¹⁸R¹⁹, where R^{z} is O, S or NR¹⁷;
   L¹ is alkylene, arylene, —C(O)-alkylene, —C(O)-arylene, —C(O)O-arylene, —C(O)O-arylene, —C(O)O-alkylene, —C(O)NR²⁰-alkylene, —C(O)NR²⁰-arylene, —C(NR²⁰)NR²⁰-alkylene or —C(NR²⁰)NR²⁰-arylene, wherein L¹ is configured such that —O—L¹—R¹⁸ is —OC(O)-alkylene-R¹⁸, —OC(O)O-arylene-R¹⁸, —OC(O)O-alkylene-R¹⁸, —OC(O)-arylene-R¹⁸, —OC(O)NR²⁰-alkylene-R¹⁸, —OC(O)NR²⁰-arylene-R¹⁸, —OC(NR²⁰)NR²⁰-alkylene-R¹⁸ or —OC(NR²⁰)NR²⁰-arylene-R¹⁸, and wherein the alkylene and arylene are optionally substituted with 1 to 2 R¹⁶;
   R¹³ is hydrogen, —S(O)₂OR²⁰, —S(O)₂R²⁰, —C(O)R¹⁵, —C(O)NR¹⁵R¹⁵, —C(O)OR¹⁵, C₁-C₁₂ alkyl optionally substituted with 1 to 5 R¹⁶, C₁-C₁₂ alkenyl optionally substituted with 1 to 5 R¹⁶, or aryl optionally substituted with 1 to 5 R¹⁶;
   R¹⁴ is hydrogen, halo, —OR¹⁷, —CN, C₁-C₁₂ alkyl, C₁-C₁₂ alkoxy, aryl or aryloxy, where the alkyl, alkoxy, aryl, and aryloxy are optionally substituted with 1 to 5 R¹⁶;
   each R¹⁵ is independently selected from the group consisting of hydrogen, C₁-C₁₂ alkyl, C₂-C₁₂ alkenyl, C₂-C₁₂ alkynyl, aryl, heteroaryl, and heterocycle, and wherein the alkyl, alkenyl, alkynyl, aryl, heteroaryl, and heterocycle are optionally substituted with 1 to 5 R¹⁶;
   R¹⁶ is selected from the group consisting of phenyl, halo, —OR¹⁷, —CN, —COR¹⁷, 13 CO₂R¹⁷, —NR¹⁷R¹⁷, —NR¹⁷C(O)R¹⁷, —NR¹⁷SO₂R¹⁷, —C(O)NR¹⁷R¹⁷, —C (O)NR¹⁷NR¹⁷R¹⁷, —SO₂NR¹⁷R¹⁷ and —C(O)NR¹⁷NR¹⁷C(O)R¹⁷;
   each R^{∫}is independently hydrogen or C₁-C₁₂ alkyl optionally substituted with from 1 to 3 halo;
   R¹⁸ is hydrogen, —C(O)R²⁰, —C(O)OR²⁰, —C(O)N(R²⁰)₂ or —N(R²⁰)C(O)R²⁰;
   R¹⁹ is hydrogen, —N(R²⁰)₂, —C(O)N(R²⁰)₂, —C(NR²⁰)N(R²⁰)₂, —C(NSO₂R²⁰)N(R²⁰)₂, —NR²⁰C(O)N(R²⁰)₂, —NR²⁰C(S)N(R²⁰)₂, —NR²⁰C(NR²⁰)N(R²⁰)₂, —NR²⁰C(NSO₂R²⁰)N(R²⁰ )₂ or tetrazole; and
   each R²⁰ is independently selected from the group consisting of hydrogen, C₁-C₁₂ alkyl and aryl;
   provided that:
   when [symbol] is a double bond and R¹³ and R¹⁴ are hydrogen, then R¹² is not hydroxy;
   when [symbol] is a double bond, R¹⁴ is hydrogen, R¹² is -O-L¹-R¹⁸, —O-L¹-R¹⁹, —O-L¹-R²⁰, and L¹ is alkylene, then —O-L¹-R¹⁸, —O-L¹-R¹⁹, —O—L¹-R²⁰ are not methoxy;
   when [symbol] is a double bond, R¹⁴ is hydrogen, R^{z} is O, L¹ is —C(O)-alkylene, —C(O)-arylene, —C(O)O-arylene, —C(O)O-alkylene, —C(O)NR²⁰-alkylene, or —C(O)NR²⁰-arylene, then none of R¹⁸, R¹⁹ or R²⁰ are hydrogen.

In one embodiment, the noribogaine derivative is represented by Formula IV:

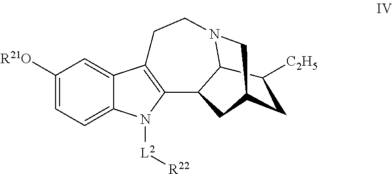

or a pharmaceutically acceptable salt and/or solvate thereof,

wherein

R²¹ is selected from the group consisting of hydrogen, a hydrolysable group selected from the group consisting of —C(O)R²³, —C(O)NR²⁴R²⁵ and —C(O)OR²⁶, where R²³ is selected from the group consisting of hydrogen, alkyl, substituted alkyl, alkenyl, substituted alkenyl, alkynyl and substituted alkynyl, R²⁴ and R²⁵ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic, R²⁶ is selected from the group consisting of alkyl, substituted alkyl, alkenyl, substituted alkenyl, alkynyl, substituted alkynyl, aryl, substituted aryl, heteroaryl, substituted heteroaryl, heterocyclic and substituted heterocyclic, provided that R²¹ is not a saccharide or an oligosaccharide;

L² is selected from the group consisting of a covalent bond and a cleavable linker group;

R²² is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, provided that R is not a saccharide or an oligosaccharide;

provided that when L² is a covalent bond and R²² is hydrogen, then R²¹ is selected from the group consisting of —C(O)NR²⁴R²⁵ and —C(O)OR²⁶; and

further provided that when R²¹ is hydrogen or —C(O)R²³ and L² is a covalent bond, then R²² is not hydrogen.

In one embodiment, the noribogaine derivative is represented by Formula V:

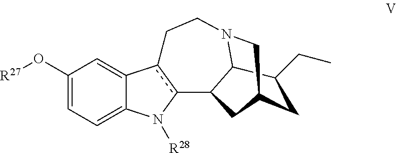

or a pharmaceutically acceptable salt and/or solvate thereof,
wherein:

[symbol] refers to a single or a double bond provided that when [symbol] is a single bond, Formula V refers to the corresponding dihydro compound;

R²⁷ is hydrogen or SO₂OR²⁹;

R²⁸ is hydrogen or SO₂OR²⁹;

R²⁹ is hydrogen or C₁-C₆ alkyl;

provided that at least one of R²⁷ and R²⁸ is not hydrogen.

In one embodiment, the noribogaine derivative is represented by Formula VI:

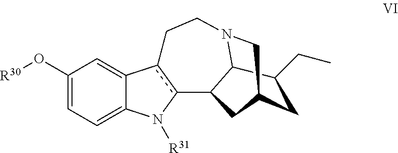

or a pharmaceutically acceptable salt and/or solvate thereof,
wherein:

[symbol] refers to a single or a double bond provided that when [symbol] is a single bond, Formula VI refers to the corresponding vicinal dihydro compound;

R³⁰ is hydrogen, a monophosphate, a diphosphate or a triphosphate; and

R³¹ is hydrogen, a monophosphate, a diphosphate or a triphosphate;

provided that both R³⁰ and R³¹ are not hydrogen; wherein one or more of the monophosphate, diphosphate and triphosphate groups of R³⁰ and R³¹ are optionally esterified with one or more C₁-C₆ alkyl esters.

Noribogaine as utilized herein, can be replaced by a noribogaine derivative or a salt of noribogaine or the noribogaine derivative or a solvate of each of the foregoing.

In a preferred embodiment, the compound utilized herein is noribogaine or a salt thereof. In a more preferred embodiment, the compound utilized herein is noribogaine.

### III. Methods of the Invention

As will be apparent to the skilled artisan upon reading this disclosure, the present invention provides a method for modulating tolerance to opioids in a patient undergoing opioid analgesic therapy, comprising administering to the patient a dosage of noribogaine, noribogaine derivative, or pharmaceutically acceptable salt and/or solvate thereof.

In one aspect of this invention, patient is being treated with an addictive opioid analgesic to relieve the patient's pain. The pain may be of any type and from any source. In one embodiment, the patient is treated for acute pain. In one embodiment, the patient is treated for chronic pain. In one embodiment, the patient is treated for nociceptive pain. In one embodiment, the patient is treated for neuropathic pain. In some embodiments, the pain is caused by surgery, diabetes, trigeminal neuralgia, fibromyalgia, cancer, central pain syndrome, tissue damage, physical injury, and the like. In some embodiments, the source of the pain is unknown or unclear.

In one aspect, this invention relates to a method for modulating tolerance to an opioid analgesic in a patient undergoing opioid analgesic therapy, the method comprising interrupting or administering concurrently with said opioid analgesic an amount of noribogaine, noribogaine derivative, or pharmaceutically acceptable salt and/or solvate thereof that provides an average serum concentration of 50 ng/mL to 180 ng/mL, said concentration being sufficient resensitize the patient to the opioid as an analgesic while maintaining a QT interval of less than about 500 ms during said treatment.

In one aspect, this invention relates to a method for modulating tolerance to an opioid analgesic in a patient undergoing opioid analgesic therapy, the method comprising interrupting or administering concurrently with said opioid analgesic an amount of noribogaine, noribogaine derivative, or pharmaceutically acceptable salt and/or solvate thereof that provides an average serum concentration of 60 ng/mL to 180 ng/mL, said concentration being sufficient to resensitize the patient to the opioid as an analgesic while maintaining a QT interval of less than about 500 ms during said treatment. In some embodiments, the concentration is sufficient to resensitize the patient to the opioid as an analgesic while maintaining a QT interval of less than about 470 ms during treatment. Preferably, the concentration is sufficient to resensitize the patient to the opioid as an analgesic while maintaining a QT interval of less than about 450 ms during treatment. In one embodiment, the concentration is sufficient to resensitize the patient to the opioid as an analgesic while maintaining a QT interval of less than about 420 ms during treatment.

In one embodiment, the QT interval is not prolonged more than about 50 ms. In one embodiment, the QT interval is not prolonged more than about 40 ms. In one embodiment, the QT interval is not prolonged more than about 30 ms. In one embodiment, the QT interval is not prolonged more than about 20 ms. In one embodiment, the QT interval is not prolonged more than about 10 ms.

In one embodiment, the average serum concentration of noribogaine is from 50 ng/mL to 180 ng/mL, or 60 ng/mL to 180 ng/mL. In one embodiment, the average serum concentration of noribogaine is from 50 ng/mL to 150 ng/mL, or 60 ng/mL to 150 ng/mL. In one embodiment, the average serum concentration of noribogaine is from 50 ng/mL to 100 ng/mL, or 60 ng/mL to 100 ng/mL. In one embodiment, the average serum concentration of noribogaine is from 80 ng/mL to 100 ng/mL. The ranges include both extremes as well as any subranges between.

In one embodiment, the dosage or aggregate dosage of noribogaine, noribogaine derivative, or salt or solvate thereof is from about 1 mg/kg to about 4 mg/kg body weight per day. The aggregate dosage is the combined dosage, for example the total amount of noribogaine, noribogaine derivative, or pharmaceutically acceptable salt or solvate thereof administered over a 24-hour period where smaller amounts are administered more than once per day. In one embodiment, the dosage or aggregate dosage of noribogaine, noribogaine derivative, or salt or solvate thereof is from about 1.3 mg/kg to about 4 mg/kg body weight. In one embodiment, the dosage or aggregate dosage of noribogaine, noribogaine derivative, or salt or solvate thereof is from about 1 mg/kg to about 3 mg/kg body weight. In one embodiment, the dosage or aggregate dosage of noribogaine, noribogaine derivative, or salt or solvate thereof is from about 1 mg/kg to about 2 mg/kg body weight. In one embodiment, the dosage or aggregate dosage of noribogaine, noribogaine derivative, or salt or solvate thereof is from about 1.5 mg/kg to about 3 mg/kg body weight. In one embodiment, the dosage or aggregate dosage of noribogaine, noribogaine derivative, or salt or solvate thereof is from about 1.7 mg/kg to about 3 mg/kg body weight. In one embodiment, the dosage or aggregate dosage of noribogaine, noribogaine derivative, or salt or solvate thereof is from about 2 mg/kg to about 4 mg/kg body weight. In one embodiment, the dosage or aggregate dosage of noribogaine, noribogaine derivative, or salt or solvate thereof is from about 2 mg/kg to about 3 mg/kg body weight. In one embodiment, the dosage or aggregate dosage of noribogaine, noribogaine derivative, or salt or solvate thereof is about 2 mg/kg body weight. The ranges include both extremes as well as any subranges there between.

In one embodiment, the dosage or aggregate dosage of noribogaine, noribogaine derivative, or salt or solvate thereof is about 4 mg/kg body weight per day. In one embodiment, the dosage or aggregate dosage of noribogaine, noribogaine derivative, or salt or solvate thereof is about 3 mg/kg body weight per day. In one embodiment, the dosage or aggregate dosage of noribogaine, noribogaine derivative, or salt or solvate thereof is about 2 mg/kg body weight per day. In one embodiment, the dosage or aggregate dosage of noribogaine, noribogaine derivative, or salt or solvate thereof is about 1.7 mg/kg body weight per day. In one embodiment, the dosage or aggregate dosage of noribogaine, noribogaine derivative, or salt or solvate thereof is about 1.6 mg/kg body weight per day. In one embodiment, the dosage or aggregate dosage of noribogaine, noribogaine derivative, or salt or solvate thereof is about 1.5 mg/kg body weight per day. In one embodiment, the dosage or aggregate dosage of noribogaine, noribogaine derivative, or salt or solvate thereof is about 1.4 mg/kg body weight per day. In one embodiment, the dosage or aggregate dosage of noribogaine, noribogaine derivative, or salt or solvate thereof is about 1.3 mg/kg body weight per day. In one embodiment, the dosage or aggregate dosage of noribogaine, noribogaine derivative, or salt or solvate thereof is about 1.2 mg/kg body weight per day. In one embodiment, the dosage or aggregate dosage of noribogaine, noribogaine derivative, or salt or solvate thereof is about 1.1 mg/kg body weight per day. In one embodiment, the dosage or aggregate dosage of noribogaine, noribogaine derivative, or salt and/or solvate thereof is about 1 mg/kg body weight per day.

In one embodiment, the dosage or aggregate dosage of noribogaine, noribogaine derivative, or salt or solvate thereof is between about 10 mg and about 100 mg. In one embodiment, the dosage or aggregate dosage of noribogaine, noribogaine derivative, or salt or solvate thereof is between about 20 mg and about 100 mg. In one embodiment, the dosage or aggregate dosage of noribogaine, noribogaine derivative, or salt or solvate thereof is between about 30 mg and about 100 mg. In one embodiment, the dosage or aggregate dosage of noribogaine, noribogaine derivative, or salt or solvate thereof is between about 40 mg and about 100 mg. In one embodiment, the dosage or aggregate dosage of noribogaine, noribogaine derivative, or salt or solvate thereof is between about 50 mg and about 100 mg. In one embodiment, the dosage or aggregate dosage of noribogaine, noribogaine derivative, or salt or solvate thereof is between about 60 mg and about 100 mg. In one embodiment, the dosage or aggregate dosage of noribogaine, noribogaine derivative, or salt or solvate thereof is between about 60 mg and about 90 mg. In one embodiment, the dosage or aggregate dosage of noribogaine, noribogaine derivative, or salt or solvate thereof is between about 60 mg and about 80 mg. In one embodiment, the dosage or aggregate dosage of noribogaine, noribogaine derivative, or salt or solvate thereof is between about 60 mg and about 70 mg.

In one embodiment, the dosage or aggregate dosage of noribogaine, noribogaine derivative, or salt or solvate thereof is between about 70 mg and about 150 mg. In one embodiment, the dosage or aggregate dosage of noribogaine, noribogaine derivative, or salt or solvate thereof is between about 75 mg and about 150 mg. In one embodiment, the dosage or aggregate dosage of noribogaine, noribogaine derivative, or salt or solvate thereof is between about 80 mg and about 140 mg. In one embodiment, the dosage or aggregate dosage of noribogaine, noribogaine derivative, or salt or solvate thereof is between about 90 mg and about 140 mg. In one embodiment, the dosage or aggregate dosage of noribogaine, noribogaine derivative, or salt or solvate thereof is between about 90 mg and about 130 mg. In one embodiment, the dosage or aggregate dosage of noribogaine, noribogaine derivative, or salt or solvate thereof is between about 100 mg and about 130 mg. In one embodiment, the dosage or aggregate dosage of noribogaine, noribogaine derivative, or salt or solvate thereof is between about 110 mg and about 130 mg.

In another embodiment, there is provided a unit dose of noribogaine, noribogaine derivative, or salt or solvate thereof which is about 120 mg per dose. It being understood that the term “unit dose” means a dose sufficient to provide therapeutic results whether given all at once or serially over a period of time.

In some embodiments, the patient is administered an initial dose of noribogaine, noribogaine derivative, or pharmaceutically acceptable salt or solvate thereof, followed by one or more additional doses. In one embodiment, such a dosing regimen provides an average serum concentration of noribogaine of about 50 ng/mL to about 180 ng/mL. In one embodiment, the one or more additional doses maintain an average serum concentration of about 50 ng/mL to about 180 ng/mL over a period of time.

In some embodiments, the initial dose of noribogaine, noribogaine derivative, or salt or solvate thereof is from about 75 mg to about 120 mg. In one embodiment, the initial dose is about 75 mg. In one embodiment, the initial dose is about 80 mg. In one embodiment, the initial dose is about 85 mg. In one embodiment, the initial dose is about 90 mg. In one embodiment, the initial dose is about 95 mg. In one embodiment, the initial dose is about 100 mg. In one embodiment, the initial dose is about 105 mg. In one embodiment, the initial dose is about 110 mg. In one embodiment, the initial dose is about 115 mg. In one embodiment, the initial dose is about 120 mg.

In some embodiments, the one or more additional doses are lower than the initial dose. In one embodiment, the one or more additional doses are from 5 mg to 50 mg. In one embodiment, the one or more additional doses may or may not comprise the same amount of noribogaine, noribogaine derivative, or salt or solvate thereof. In one embodiment, at least one additional dose is about 5 mg. In one embodiment, at least one additional dose is about 10 mg. In one embodiment, at least one additional dose is about 15 mg. In one embodiment, at least one additional dose is about 20 mg. In one embodiment, at least one additional dose is about 25 mg. In one embodiment, at least one additional dose is about 30 mg. In one embodiment, at least one additional dose is about 35 mg. In one embodiment, at least one additional dose is about 40 mg. In one embodiment, at least one additional dose is about 45 mg. In one embodiment, at least one additional dose is about 50 mg.

In one embodiment, the one or more additional doses are administered periodically. In one embodiment, the one or more additional doses are administered every 4 hours. In one embodiment, the one or more additional doses are administered every 6 hours. In one embodiment, the one or more additional doses are administered every 8 hours. In one embodiment, the one or more additional doses are administered every 10 hours. In one embodiment, the one or more additional doses are administered every 12 hours. In one embodiment, the one or more additional doses are administered every 18 hours. In one embodiment, the one or more additional doses are administered every 24 hours. In one embodiment, the one or more additional doses are administered every 36 hours. In one embodiment, the one or more additional doses are administered every 48 hours.

In some embodiments, the therapeutic dose of noribogaine, noribogaine derivative, or salt or solvate thereof is a tapered dosing over a period of time, during which the patient is detoxified, for example, without suffering significant acute withdrawal symptoms. Without being bound by theory, it is believed that tapering will allow the full therapeutic effect of noribogaine with less prolongation of the QT interval. Tapering involves administration of one or more subsequently lower doses of noribogaine over time. For example, in some embodiments, the first tapered dose is 50% to 95% of the first or original dose. In some embodiments, the second tapered dose is 40% to 90% of the first or original dose. In some embodiments, the third tapered dose is 30% to 85% of the first or original dose. In some embodiments, the fourth tapered dose is 20% to 80% of the first or original dose. In some embodiments, the fifth tapered dose is 10% to 75% of the first or original dose.

In some embodiments, the first tapered dose is given after the first dose of noribogaine. In some embodiments, the first tapered dose is given after the second, third, or a subsequent dose of noribogaine. The first tapered dose may be administered at any time after the previous dose of noribogaine. The first tapered dose can be given once, for example, followed by subsequent further tapered doses, or it can be given multiple times with or without subsequent, further tapered doses (e.g., second, third, fourth, etc. tapered doses), which likewise can be given once or over multiple administrations, for example. In some embodiments, the first tapered dose is administered about one hour, 6 hours, 12 hours, 18 hours, 24 hours, 36 hours, 48 hours, or more after the previous dose of noribogaine. Similarly, second, third, fourth, etc. tapered doses, if given, can be given about one hour, 6 hours, 12 hours, 18 hours, 24 hours, 36 hours, 48 hours, or more after the previous dose of noribogaine.

In some embodiments, one tapered dose is given to achieve the desired lower therapeutic dose. In some embodiments, two tapered doses are given to achieve the desired lower therapeutic dose. In some embodiments, three tapered doses are given to achieve the desired lower therapeutic dose. In some embodiments, four or more tapered doses are given to achieve the desired lower therapeutic dose. Determination of the tapered doses, number of tapered doses, and the like can be readily made a qualified clinician.

In some embodiments, the patient is administered periodically, such as once, twice, three times, four times or five times daily with noribogaine, noribogaine derivative, or a pharmaceutically acceptable salt or solvate thereof. In some embodiments, the administration is once daily, or once every second day, once every third day, three times a week, twice a week, or once a week. The dosage and frequency of the administration depends on the route of administration, dosage, age and body weight of the patient, condition of the patient, opioid analgesic to which tolerance is being modulated, length of time of analgesic treatment, and the like, without limitation. Determination of dosage and frequency suitable for the present technology can be readily made a qualified clinician.

Noribogaine, noribogaine derivative, or a pharmaceutically acceptable salt or solvate thereof, suitable for administration in accordance with the methods provide herein, can be suitable for a variety of delivery modes including, without limitation, oral and transdermal delivery. Compositions suitable for internal, pulmonary, rectal, nasal, vaginal, lingual, intravenous, intra-arterial, intramuscular, intraperitoneal, intracutaneous and subcutaneous routes may also be used. Possible dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences, 16th ed., A. Oslo editor, Easton Pa. 1980).

In a preferred embodiment, noribogaine, noribogaine derivative, or a pharmaceutically acceptable salt and/or solvate thereof is administered orally, which may conveniently be provided in tablet, caplet, sublingual, liquid or capsule form. In certain embodiments, the noribogaine is provided as noribogaine HCl with dosages reported as the amount of free base noribogaine. In some embodiments, the noribogaine HCl is provided in hard gelatin capsules containing only noribogaine HCl with no excipients.

The patient may be receiving any addictive opioid analgesic for the treatment of pain. In a preferred embodiment, the opioid analgesic is selected from the group consisting of fentanyl, hydrocodone, hydromorphone, morphine, oxycodone, buprenorphine, codeine, heroin, thebaine, buprenorphine, methadone, meperidine, tramadol, tapentadol, levorphanol, sufentanil, pentazocine, oxymorphone, and derivatives of each thereof.

### Dosage and Routes of Administration

In some embodiments, the composition is administered via sublingual, intranasal, or intrapulmonary delivery. In one aspect, the invention provides administering a pharmaceutical composition comprising a pharmaceutically effective amount of noribogaine and a pharmaceutically acceptable excipient, wherein the therapeutically effective amount of noribogaine is an amount that delivers an aggregate amount of noribogaine of about 50 ng to about 100 μg per kg body weight per day. In some aspects, the therapeutically effective amount of noribogaine is an amount that delivers an aggregate amount of noribogaine of about 50 ng to about 50 μg per kg body weight per day. In some aspects, the therapeutically effective amount of noribogaine is an amount that delivers an aggregate amount of noribogaine of about 50 ng to about 10 μg per kg body weight per day. In some aspects, the therapeutically effective amount of noribogaine is an amount that delivers an aggregate amount of noribogaine of about 50 ng to about 1 μg per kg body weight per day. In some aspects, the composition is administered once per day. In some aspects, the composition is administered two or more times per day. In some embodiments, the composition is administered less than once a day, for example once every two days, once every three days, once every four days, once a week, etc.

In some embodiments, the composition is administered via oral, buccal, transdermal, internal, pulmonary, rectal, nasal, vaginal, lingual, intravenous, intraarterial, intramuscular, intraperitoneal, intracutaneous or subcutaneous delivery.

In one embodiment, the dosage or aggregate dosage of compound is from about 1 mg to about 4 mg per kg body weight per day. The aggregate dosage is the combined dosage, for example the total amount of noribogaine, noribogaine derivative, or pharmaceutically acceptable salt and/or solvate thereof administered over a 24-hour period where smaller amounts are administered more than once per day.

In some embodiments, the patient is administered periodically, such as once, twice, three times, four times or five times daily with noribogaine, noribogaine derivative, or salt and/or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made by a qualified clinician.

In another embodiment, there is provided a unit dose of noribogaine, noribogaine derivative, or salt or solvate thereof which is about 50 mg to about 200 mg per dose. In one embodiment, the unit dose is about 50 to about 120 mg per dose. In one embodiment, the unit dose is about 120 mg per dose. It being understood that the term “unit dose” means a dose sufficient to provide therapeutic results whether given all at once or serially over a period of time.

In one aspect, this invention relates to a method for attenuating symptoms of anxiety disorder, impulse control disorder, or an anger and/or violence-related disorder in a human patient, comprising administering to the patient a dosage of noribogaine or pharmaceutically acceptable salt and/or solvate thereof that provides an average serum concentration of about 50 ng/mL to about 180 ng/mL, said concentration being sufficient to attenuate said symptoms while maintaining a QT interval of less than about 500 ms during said treatment. In some embodiments, the concentration is sufficient to attenuate said symptoms while maintaining a QT interval of less than about 470 ms during treatment. Preferably, the concentration is sufficient to attenuate said symptoms while maintaining a QT interval of less than about 450 ms during treatment. In one embodiment, the concentration is sufficient to attenuate said symptoms while maintaining a QT interval of less than about 420 ms during treatment.

In one aspect, this invention relates to a method for attenuating food cravings in a human patient, comprising administering to the patient a dosage of noribogaine or pharmaceutically acceptable salt and/or solvate thereof that provides an average serum concentration of about 50 ng/mL to about 400 ng/mL, said concentration being sufficient to attenuate said cravings while maintaining a QT interval of less than about 500 ms during said treatment. In some embodiments, the concentration is sufficient to attenuate said cravings while maintaining a QT interval of less than about 470 ms during treatment. Preferably, the concentration is sufficient to attenuate said cravings while maintaining a QT interval of less than about 450 ms during treatment. In one embodiment, the concentration is sufficient to attenuate said cravings while maintaining a QT interval of less than about 420 ms during treatment.

In one embodiment, the QT interval is not prolonged more than about 50 ms. In one embodiment, the QT interval is not prolonged more than about 40 ms. In one embodiment, the QT interval is not prolonged more than about 30 ms. In a preferred embodiment, the QT interval is not prolonged more than about 20 ms. In one embodiment, the QT interval is not prolonged more than about 10 ms.

Noribogaine, a noribogaine derivative, or a pharmaceutically acceptable salt and/or solvate thereof can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing noribogaine may be prepared using conventional techniques that may include sterile isotonic saline, water, 1,3-butanediol, ethanol, 1,2-propylene glycol, polyglycols mixed with water, Ringer's solution, etc.

### Sustained Treatment

As will be apparent to the skilled artisan upon reading this disclosure, one aspect of the present invention provides a method for treating a condition in a patient, such condition being treatable by noribogaine, noribogaine derivative, or pharmaceutically acceptable salt or solvate thereof, the method comprising administering to the patient an initial unit dose of noribogaine, noribogaine derivative, or pharmaceutically acceptable salt or solvate thereof, followed by at least one additional dose of noribogaine, noribogaine derivative, or pharmaceutically acceptable salt or solvate thereof, such that an average therapeutic serum concentration is achieved by the initial unit dose and maintained by the at least one additional dose.

In one aspect, this invention relates to a method for treating a condition in a patient which is treatable with noribogaine, noribogaine derivative, or pharmaceutically acceptable salt or solvate thereof while maintaining an acceptable QT interval prolongation in said patient, the method comprising:

a) administering to the patient an initial unit dose of noribogaine, noribogaine derivative, or pharmaceutically acceptable salt or solvate thereof, wherein said unit dose provides a therapeutic average serum concentration of 50 ng/mL to 180 ng/mL which serum concentration imparts minimal QT interval prolongation; and

b) maintaining said serum concentration by periodically administering at least one additional dose of noribogaine, noribogaine derivative, or pharmaceutically acceptable salt or solvate thereof, such that the at least one additional dose maintains the average serum concentration of 50 ng/mL to 180 ng/mL during treatment

wherein said additional dose or doses are continued as necessary to treat said condition.

In one aspect, the serum concentration provides a maximum QT interval of less than about 500 ms during said treatment. In some embodiments, the serum concentration provides a maximum QT interval of less than about 470 ms during treatment. Preferably, the serum concentration provides a maximum QT interval of less than about 450 ms during treatment. In one embodiment, the serum concentration provides a maximum QT interval of less than about 420 ms during treatment.

In one embodiment, the QT interval is not prolonged more than about 50 ms. In one embodiment, the QT interval is not prolonged more than about 40 ms. In one embodiment, the QT interval is not prolonged more than about 30 ms. In one embodiment, the QT interval is not prolonged more than about 20 ms. In one embodiment, the QT interval is not prolonged more than about 10 ms.

In one embodiment, the average serum concentration of noribogaine is from 50 ng/mL to 180 ng/mL, or 60 ng/mL to 180 ng/mL. In one embodiment, the average serum concentration of noribogaine is from 50 ng/mL to 150 ng/mL, or 60 ng/mL to 150 ng/mL. In one embodiment, the average serum concentration of noribogaine is from 50 ng/mL to 100 ng/mL, or 60 ng/mL to 100 ng/mL. In one embodiment, the average serum concentration of noribogaine is from 80 ng/mL to 150 ng/mL. In one embodiment, the average serum concentration of noribogaine is from 80 ng/mL to 100 ng/mL. In some aspects of the invention, a lower serum concentration may be therapeutic for a given condition. In one embodiment, the therapeutic serum concentration is between 1 ng/mL and 10 ng/mL. The ranges above include both extremes as well as any subranges between.

In some embodiments, the initial unit dose of noribogaine, noribogaine derivative, or salt or solvate thereof is from 50 mg to 120 mg. In one embodiment, the initial dose is about 50 mg. In one embodiment, the initial dose is about 55 mg. In one embodiment, the initial dose is about 60 mg. In one embodiment, the initial dose is about 65 mg. In one embodiment, the initial dose is about 70 mg. In one embodiment, the initial dose is about 75 mg. In one embodiment, the initial dose is about 80 mg. In one embodiment, the initial dose is about 85 mg. In one embodiment, the initial dose is about 90 mg. In one embodiment, the initial dose is about 95 mg. In one embodiment, the initial dose is about 100 mg. In one embodiment, the initial dose is about 105 mg. In one embodiment, the initial dose is about 110 mg. In one embodiment, the initial dose is about 115 mg. In one embodiment, the initial dose is about 120 mg.

In some embodiments, the initial unit dose of noribogaine, noribogaine derivative, or salt or solvate thereof is administered as subdoses, such that the aggregate dose achieves the unit dose. In some embodiments, the initial unit dose is administered as subunit doses, which subunit doses are administered serially until the unit dose level is achieved, wherein the aggregate of subunit doses provides the initial unit dose and further provides the therapeutic average serum concentration. In some embodiments, the aggregate dose provides a therapeutic serum concentration of 80 ng/mL to 150 ng/mL. In some embodiments, the subdoses are administered every 15 minutes to 6 hours until the unit dose is achieved. In some embodiments, the subdoses are administered every 15 minutes, every 30 minutes, every 1 hour, every 2 hours, every 3 hours, every 4 hours, every 5 hours, or every 6 hours until the unit dose is achieved. The ranges above include both extremes as well as any subranges between.

In some embodiments, the one or more additional doses are lower than the initial dose. In one embodiment, the one or more additional doses are from 5 mg to 75 mg. In one embodiment, the one or more additional doses may or may not comprise the same amount of noribogaine, noribogaine derivative, or salt or solvate thereof. In one embodiment, at least one additional dose is about 5 mg. In one embodiment, at least one additional dose is about 10 mg. In one embodiment, at least one additional dose is about 15 mg. In one embodiment, at least one additional dose is about 20 mg. In one embodiment, at least one additional dose is about 25 mg. In one embodiment, at least one additional dose is about 30 mg. In one embodiment, at least one additional dose is about 35 mg. In one embodiment, at least one additional dose is about 40 mg. In one embodiment, at least one additional dose is about 45 mg. In one embodiment, at least one additional dose is about 50 mg. In one embodiment, at least one additional dose is about 55 mg. In one embodiment, at least one additional dose is about 60 mg. In one embodiment, at least one additional dose is about 65 mg. In one embodiment, at least one additional dose is about 70 mg. In one embodiment, at least one additional dose is about 75 mg. The ranges above include both extremes as well as any subranges between.

In one embodiment, the one or more additional doses are administered periodically. In one embodiment, the one or more additional doses are administered every 4 hours to every 48 hours. In a preferred embodiment, the one or more additional doses are administered every 6 hours to every 24 hours. In one embodiment, the one or more additional doses are administered every 4 hours. In one embodiment, the one or more additional doses are administered every 6 hours. In one embodiment, the one or more additional doses are administered every 8 hours. In one embodiment, the one or more additional doses are administered every 10 hours. In one embodiment, the one or more additional doses are administered every 12 hours. In one embodiment, the one or more additional doses are administered every 18 hours. In one embodiment, the one or more additional doses are administered every 24 hours. In one embodiment, the one or more additional doses are administered every 36 hours. In one embodiment, the one or more additional doses are administered every 48 hours. The ranges above include both extremes as well as any subranges between.

In some embodiments, the therapeutic dose of noribogaine, noribogaine derivative, or salt or solvate thereof is a tapered dosing over a period of time, during which the patient is detoxified, for example, without suffering significant acute withdrawal symptoms. Without being bound by theory, it is believed that tapering will allow the full therapeutic effect of noribogaine with less prolongation of the QT interval. Tapering involves administration of one or more subsequently lower doses of noribogaine over time. For example, in some embodiments, the first tapered dose is 50% to 95% of the initial or at least one additional dose. In some embodiments, the second tapered dose is 40% to 90% of the initial or at least one additional dose. In some embodiments, the third tapered dose is 30% to 85% of the initial or at least one additional dose. In some embodiments, the fourth tapered dose is 20% to 80% of the initial or at least one additional dose. In some embodiments, the fifth tapered dose is 10% to 75% of the initial or at least one additional dose.

In some embodiments, the first tapered dose is given after the first dose of noribogaine. In some embodiments, the first tapered dose is given after the second, third, or a subsequent dose of noribogaine. The first tapered dose may be administered at any time after the previous dose of noribogaine. The first tapered dose can be given once, for example, followed by subsequent further tapered doses, or it can be given multiple times with or without subsequent, further tapered doses (e.g., second, third, fourth, etc. tapered doses), which likewise can be given once or over multiple administrations, for example. In some embodiments, the first tapered dose is administered one hour, 6 hours, 12 hours, 18 hours, 24 hours, 36 hours, 48 hours, or more after the previous dose of noribogaine. Similarly, second, third, fourth, etc. tapered doses, if given, can be given one hour, 6 hours, 12 hours, 18 hours, 24 hours, 36 hours, 48 hours, or more after the previous dose of noribogaine.

In some embodiments, the dose is tapered starting 12 to 96 hours after the initial dose. In some embodiments, the dose is tapered starting 12 hours after the initial dose. In some embodiments, the dose is tapered starting 18 hours after the initial dose. In some embodiments, the dose is tapered starting 24 hours after the initial dose. In some embodiments, the dose is tapered starting 30 hours after the initial dose. In some embodiments, the dose is tapered starting 36 hours after the initial dose. In some embodiments, the dose is tapered starting 42 hours after the initial dose. In some embodiments, the dose is tapered starting 48 hours after the initial dose. In some embodiments, the dose is tapered starting 54 hours after the initial dose. In some embodiments, the dose is tapered starting 60 hours after the initial dose. In some embodiments, the dose is tapered starting 66 hours after the initial dose. In some embodiments, the dose is tapered starting 72 hours after the initial dose. In some embodiments, the dose is tapered starting 78 hours after the initial dose. In some embodiments, the dose is tapered starting 84 hours after the initial dose. In some embodiments, the dose is tapered starting 90 hours after the initial dose. In some embodiments, the dose is tapered starting 96 hours after the initial dose.

In some embodiments, at least one additional dose is administered 4 hours to 24 hours after the initial unit dose. In some embodiments, the additional doses are administered 4 hours to 24 hours after the previous dose. In some embodiments, the doses are administered every 4 hours to 24 hours. In some embodiments, the doses are administered as needed. The dosage and frequency of the administration depends on the route of administration, dosage, age and body weight of the patient, condition of the patient, without limitation. Determination of dosage and frequency suitable for the present technology can be readily made a qualified clinician.

In some embodiments, the doses are administered at varying time points. That is, each dose need not be administered at the same interval as the previous dose. In some embodiments, the additional doses are administered more frequently at the beginning of treatment, and less frequently after a certain period of time. For example and without limitation, withdrawal symptoms are the most severe in the first 72 hours after the last dose of the drug of addiction. Noribogaine, noribogaine derivative, or a pharmaceutically acceptable solvate or salt thereof may be administered, for example, every 4 hours to 12 hours for the first 72 hours, and less frequently (e.g., 12 hours to 24 hours) thereafter.

In some embodiments, the noribogaine, noribogaine derivative, or a pharmaceutically acceptable salt or solvate thereof is administered for an indefinite period of time (e.g., for several months or several years, up to the lifetime of the patient).

In some embodiments, the patient undergoes long-term (e.g., one year or longer) treatment with maintenance doses of noribogaine, noribogaine derivative, or salt or solvate thereof. In some embodiments, the patient is first treated for acute symptoms of the condition with therapeutic doses of noribogaine as described above, and then the amount of noribogaine is reduced to maintenance levels, for example after acute symptoms would be expected to have subsided. This is particularly relevant to treating drug addiction, as acute withdrawal symptoms generally are the most pronounced in the first 48 to 72 hours after cessation of the drug of addiction, although acute withdrawal may last as long as a week or more.

### Patient Pre-Screening and Monitoring

Pre-screening of patients before treatment with noribogaine, noribogaine derivative, or pharmaceutically acceptable salt or solvate thereof and/or monitoring of patients during noribogaine treatment may be required to ensure that QT interval is not prolonged beyond a certain value. For example, QT interval greater than about 500 ms can be considered dangerous for individual patients. Pre-screening and/or monitoring may be necessary at high levels of noribogaine treatment.

In one embodiment, a patient receiving a therapeutic dose of noribogaine, noribogaine derivative, or pharmaceutically acceptable salt or solvate thereof is monitored in a clinical setting. Monitoring may be necessary to ensure the QT interval is not prolonged to an unacceptable degree. A “clinical setting” refers to an inpatient setting (e.g., inpatient clinic, hospital, rehabilitation facility) or an outpatient setting with frequent, regular monitoring (e.g., outpatient clinic that is visited daily to receive dose and monitoring). Monitoring includes monitoring of QT interval. Methods for monitoring of QT interval are well-known in the art, for example by ECG.

In one embodiment, a patient receiving a maintenance dose of noribogaine is not monitored in a clinical setting. In one embodiment, a patient receiving a maintenance dose of noribogaine is monitored periodically, for example daily, weekly, monthly, or occasionally.

In one aspect, this invention relates to a method for modulating tolerance to an opioid analgesic in a patient undergoing opioid analgesic therapy, comprising selecting a patient who is prescreened to evaluate the patient's expected tolerance for prolongation of QT interval, administering to the patient a dosage of noribogaine, noribogaine derivative, or pharmaceutically acceptable salt or solvate thereof that provides an average serum concentration of about 50 ng/mL to about 180 ng/mL, said concentration being sufficient to resensitize the patient to the opioid as an analgesic while maintaining a QT interval of less than 500 ms during said treatment. In some embodiments, the concentration is sufficient to resensitize the patient to the opioid as an analgesic while maintaining a QT interval of less than about 470 ms during treatment. Preferably, the concentration is sufficient to resensitize the patient to the opioid as an analgesic while maintaining a QT interval of less than about 450 ms during treatment. In one embodiment, the concentration is sufficient to resensitize the patient to the opioid as an analgesic while maintaining a QT interval of less than about 420 ms during treatment.

In one embodiment, prescreening of the patient comprises ascertaining that noribogaine treatment will not result in a maximum QT interval over about 500 ms. In one embodiment, prescreening of the patient comprises ascertaining that noribogaine treatment will not result in a maximum QT interval over about 470 ms. In one embodiment, prescreening comprises ascertaining that noribogaine treatment will not result in a maximum QT interval over about 450 ms. In one embodiment, prescreening comprises ascertaining that noribogaine treatment will not result in a maximum QT interval over about 420 ms. In one embodiment, prescreening comprises determining the patient's pre-treatment QT interval.

As it relates to pre-screening or pre-selection of patients, patients may be selected based on any criteria as determined by the skilled clinician. Such criteria may include, by way of non-limiting example, pre-treatment QT interval, pre-existing cardiac conditions, risk of cardiac conditions, age, sex, general health, and the like. The following are examples of selection criteria for disallowing noribogaine treatment or restricting dose of noribogaine administered to the patient: high QT interval before treatment (e.g., such that there is a risk of the patient's QT interval exceeding about 500 ms during treatment); congenital long QT syndrome; bradycardia; hypokalemia or hypomagnesemia; recent acute myocardial infarction; uncompensated heart failure; and taking other drugs that increase QT interval. In some embodiments, the methods can include selecting and/or administering/providing noribogaine to a patient that lacks one more of such criteria.

In one embodiment, this invention relates to pre-screening a patient to determine if the patient is at risk for prolongation of the QT interval beyond a safe level. In one embodiment, a patient at risk for prolongation of the QT interval beyond a safe level is not administered noribogaine. In one embodiment, a patient at risk for prolongation of the QT interval beyond a safe level is administered noribogaine at a limited dosage.

In one embodiment, this invention relates to monitoring a patient who is administered a therapeutic dose of noribogaine. In one embodiment, the dose of noribogaine is reduced if the patient has serious adverse side effects. In one embodiment, the noribogaine treatment is discontinued if the patient has serious adverse side effects. In one embodiment, the adverse side effect is a QT interval that is prolonged beyond a safe level. The determination of a safe level of prolongation is within the skill of a qualified clinician.

In one aspect, this invention relates to a method for treating an anxiety disorder, an impulse control disorder, or an anger/violence-related disorder, and/or treating or attenuating the symptoms thereof in a patient, comprising selecting a patient exhibiting symptoms of an anxiety disorder, impulse control disorder, or anger/violence-related disorder who is prescreened to evaluate the patient's expected tolerance for prolongation of QT interval, administering to the patient a dosage of noribogaine, noribogaine derivative, or pharmaceutically acceptable salt or solvate thereof that provides an average serum concentration of about 50 ng/mL to about 850 ng/mL, said concentration being sufficient to inhibit or ameliorate said disorder or symptoms while maintaining a QT interval of less than about 500 ms during said treatment. In some embodiments, the concentration is sufficient to attenuate said symptoms while maintaining a QT interval of less than about 470 ms during treatment. Preferably, the concentration is sufficient to attenuate said symptoms while maintaining a QT interval of less than about 450 ms during treatment. In one embodiment, the concentration is sufficient to attenuate said symptoms while maintaining a QT interval of less than about 420 ms during treatment.

In one aspect, this invention relates to a method for regulating food intake, and/or treating or attenuating food cravings, in a patient, comprising selecting an overweight or obese patient who is prescreened to evaluate the patient's expected tolerance for prolongation of QT interval, administering to the patient a dosage of noribogaine, noribogaine derivative, or pharmaceutically acceptable salt or solvate thereof that provides an average serum concentration of about 50 ng/mL to about 180 ng/mL, said concentration being sufficient to inhibit or ameliorate said disorder or symptoms while maintaining a QT interval of less than about 500 ms during said treatment.

### Kit of Parts

One aspect of this invention is directed to a kit of parts for the modulation of tolerance to an opioid analgesic, wherein the kit comprises a composition comprising noribogaine, noribogaine derivative, or salt or solvate thereof and a means for administering the composition to a patient in need thereof. The means for administration to a patient can include, for example, any one or combination of noribogaine, or a noribogaine derivative, or a pharmaceutically acceptable salt or solvate thereof (e.g., a pill, transdermal patch, injectable, and the like, without limitation) and optionally a means for dispensing and/or administering the formulation (e.g., a syringe, a needle, an IV bag comprising the composition, a vial comprising the composition, an inhaler comprising the composition, etc, without limitation). In one embodiment, the kit of parts further comprises instructions for dosing and/or administration of the composition.

In some aspects, the invention is directed to a kit of parts for administration of noribogaine, the kit comprising multiple delivery vehicles, wherein each delivery vehicle contains a discrete amount of noribogaine and further wherein each delivery vehicle is identified by the amount of noribogaine provided therein; and optionally further comprising a dosing treatment schedule in a readable medium. In some embodiments, the dosing treatment schedule includes the amount of noribogaine required to achieve each average serum level is provided. In some embodiments, the kit of parts includes a dosing treatment schedule that provides an attending clinician the ability to select a dosing regimen of noribogaine based on the sex of the patient, mass of the patient, and the serum level that the clinician desires to achieve. In some embodiments, the dosing treatment schedule further provides information corresponding to the volume of blood in a patient based upon weight (or mass) and sex of the patient. In an embodiment, the storage medium can include an accompanying pamphlet or similar written information that accompanies the unit dose form in the kit. In an embodiment, the storage medium can include electronic, optical, or other data storage, such as a non-volatile memory, for example, to store a digitally-encoded machine-readable representation of such information.

The term “delivery vehicle” as used herein refers to any formulation that can be used for administration of noribogaine to a patient. Non-limiting, exemplary delivery vehicles include caplets, pills, capsules, tablets, powder, liquid, or any other form by which the drug can be administered. Delivery vehicles may be intended for administration by oral, inhaled, injected, or any other means.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

In some aspects, the machine-readable medium comprises software that contains information regarding dosing schedules for the unit dose form of noribogaine, and optionally other drug information. In some embodiments, the software may be interactive, such that the attending clinician or other medical professional can enter patient information. In a non-limiting example, the medical professional may enter the weight and sex of the patient to be treated, and the software program provides a recommended dosing regimen based on the information entered. The amount and timing of noribogaine recommended to be delivered will be within the dosages that result in the serum concentrations as provided herein.

In some embodiments, the kit of parts comprises multiple delivery vehicles in a variety of dosing options. For example, the kit of parts may comprise pills or tablets in multiple dosages, such as 120 mg, 90 mg, 60 mg, 30 mg, 20 mg, 10 mg, and/or 5 mg of noribogaine per pill. Each pill is labeled such that the medical professional and/or patient can easily distinguish different dosages. Labeling may be based on printing or embossing on the pill, shape of the pill, color of pill, the location of the pill in a separate, labeled compartment within the kit, and/or any other distinguishing features of the pill. In some embodiments, all of the delivery vehicles within a kit are intended for one patient. In some embodiments, the delivery vehicles within a kit are intended for multiple patients.

One aspect of this invention is directed to a kit of parts for the modulation of tolerance to an opioid analgesic in a patient undergoing opioid analgesic therapy, wherein the kit comprises a unit dose form of noribogaine, noribogaine derivative, or salt or solvate thereof. The unit dose form provides a patient with an average serum level of noribogaine of from about 50 ng/mL to about 180 ng/mL or about 60 ng/mL to about 180 ng/mL. The unit dose form provides a patient with an average serum level of noribogaine of from about 50 ng/mL to about 800 ng/mL or about 60 ng/mL to about 800 ng/mL. In one embodiment, the unit dose form provides a patient with an average serum level of noribogaine of from about 50 ng/mL to about 400 ng/mL or about 60 ng/mL to about 400 ng/mL. In one embodiment, the unit dose form provides a patient with an average serum level of noribogaine of from 80 ng/mL to 100 ng/mL.

In some embodiments, the unit dose of noribogaine, noribogaine derivative, or pharmaceutically acceptable salt or solvate thereof is from 20 mg to 120 mg. In one embodiment, the unit dose is 20 mg. In one embodiment, the unit dose is 30 mg. In one embodiment, the unit dose is 40 mg. In one embodiment, the unit dose is 50 mg. In one embodiment, the unit dose is 60 mg. In one embodiment, the unit dose is 70 mg. In one embodiment, the unit dose is 80 mg. In one embodiment, the unit dose is 90 mg. In one embodiment, the unit dose is 100 mg. In one embodiment, the unit dose is 110 mg. In one embodiment, the unit dose is 120 mg.

In some embodiments, the unit dose form comprises one or multiple dosages to be administered periodically, such as once, twice, three times, four times or five times daily with noribogaine or its prodrug. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the opioid analgesic employed. Determination of the unit dose form providing a dosage and frequency suitable for a given patient can readily be made by a qualified clinician.

In some embodiments, the initial unit dose and one or more additional doses of noribogaine, noribogaine derivative, or salt or solvate thereof are provided as one or multiple dosages to be administered periodically, such as once, twice, three times, four times or five times daily with noribogaine or its prodrug. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the severity of the condition. Determination of the unit dose form providing a dosage and frequency suitable for a given patient can readily be made by a qualified clinician.

In one aspect, provided herein is a kit of parts comprising two or more doses of noribogaine, noribogaine derivative, or pharmaceutically acceptable salt or solvate thereof, wherein the two or more doses comprise an amount of noribogaine, noribogaine derivative, or pharmaceutically acceptable salt or solvate thereof that is sufficient to maintain a serum concentration of 50 ng/mL to 180 ng/mL when administered to a patient.

In one embodiment, one dose comprises an initial dose of noribogaine, noribogaine derivative, or pharmaceutically acceptable salt or solvate thereof, said initial dose being sufficient to achieve a therapeutic serum concentration when administered to a patient; and at least one additional dose, said additional dose sufficient to maintain a therapeutic serum concentration when administered to a patient, wherein the therapeutic serum concentration is between 50 ng/mL and 180 ng/mL. In another embodiment, the initial dose is from 75 mg to 120 mg. In another embodiment, the at least one additional dose is from 5 mg to 25 mg.

These dose ranges may be achieved by transdermal, oral, or parenteral administration of noribogaine, noribogaine derivative, or a pharmaceutically acceptable salt or solvate thereof in unit dose form. Such unit dose form may conveniently be provided in transdermal patch, tablet, caplet, liquid or capsule form. In certain embodiments, the noribogaine is provided as noribogaine HCl with dosages reported as the amount of free base noribogaine. In some embodiments, the noribogaine HCl is provided in hard gelatin capsules containing only noribogaine HCl with no excipients. In some embodiments, noribogaine is provided in saline for intravenous administration.

### Formulations

This invention further relates to pharmaceutically acceptable formulations comprising a unit dose of noribogaine, noribogaine derivative, or pharmaceutically acceptable salt or solvate thereof, wherein the amount of noribogaine is sufficient to provide an average serum concentration of about 50 ng/mL to about 180 ng/mL when administered to a patient. In a preferred embodiment, the amount of noribogaine is sufficient to provide an average serum concentration of about 80 ng/mL to about 100 ng/mL when administered to a patient. In one embodiment, the amount of noribogaine, noribogaine derivative, or pharmaceutically acceptable salt thereof is an amount that delivers an aggregate amount of noribogaine of about 50 ng to about 10 μg per kg body weight per day.

This invention further relates to pharmaceutically acceptable formulations comprising a unit dose of noribogaine, noribogaine derivative, or pharmaceutically acceptable salt or solvate thereof, wherein the amount of noribogaine is sufficient to provide and/or maintain an average serum concentration of about 50 ng/mL to about 180 ng/mL when administered to a patient. In a preferred embodiment, the amount of noribogaine is sufficient to provide and/or maintain an average serum concentration of 80 ng/mL to 100 ng/mL, when administered to a patient.

In some embodiments, the unit dose of noribogaine is administered in one or more dosings.

In one embodiment, the amount of noribogaine is sufficient to provide an average serum concentration of noribogaine from 50 ng/mL to 180 ng/mL, or 60 ng/mL to 180 ng/mL. In one embodiment, the amount of noribogaine is sufficient to provide an average serum concentration of noribogaine from 50 ng/mL to 150 ng/mL, or 60 ng/mL to 150 ng/mL. In one embodiment, the amount of noribogaine is sufficient to provide an average serum concentration of noribogaine from about 50 ng/mL to about 120 ng/mL, or about 60 ng/mL to about 120 ng/mL. In one embodiment, the amount of noribogaine is sufficient to provide an average serum concentration of noribogaine from about 50 ng/mL to about 100 ng/mL, or about 60 ng/mL to about 100 ng/mL. In one embodiment, the amount of noribogaine is sufficient to provide an average serum concentration of noribogaine from about 50 ng/mL to about 120 ng/mL, or about 60 ng/mL to about 120 ng/mL. In one embodiment, the amount of noribogaine is sufficient to provide an average serum concentration of noribogaine from about 50 ng/mL to about 100 ng/mL, or about 60 ng/mL to about 100 ng/mL. In one embodiment, the amount of noribogaine is sufficient to provide an average serum concentration of noribogaine from about 80 ng/mL to about 100 ng/mL. The ranges include both extremes as well as any subranges between.

In some embodiments, the initial unit dose of noribogaine, noribogaine derivative, or pharmaceutically acceptable salt or solvate thereof is from about 50 mg to about 120 mg. In one embodiment, the unit dose is about 50 mg. In one embodiment, the unit dose is about 55 mg. In one embodiment, the unit dose is 60 mg. In one embodiment, the unit dose is about 65 mg. In one embodiment, the unit dose is about 70 mg. In one embodiment, the unit dose is about 75 mg. In one embodiment, the unit dose is about 80 mg. In one embodiment, the unit dose is about 85 mg. In one embodiment, the unit dose is about 90 mg. In one embodiment, the unit dose is about 95 mg. In one embodiment, the unit dose is about 100 mg. In one embodiment, the unit dose is 105 mg. In one embodiment, the unit dose is about 110 mg. In one embodiment, the unit dose is about 115 mg. In one embodiment, the unit dose is about 120 mg.

In some embodiments, the at least one additional dose of noribogaine, noribogaine derivative, or pharmaceutically acceptable salt or solvate thereof is from 5 mg to 75 mg. In one embodiment, the unit dose is 5 mg. In one embodiment, the unit dose is 10 mg. In one embodiment, the unit dose is 15 mg. In one embodiment, the unit dose is 20 mg. In one embodiment, the unit dose is 25 mg. In one embodiment, the unit dose is 30 mg. In one embodiment, the unit dose is 35 mg. In one embodiment, the unit dose is 40 mg. In one embodiment, the unit dose is 45 mg. In one embodiment, the unit dose is 50 mg. In one embodiment, the unit dose is 55 mg. In one embodiment, the unit dose is 60 mg. In one embodiment, the unit dose is 65 mg. In one embodiment, the unit dose is 70 mg. In one embodiment, the unit dose is 75 mg.

In some embodiments, the formulation comprises a delivery vehicle, as described above. In one embodiment, the delivery vehicle comprises 5 mg to 120 mg noribogaine, noribogaine derivative, or pharmaceutically acceptable salt or solvate thereof

In some embodiments, the formulation is a controlled release formulation. The term “controlled release formulation” includes sustained release and time-release formulations. Controlled release formulations are well-known in the art. These include excipients that allow for sustained, periodic, pulse, or delayed release of the drug. Controlled release formulations include, without limitation, embedding of the drug into a matrix; enteric coatings; micro-encapsulation; gels and hydrogels; implants; transdermal patches; and any other formulation that allows for controlled release of a drug.

In one embodiment, the amount of noribogaine is sufficient to provide an average serum concentration of noribogaine from about 50 ng/mL to about 180 ng/mL, or about 60 ng/mL to about 180 ng/mL. In one embodiment, the amount of noribogaine is sufficient to provide an average serum concentration of noribogaine from about 50 ng/mL to about 150 ng/mL, or about 60 ng/mL to about 150 ng/mL. In one embodiment, the amount of noribogaine is sufficient to provide an average serum concentration of noribogaine from about 50 ng/mL to about 120 ng/mL, or about 60 ng/mL to about 120 ng/mL. In one embodiment, the amount of noribogaine is sufficient to provide an average serum concentration of noribogaine from about 50 ng/mL to about 100 ng/mL, or about 60 ng/mL to about 100 ng/mL. In one embodiment, the amount of noribogaine is sufficient to provide an average serum concentration of noribogaine from about 80 ng/mL to about 100 ng/mL. The ranges include both extremes as well as any subranges between.

In some embodiments, the unit dose of noribogaine, noribogaine derivative, or pharmaceutically acceptable salt or solvate thereof is from about 20 mg to about 120 mg. In one embodiment, the unit dose is about 20 mg. In one embodiment, the unit dose is about 30 mg. In one embodiment, the unit dose is about 40 mg. In one embodiment, the unit dose is about 50 mg. In one embodiment, the unit dose is about 60 mg. In one embodiment, the unit dose is about 70 mg. In one embodiment, the unit dose is about 80 mg. In one embodiment, the unit dose is about 90 mg. In one embodiment, the unit dose is about 100 mg. In one embodiment, the unit dose is about 110 mg. In one embodiment, the unit dose is about 120 mg.

This invention further relates to pharmaceutically acceptable formulations comprising a unit dose of noribogaine, noribogaine derivative, or pharmaceutically acceptable salt or solvate thereof, wherein the amount of noribogaine is sufficient to provide an average serum concentration of about 50 ng/mL to about 850 ng/mL when administered to a patient. In a preferred embodiment, the amount of noribogaine is sufficient to provide an average serum concentration of about 50 ng/mL to about 400 ng/mL when administered to a patient.

In one embodiment, the amount of noribogaine is sufficient to provide an average serum concentration of noribogaine from about 50 ng/mL to about 800 ng/mL or about 60 ng/mL to about 800 ng/mL. In one embodiment, the amount of noribogaine is sufficient to provide an average serum concentration of noribogaine from about 50 ng/mL to about 700 ng/mL or about 60 ng/mL to about 700 ng/mL. In one embodiment, the amount of noribogaine is sufficient to provide an average serum concentration of noribogaine from about 50 ng/mL to about 600 ng/mL, or about 60 ng/mL to about 600 ng/mL. In a preferred embodiment, the amount of noribogaine is sufficient to provide an average serum concentration of noribogaine from about 50 ng/mL to about 500 ng/mL, or about 60 ng/mL to about 500 ng/mL. In one embodiment, the amount of noribogaine is sufficient to provide an average serum concentration of noribogaine from about 50 ng/mL to about 400 ng/mL, or about 60 ng/mL to about 400 ng/mL. In one embodiment, the amount of noribogaine is sufficient to provide an average serum concentration of noribogaine from about 50 ng/mL to about 300 ng/mL, or about 60 ng/mL to about 300 ng/mL. In one embodiment, the amount of noribogaine is sufficient to provide an average serum concentration of noribogaine from about 50 ng/mL to about 200 ng/mL, or about 60 ng/mL to about 200 ng/mL. In one embodiment, the amount of noribogaine is sufficient to provide an average serum concentration of noribogaine from about 50 ng/mL to about 100 ng/mL, or about 60 ng/mL to about 100 ng/mL. The ranges include both extremes as well as any subranges between.

In some embodiments, the formulation is designed for periodic administration, such as once, twice, three times, four times or five times daily with noribogaine, noribogaine derivative, or a pharmaceutically acceptable salt or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made a qualified clinician.

In some embodiments, the formulation designed for administration in accordance with the methods provide herein can be suitable for a variety of delivery modes including, without limitation, oral, transdermal, sublingual, buccal, intrapulmonary or intranasal delivery. Formulations suitable for internal, pulmonary, rectal, nasal, vaginal, lingual, intravenous, intra-arterial, intramuscular, intraperitoneal, intracutaneous and subcutaneous routes may also be used. Possible formulations include tablets, capsules, pills, powders, aerosols, suppositories, parenterals, and oral liquids, including suspensions, solutions and emulsions. Sustained release dosage forms may also be used. All formulations may be prepared using methods that are standard in the art (see e.g., Remington's Pharmaceutical Sciences, 16th ed., A. Oslo editor, Easton Pa. 1980).

In a preferred embodiment, the formulation is designed for oral administration, which may conveniently be provided in tablet, caplet, sublingual, liquid or capsule form. In certain embodiments, the noribogaine is provided as noribogaine HCl with dosages reported as the amount of free base noribogaine. In some embodiments, the noribogaine HCl is provided in hard gelatin capsules containing only noribogaine HCl with no excipients.

Noribogaine or a noribogaine derivative can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing noribogaine may be prepared using conventional techniques that may include sterile isotonic saline, water, 1,3-butanediol, ethanol, 1,2-propylene glycol, polyglycols mixed with water, Ringer's solution, etc.

The compositions utilized herein may be formulated for aerosol administration, particularly to the respiratory tract and including intrapulmonary or intranasal administration. The compound will generally have a small particle size, for example of the order of 5 microns or less. Such a particle size may be obtained by means known in the art, for example by micronization. The active ingredient may be provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively, the active ingredients may be provided in the form of a dry powder, for example a powder mix of the compound in a suitable powder base such as lactose, starch, starch derivatives such as hydroxypropylmethyl cellulose and polyvinylpyrrolidine. In some embodiments, the powder carrier will form a gel in the nasal cavity. The powder composition may be presented in unit dose form, for example in capsules or cartridges, gelatin or blister packs, from which the powder may be administered by means of an inhaler.

The compositions utilized herein may be formulated for sublingual administration, for example as sublingual tablets. Sublingual tablets are designed to dissolve very rapidly. The formulations of these tablets contain, in addition to the drug, a limited number of soluble excipients, usually lactose and powdered sucrose, but sometimes dextrose and mannitol.

It has been discovered that noribogaine has a bitter taste to at least some patients. Accordingly, compositions for oral use (including sublingual, inhaled, and other oral formulations) may be formulated to utilize taste-masking technologies. A number of ways to mask the taste of bitter drugs are known in the art, including addition of sugars, flavors, sweeteners, or coatings; use of lipoproteins, vesicles, and/or liposomes; granulation; microencapsulation; numbing of taste buds; multiple emulsion; modification of viscosity; prodrug or salt formation; inclusion or molecular complexes; ion exchange resins; and solid dispersion. Any method of masking the bitterness of the compound of the invention may be used.

### EXAMPLES

### The following Examples are intended to further illustrate certain embodiments of the disclosure and are not intended to limit its scope.

### Example 1

### Pharmacokinetics and Pharmacodynamics of Noribogaine in Humans

Thirty-six healthy, drug-free male volunteers, aged between 18-55 years, were enrolled in and completed the study. This was an ascending single-dose, placebo-controlled, randomized double blind, parallel group study. Mean (SD) age was 22.0 (3.3) years, mean (SD) height was 1.82 (0.08) m, and mean (SD) weight was 78.0 (9.2) kg. Twenty-six subjects were Caucasian, 3 were Asian, 1 Maori, 1 Pacific Islander, and 5 Other. The protocol for this study was approved by the Lower South Regional Ethics Committee (LRS/12/06/015), and the study was registered with the Australian New Zealand Clinical Trial Registry (ACTRN12612000821897). All subjects provided signed informed consent prior to enrolment, and were assessed as suitable to participate based on review of medical history, physical examination, safety laboratory tests, vital signs and ECG.

Within each dose level, 6 participants were randomized to receive noribogaine and 3 to receive placebo, based on a computer-generated random code. Dosing began with the lowest noribogaine dose, and subsequent cohorts received the next highest dose after the safety, tolerability, and blinded pharmacokinetics of the completed cohort were reviewed and dose-escalation approved by an independent Data Safety Monitoring Board. Blinded study drug was administered as a capsule with 240 ml of water after an overnight fast of at least 10 hours. Participants did not receive any food until at least 5 hours post-dose. Participants were confined to the study site from 12 hours prior to drug administration, until 72 hours post-dose, and there were subsequent outpatient assessments until 216 hours post-dose.

Blood was obtained for pharmacokinetic assessments pre-dose and then at 0.25, 0.5, 0.75, 1.0, 1.25, 1.5, 2, 2.5, 3, 3.5, 4, 4.5, 5, 5.5, 6, 7, 8, 10, 12, 14, 18, 24, 30, 36, 48, 60, 72, 96, 120, 168 and 216 hours post-dose. Samples were centrifuged and plasma stored at −70° C. until analyzed. Block 24 hour urine collections were obtained following study drug administration for the 30 and 60 mg cohorts. Aliquots were frozen at −20° C. until analyzed.

Pulse oximetry and capnography data were collected continuously using a GE Carescape B650 monitoring system from 2 hours prior to dosing and until six hours after dosing, and thereafter at 12, 24, 48 and 72 hours post-dosing. Additional oximetry data were collected at 120, 168 and 216 hours. Pupillary miosis was assessed by pupillometry. Dark-adapted pupil diameter was measured in triplicate using a Neuroptics PLR-200 pupillometer under standardized light intensity (<5 lux) pre-dose, and at 2, 4, 6, 12, 24, 48, 72, 96, 120, 168 and 216 hours post-dosing.

Plasma noribogaine concentrations were determined in the 3 mg and 10 mg dose groups using a validated, sensitive LCMSMS method. Sample preparation involved double extraction of basified plasma samples with tert-butyl methyl ether, drying the samples under a stream of nitrogen and reconstitution of sample with acetonitrile: B.P. water (5:95, v/v) containing 0.1% (v/v) formic acid. The compounds were separated by a 150×2.0 mm Luna 5 μm C18 column and detected with a triple—quadruple API 4000 or 5000 mass spectrometer using electrospray ionization in positive mode and multiple reaction monitoring. Noribogaine-d₄ was used as the internal standard. The precursor-product ion transition values for noribogaine were m/z 297.6→122.3, and for the internal standard noribogaine-d₄ m/z 301.1→122.2. Analyst® software was used for data acquisition and processing. The ratio of the peak area of noribogaine to the internal standard noribogaine-d₄ was used for calibration and measurement of the unknown concentration of noribogaine. The lower limit of quantification (LLOQ) was 0.025 ng/ml noribogaine. The calibration curve was between 0.025 and 25.600 ng/ml noribogaine. Mobile phase A was acetonitrile: B.P. water (5:95, v/v) containing 0.1% (v/v) formic acid, and mobile phase B was acetonitrile: B.P. water (95:5, v/v) containing 0.1% (v/v) formic acid. Total run time was 6 minutes. Binary flow: Initial concentration was 8% mobile phase B; hold at 8% mobile phase B for 0.5 minutes and linear rise to 90% mobile phase B over 1.5 minutes; hold at 90% mobile phase B for 1 minute and then drop back to 8% mobile phase B over 0.01 minute. Equilibrate system for 3 minutes. Total run time was 6 minutes. Within- and between-day assay precision was <9%, and within- and between-day assay accuracy was <9%.

Plasma noribogaine concentrations were determined in the 30 mg and 60 mg dose groups using a validated, sensitive LCMSMS method. Sample preparation involved deproteinization of plasma samples with acetonitrile and dilution of sample with 0.1% (v/v) formic acid. The compounds were separated by a 150×2.0 mm Luna 5 μm C18 column and detected with a triple—quadruple API 4000 or 5000 mass spectrometer using electrospray ionization in positive mode and multiple reaction monitoring. Noribogaine-d₄ was used as the internal standard. The precursor-product ion transition values for noribogaine were m/z 297.6→122.3, and for the internal standard noribogaine-d₄ m/z 301.1→122.2. Analyst® software was used for data acquisition and processing. The ratio of the peak area of noribogaine to the internal standard noribogaine-d₄ was used for calibration and measurement of the unknown concentration of noribogaine. The LLOQ was 0.50 ng/ml noribogaine. The calibration curve was between 0.50 and 256.00 ng/ml noribogaine. Mobile phase was the same as method A, and binary flow was also the same as method A. The within- and between-day assay precision was <9%, and the within- and between-day assay accuracy was <9%.

Plasma noribogaine glucuronide concentrations were determined in the 30 mg and 60 mg dose groups using a validated sensitive LCMSMS method. Sample preparation involved deproteinization of plasma samples with acetonitrile, drying the samples under a stream of nitrogen and reconstitution of sample with acetonitrile: B.P. water (5:95, v/v) containing 0.1% (v/v) formic acid. The compounds were separated by a 150×2.0 mm Luna 5 μm C18 column and detected with a triple—quadruple API 4000 or 5000 mass spectrometer using electrospray ionization in positive mode and multiple reaction monitoring. Noribogaine-d₄ was used as the internal standard. The precursor-product ion transition values for noribogaine glucuronide were m/z 472.8→297.3, and for the internal standard noribogaine-d₄ m/z 301.1→122.2. Analyst® software was used for data acquisition and processing. The ratio of the peak area of noribogaine glucuronide to the internal standard noribogaine-d₄ was used for calibration and measurement of the unknown concentration of noribogaine glucuronide. The LLOQ was 0.050 ng/ml noribogaine glucuronide. The calibration curve was between 0.050 and 6.400 ng/ml noribogaine glucuronide. Mobile phases was the same as method A. Binary flow: Initial concentration was 6% mobile phase B; hold at 6% mobile phase B for 0.5 minutes and linear rise to 90% mobile phase B over 2 minutes; hold at 90% mobile phase B for 1 minute and then drop back to 6% mobile phase B over 0.01 minute. Equilibrate system for 3.5 minutes. Total run time was 7 minutes. The within- and between-day assay precision was <11%, and the within- and between-day assay accuracy was <10%.

Urine noribogaine and noribogaine glucuronide concentrations were determined in the 30 mg and 60 mg dose groups using a validated sensitive LCMSMS method. Sample preparation involved deproteinization of urine samples with acetonitrile and dilution of the sample with 0.1% (v/v) formic acid. The compounds were separated by a 150×2.0 mm Luna 5 μm C18 column and detected with a triple—quadruple API 4000 or 5000 mass spectrometer using electrospray ionization in positive mode and multiple reaction monitoring. Noribogaine-d₄ was used as the internal standard. The precursor-product ion transition values for noribogaine were m/z 297.6→122.3, noribogaine glucuronide m/z 472.8→297.3, and for the internal standard noribogaine-d₄ m/z 301.1→122.2. Analyst® software was used for data acquisition and processing. The ratios of the peak area of noribogaine and noribogaine glucuronide to the internal standard noribogaine-d₄ were used for calibration and measurement of the unknown concentration of noribogaine and its glucuronide. Assay LLOQ was 20.0 ng/ml for noribogaine and 2.0 ng/ml for noribogaine glucuronide. The calibration curve was between 20.0 and 5120.0 ng/ml noribogaine, and 2.0 and 512.0 ng/ml noribogaine glucuronide. Mobile phases were as described in method A, and binary flow as in method C. The within- and between-day assay precision was <13%, and within- and between-day assay accuracy was <12%.

Noribogaine and noribogaine glucuronide concentrations above the limit of quantification were used to calculate pharmacokinetic parameters using model-independent methods. The maximum plasma concentration (Cmax) and time to maximum plasma concentration (Tmax) were the observed values. Plasma concentration data in the post-distribution phase of the plasma concentration-time plot were fitted using linear regression to the formula ln C=ln Co−t.Kel, where Co was the zero-time intercept of the extrapolated terminal phase and Kel was the terminal elimination rate constant. The half-life (t_{1/2} ) was determined using the formula t_{1/2}=0.693/Kel. The area under the concentration-time curve (AUC) from time zero to the last determined concentration-time point (tf) in the post distribution phase was calculated using the trapezoidal rule. The area under the curve from the last concentration-time point in the post distribution phase (Ctf) to time infinity was calculated from AUC_{t-∞}=Ctf/Kel. The concentration used for Ctf was the last determined value above the LLOQ at the time point. The total AUC_{0-∞}, was obtained by adding AUC_{tf} and AUC_{t-∞}. Noribogaine apparent clearance (CL/F) was determined using the formula CL/F=Dose/AUC_{0-∞}×1000, and apparent volume of distribution (Vd/F) was determined using the formula Vd/F=(CL/F)/Kel. Total urine noribogaine was the sum of both analytes.

Summary statistics (means, standard deviations, and coefficients of variation) were determined for each dose group for safety laboratory test data, ECG and pharmacokinetic parameters, and pharmacodynamic variables. Categorical variables were analysed using counts and percentages. Dose-proportionality of AUC and Cmax was assessed using linear regression. The effect of dose on pharmacodynamic parameter values over time was assessed using two-factor analysis of variance (ANOVA). Pairwise comparisons (with Tukey-Kramer adjustment) between each dose group to the placebo were conducted at each time point using the least squares estimates obtained from the ANOVA, using SAS Proc Mixed (SAS ver 6.0).

### Results

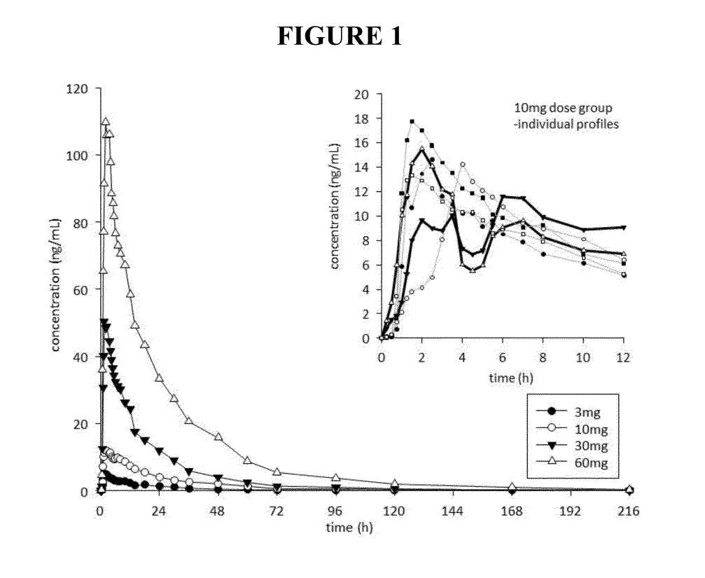

Pharmacokinetics: Mean plasma concentration-time plots of noribogaine are shown in FIG. 1, and mean pharmacokinetic parameters are shown in Table 1.

TABLE 1

| | | | |
|---|---|---|---|
| 3 mg (n = 6) | 10 mg (n = 6) | 30 mg (n = 6) | 60 mg (n = 6) |
| (mean (SD)) | (mean (SD)) | (mean (SD)) | (mean (SD) |

Noribogaine

| | | | | |
|---|---|---|---|---|
| AUC_{0-∞} | 74.2 (13.1) | 254.5 (78.9) | 700.4 (223.3) | 1962.2 (726.5) |
| (ng · hr/ml) | | | | |
| AUC₀₋₂₁₆ | 72.2 (13.2) | 251.4 (78.5) | 677.6 (221.1) | 1935.4 (725.4) |
| (ng · hr/ml) | | | | |
| Cmax | 5.2 (1.4) | 14.5 (2.1) | 55.9 (14.8) | 116.0 (22.5) |
| (ng/ml) | | | | |
| Tmax (hr) | 1.9 (0.6) | 2.9 (1.8) | 1.8 (0.6) | 2.4 (0.6) |
| t_{1/2} (hr) | 40.9 (8.7) | 49.2 (11.5) | 27.6 (7.0)) | 29.1 (9.3) |
| Vd/F (L) | 2485.1 (801.5) | 3085.8 (1197.0) | 1850.8 (707.9) | 1416.8 (670.1) |
| CL/F (L/h) | 41.4 (7.0) | 42.3 (12.0) | 46.9 (16.4) | 34.0 (11.4) |

Noribogaine glucuronide

| | | | | |
|---|---|---|---|---|
| AUC_{0-∞} | — | — | 25.8 (9.3) | 67.1 (21.9) |
| (ng · hr/ml) | | | | |
| AUC₀₋₂₁₆ | — | — | 25.7 (9.1) | 65.0 (21.5) |
| (ng · hr/ml) | | | | |
| Cmax | — | — | 1.8 (0.6) | 4.1 (1.2) |
| (ng/ml) | | | | |
| Tmax (hr) | — | — | 3.0 (0.6) | 3.8 (1.2) |
| t_{1/2} (hr) | — | — | 20.6 (4.9) | 23.1 (3.0) |

Noribogaine was rapidly absorbed, with peak concentrations occurring 2-3 hours after oral dosing. Fluctuations in individual distribution-phase concentration-time profiles may suggest the possibility of enterohepatic recirculation (see highlighted individual 4-8 hour profiles in FIG. 1, insert). Both Cmax and AUC increased linearly with dose (Table 1, upper panel). Mean half-life estimates of 28-50 hours were observed across dose groups for noribogaine. Volume of distribution was extensive (1417-3086 L across dose groups).

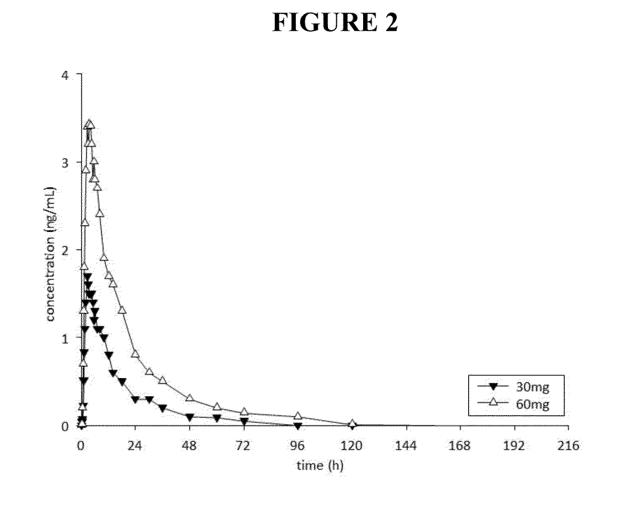

Mean plasma noribogaine glucuronide concentration-time plots for the 30 mg and 60 mg dose group are shown in FIG. 2, and mean pharmacokinetic parameters are shown in Table 1, lower panel. Noribogaine glucuronide was detected in all subjects by 0.75 hours, with peak concentrations occurring 3-4 hours after noribogaine dosing. Mean half-life of 21-23 hours was estimated for plasma noribogaine glucuronide. The proportion of noribogaine glucuronide Cmax and AUC relative to noribogaine was 3-4% for both dose groups. Total urine noribogaine elimination was 1.16 mg and 0.82 mg for the 30 mg and 60 mg dose groups respectively, representing 3.9% and 1.4% of the doses administered.

The subject mean serum levels over time of noribogaine free base from a single dose of 3 mg noribogaine free base under fasting conditions were plotted. The mean Cₘₐₓ of 5.2 ng/ml was observed 1.9 hours after administration, while the mean AUC/24 hr of 3.1 ng/ml was obtained.

The subject mean serum levels over time of noribogaine free base from a single dose of 10 mg noribogaine free base under fasting conditions were plotted. The mean Cₘₐₓ of 14.5 ng/ml was observed 2.9 hours after administration, while the mean AUC/24 hr of 10.6 ng/ml was obtained.

The subject mean serum levels over time of noribogaine free base from a single dose of 30 mg noribogaine free base under fasting conditions were plotted. The mean Cₘₐₓ of 55.9 ng/ml was observed between 1.75 hours after administration, while the mean AUC/24 of 29.2 ng/ml was obtained.

The subject mean serum levels over time of noribogaine free base from a single dose of 60 mg noribogaine free base under fasting conditions were plotted. The mean Cₘₐₓ of 116 ng/ml was observed between 1.75 hours after administration, while the mean AUC/24 ng/ml of 61 was obtained.

The subject mean serum levels over time of noribogaine free base for all 4 cohorts were plotted. The extrapolated dosage of noribogaine free base required to provide a Cₘₐₓ ranging from about 5.2 ng/ml to about 1980 ng/ml and an AUC/24 hr of about 3.1 ng/ml to about 1100 ng/ml was determined.

Pharmacodynamics: There was no evidence of pupillary constriction in subjects dosed with noribogaine. No between-dose group differences in pupil diameter were detected over time. After adjusting for baseline differences, comparison of each dose group with placebo by ANOVA showed no statistically significant differences (p>0.9).

Noribogaine treatment showed no analgesic effect in the cold pressor test. Analgesic effect was assessed based on duration of hand immersion in ice water and on visual analog scale (VAS) pain scores upon hand removal from the water bath. For duration of hand immersion, after adjusting for baseline differences, comparison of each dose group with placebo by ANOVA showed no statistically significant differences (p>0.9). Similarly, for VAS pain scores, after adjusting for baseline differences, comparison of each dose group with placebo by ANOVA showed no statistically significant differences (p=0.17).

### Example 2

### Safety and Tolerability of Noribogaine in Healthy Humans

Safety and tolerability of noribogaine were tested in the group of volunteers from Example 1. Cold pressor testing was conducted in 1° C. water according to the method of Mitchell et al. (J. Pain 5:233-237, 2004) pre-dose, 6, 24, 48, 72 and 216 hours post-dosing. Safety evaluations included clinical monitoring, recording of adverse events (AEs), safety laboratory tests, vital signs, ECG telemetry from −2 h to 6 h after dosing, and 12-lead electrocardiograms (ECGs) up to 216 hours post-dosing.

### Results

A total of thirteen adverse events were reported by seven participants (Table 2). Six adverse events were reported by three participants in the placebo group, five adverse events were reported by two subjects in the 3 mg dose group, and one adverse event was reported by single subjects in the 10 mg and 30 mg dose groups, respectively. The most common adverse events were headache (four reports) and epistaxis (two reports). All adverse events were of mild-moderate intensity, and all resolved prior to study completion. There were no changes in vital signs or safety laboratory tests of note. In particular, there were no changes in oximetry or capnography, or changes in respiratory rate. There were no QTcF values >500 msec at any time. One subject dosed with 10 mg noribogaine had a single increase in QTcF of >60 msec at 24 hours post-dosing.

TABLE 2

| Dose (mg) | Mild | Moderate | Severe |
|---|---|---|---|
| Placebo | Blepharitis | Epistaxis | — |
| | Bruising | | |
| | Dry Skin | | |
| | Eye pain, nonspecific | | |
| | Infection at cannula site | | |
| 3 | Back pain | Headache | — |
| | Dizziness | | |
| | Epistaxis | | |
| | Headache | | |
| 10 | Headache | — | — |
| 30 | Headache | — | — |
| 60 | — | — | — |

### Example 3

### Safety, Tolerability, and Efficacy of Noribogaine in Opioid-Addicted Humans

This example is to illustrate that noribogaine can be administered at a therapeutic dosing while maintaining an acceptable QT interval. While the therapy employed is directed to opioid-dependent participants in a randomized, placebo-controlled, double-blind trial, the results show that a therapeutic window can be established for noribogaine.

The efficacy of noribogaine in humans was evaluated in opioid-dependent participants in a randomized, placebo-controlled, double-blind trial. Patients had been receiving methadone treatment as the opioid substitution therapy, but were transferred to morphine treatment prior to noribogaine administration. This was done to avoid negative noribogaine-methadone interactions that are not observed between noribogaine and morphine. See U.S. application Ser. No. 14/214,157, filed Mar. 14, 2014 and Ser. No. 14/346,655, filed Mar. 21, 2014, which are incorporated herein by reference in their entireties.

Three cohorts of nine (9) subjects (6 administered noribogaine and 3 administered placebo in each cohort) were evaluated for tolerability, pharmacokinetics, and efficacy. Cohort 1 received a single dose of 60 mg noribogaine or placebo. Cohort 2 received a single dose of 120 mg noribogaine or placebo. Cohort 3 received a single dose of 180 mg noribogaine or placebo. Treatment was administered 2 hours after last morphine dose and the time to resumption of morphine (opioid substitution treatment, OST) was determined. Few adverse effects of noribogaine were observed in any of the participants, including no hallucinatory effects. Table 3 shows the reported adverse events for each treatment that were not attributable to withdrawal from opioids. Headaches were frequent in the placebo and 60 mg noribogaine treatment groups, but were attenuated in the 120 mg and 180 mg dose groups.

TABLE 3
Treatment Emergent Adverse Events Summary

| System Organ Class | | Placebo | 60 mg | 120 mg | 180 mg |
|---|---|---|---|---|---|
| Preferred Term | | (N = 9) | (N = 6) | (N = 6) | (N = 6) |
| Number of Subjects Reporting | | 19:7 (77.8%) | 15:5 (83.3%) | 28:6 (100.0%) | 17:4 (66.7%) |
| any AEs | | | | | |
| Ear and Labyrinth Disorders | | 0 | 0 | 2:2 (33.3%) | 0 |
| | Tinnitus | 0 | 0 | 2:2 (33.3%) | 0 |
| Eye Disorders | | 2:2 (22.2%) | 3:3 (50.0%) | 5:5 (83.3%) | 5:4 (66.7%) |
| | Visual Impairment | 2:2 (22.2%) | 2:2 (33.3%) | 5:5 (83.3%) | 5:4 (66.7%) |
| | Dry Eye | 0 | 1:1 (16.7%) | 0 | 0 |
| Gastrointestinal Disorders | | 3:2 (22.2%) | 2:2 (33.3%) | 7:2 (33.3%) | 4:2 (33.3%) |
| | Nausea | 1:1 (11.1%) | 0 | 3:2 (33.3%) | 2:2 (33.3%) |
| | Dry Mouth | 0 | 0 | 1:1 (16.7%) | 1:1 (16.7%) |
| | Vomiting | 0 | 0 | 2:1 (16.7%) | 1:1 (16.7%) |
| | Diarrhoea | 1:1 (11.1%) | 0 | 1:1 (16.7%) | 0 |
| | Dyspepsia | 1:1 (11.1%) | 2:2 (33.3%) | 0 | 0 |
| General Disorders and Administration | | 4:3 (33.3%) | 0 | 2:2 (33.3%) | 1:1 (16.7%) |
| Site Conditions | | | | | |
| | Catheter Site Related Reaction | 0 | 0 | 0 | 1:1 (16.7%) |
| | Catheter Site Pain | 3:2 (22.2%) | 0 | 2:2 (33.3%) | 0 |
| | Malaise | 1:1 (11.1%) | 0 | 0 | 0 |
| Infections and Infestations | | 1:1 (11.1%) | 0 | 1:1 (16.7%) | 2:2 (33.3%) |
| | Cellulitis | 0 | 0 | 1:1 (16.7%) | 1:1 (16.7%) |
| | Urinary Tract Infection | 0 | 0 | 0 | 1:1 (16.7%) |
| | Catheter Site Infection | 1:1 (11.1%) | 0 | 0 | 0 |
| Musculoskeletal and Connective | | 1:1 (11.1%) | 2:1 (16.7%) | 0 | 2:2 (33.3%) |
| Tissue Disorders | | | | | |
| | Back Pain | 1:1 (11.1%) | 2:1 (16.7%) | 0 | 1:1 (16.7%) |
| | Limb Discomfort | 0 | 0 | 0 | 1:1 (16.7%) |
| Nervous System Disorders | | 7:5 (55.6%) | 7:4 (66.7%) | 5:4 (66.7%) | 3:2 (33.3%) |
| | Headache | 6:5 (55.6%) | 7:4 (66.7%) | 2:2 (33.3%) | 3:2 (33.3%) |
| | Hyperaesthesia | 0 | 0 | 1:1 (16.7%) | 0 |
| | Pseudoparalysis | 0 | 0 | 1:1 (16.7%) | 0 |
| | Tremor | 0 | 0 | 1:1 (16.7%) | 0 |
| | Somnoience | 1:1 (11.1%) | 0 | 0 | 0 |
| Psychiatric Disorders | | 1:1 (11.1%) | 1:1 (16.7%) | 0 | 0 |
| | Depressed Mood | 0 | 1:1 (16.7%) | 0 | 0 |
| | Euphoric Mood | 1:1 (11.1%) | 0 | 0 | 0 |
| Respiratory, Thoracic and | | 0 | 0 | 4:2 (33.3%) | 0 |
| Mediastinal Disorders | | | | | |
| | Epistaxis | 0 | 0 | 2:1 (16.7%) | 0 |
| | Oropharyngeal Pain | 0 | 0 | 1:1 (16.7%) | 0 |
| | Rhinorrhoea | 0 | 0 | 1:1 (16.7%) | 0 |
| Skin and Subcutaneous | | 0 | 0 | 2:1 (16.7%) | 0 |
| Tissue Disorders | | | | | |
| | Skin Discomfort | 0 | 0 | 1:1 (16.7%) | 0 |
| | Skin Irritation | 0 | 0 | 1:1 (16.7%) | 0 |

Note:
Within each system organ class, Preferred Terms are presented by descending incidence of descending dosages groups and then the placebo group.
Note:
N = number of subjects in the safety population.

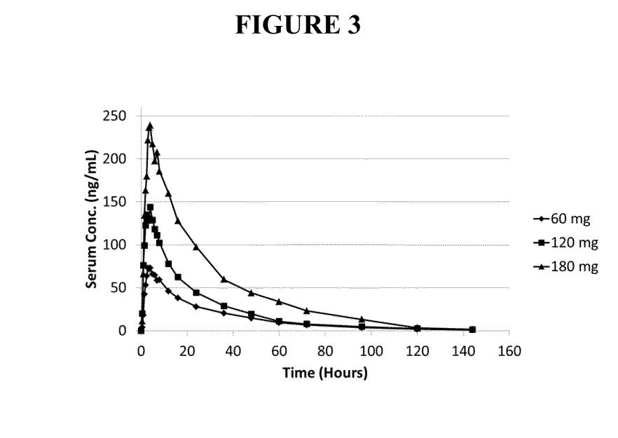

FIG. 3 indicates the average serum noribogaine concentration over time after administration of noribogaine for each cohort (60 mg, diamonds; 120 mg, squares; or 180 mg, triangles). Further results are detailed in U.S. Provisional Patent Application No. 62/023,100, filed Jul. 10, 2014, and titled “METHODS FOR ACUTE AND LONG-TERM TREATMENT OF DRUG ADDICTION,” which is incorporated herein by reference in its entirety.

### Results

Pharmacokinetic results for each cohort are given in Table 4. Maximum serum concentration of noribogaine (Cmax) increased in a dose-dependent manner. Time to Cmax (Tmax) was similar in all three cohorts. Mean half-life of serum noribogaine was similar to that observed in healthy patients.

TABLE 4
Pharmacokinetic results from the Patients in Phase IB Study

| | Cohort 1 (60 mg) | Cohort 2 (120 mg) | Cohort 3 (180 mg) |
|---|---|---|---|
| | Data (mean ± SD) | Data (mean ± SD) | Data (mean ± SD) |
| PK parameter | [range] | [range] | [range] |
| Cmax | 81.64 ± 23.77 | 172.79 ± 30.73 | 267.88 ± 46.92 |
| (ng/ml) | [41.29-113.21] | [138.84-229.55] | [204.85-338.21] |
| Tmax | 3.59 ± 0.92 | 2.99 ± 1.23 | 4.41 ± 1.80 |
| (hours) | [2.50-5.00] | [0.98-4.02] | [3.00-8.00] |
| AUC_{(0-T)} | 2018.01 ± 613.91 | 3226.38 ± 1544.26 | 6523.28 ± 2909.80 |
| (ng · hr/ml) | [1094.46-2533.44] | [1559.37-5638.98] | [3716.69-10353.12] |
| AUC_{(0-¥)} | 2060.31 ± 609.39 | 3280.50 ± 1581.43 | 6887.67 ± 3488.91 |
| (ng · hr/ml) | [1122.29-2551.63] | [1595.84-5768.52] | [3734.21-12280.91] |
| Half-life | 29.32 ± 7.28 | 30.45 ± 9.14 | 23.94 ± 5.54 |
| (hrs) | [18.26-37.33] | [21.85-48.33] | [19.32-34.90] |
| Vd/F | 1440.7 ± 854.0 | 2106.43 ± 1644.54 | 1032.19 ± 365.30 |
| | [619.5-2772.5] | [824.24-5243.78] | [581.18-1608.98] |
| Cl/F | 32.14 ± 12.38 | 44.68 ± 21.40 | 31.47 ± 13.12 |
| | [23.51-53.46] | [20.80-75.20] | [14.66-48.20] |

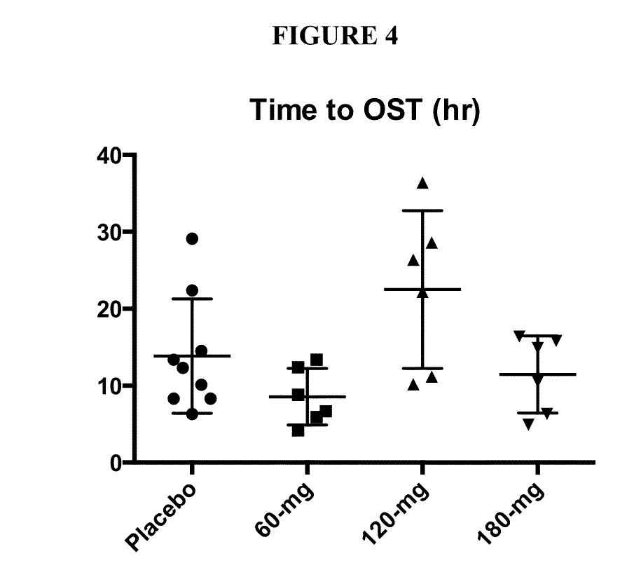

FIG. 4 indicates the time to resumption of morphine (OST) for patients treated with placebo (circles), 60 mg noribogaine (squares), 120 mg noribogaine (triangles), and 180 mg noribogaine (inverted triangles). Patients receiving a single 120 mg dose of noribogaine exhibited an average time to resumption of opioids of greater than 20 hours. Patients receiving a single 180 mg dose of noribogaine exhibited an average time to resumption of opioids similar to that of placebo. This demonstrates that increasing the dose of noribogaine to 180 mg results in a shorter time to resumption of OST than observed in patients receiving 120 mg noribogaine. Time to resumption of OST after treatment with 180 mg was still longer than untreated patients (7 hours, not shown) or those administered 60 mg noribogaine.

Patients were evaluated based on the Clinical Opiate Withdrawal Scale (COWS), Subjective Opiate Withdrawal Scale (SOWS), and Objective Opiate Withdrawal Scale (OOWS) scoring systems over the period of time between administration of noribogaine (or placebo) until resumption of OST. These scales are outlined in Guidelines for the Psychosocially Assisted Pharmacological Treatment of Opioid Dependence, World Health Organization, Geneva (2009), Annex 10, which is incorporated herein by reference in its entirety. The scales measure the intensity of withdrawal symptoms, based on clinical, subjective, and objective indicia.

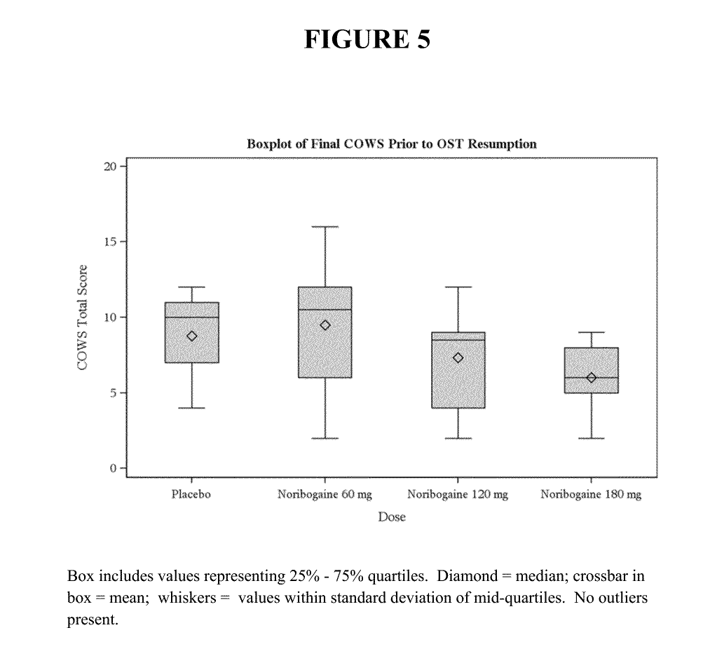

FIG. 5 shows the COWS scores at time of resumption of OST for each cohort. Box includes values representing 25%-75% quartiles. Diamond=median; crossbar in box=mean; whiskers=values within standard deviation of mid-quartiles. No outliers present. The highly variable COWS scores across and within each cohort indicates that patients were resuming opiates without relation to the intensity of withdrawal. This was also reflected in SOWS and OOWS scores at the time of resumption of OST.

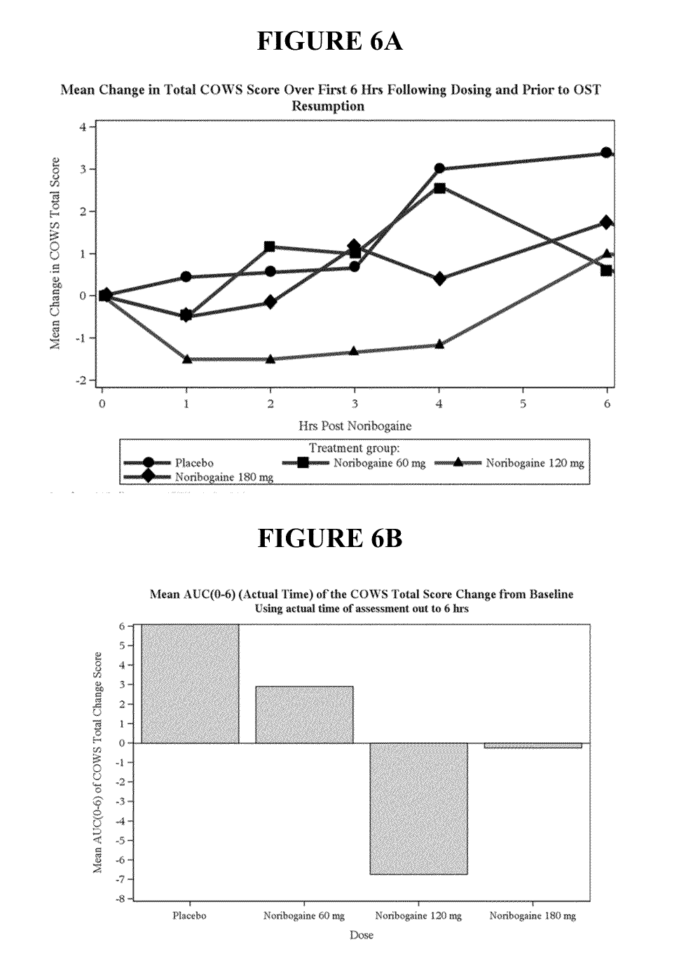

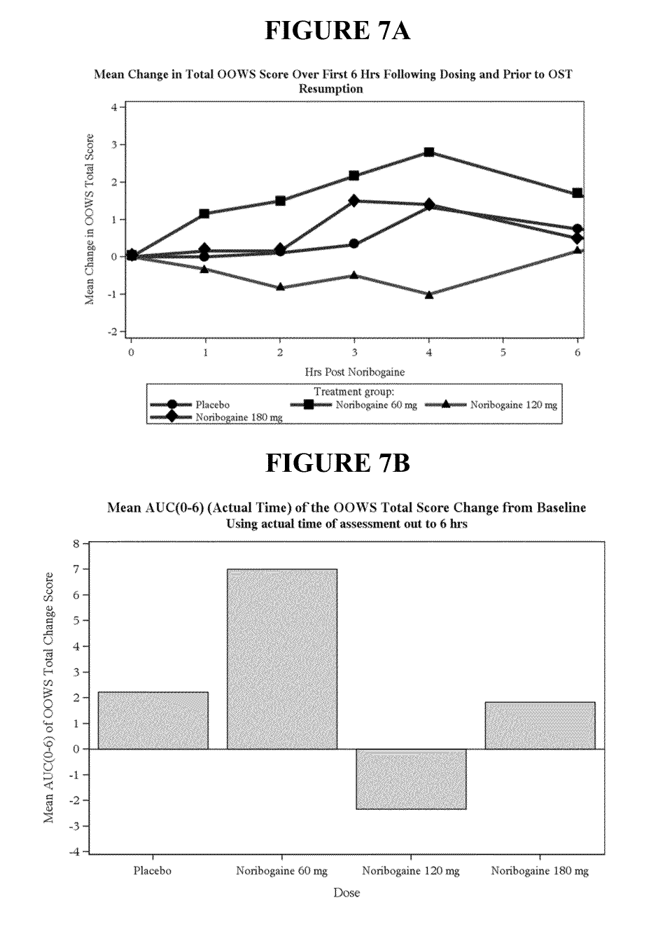

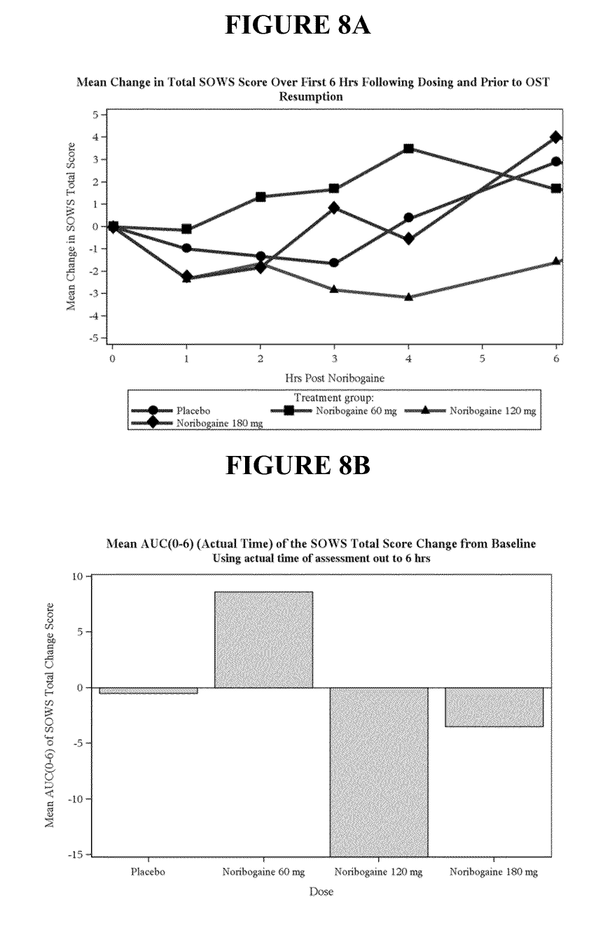

FIG. 6A shows the mean change in total COWS scores over the first six hours following dosing and prior to resumption of OST. FIG. 6B shows the mean AUC(0-6 hours) of the COWS total score change from baseline. FIG. 7A shows the mean change in total OOWS scores over the first six hours following dosing and prior to resumption of OST. FIG. 7B shows the mean AUC(0-6 hours) of the OOWS total score change from baseline. FIG. 8A shows the mean change in total SOWS scores over the first six hours following dosing and prior to resumption of OST. FIG. 8B shows the mean AUC(0-6 hours) of the SOWS total score change from baseline. These data indicate that withdrawal symptoms get worse over time after cessation of OST, and that patients administered placebo experience generally worse withdrawal symptoms over that period. Patients who received 120 mg noribogaine generally experienced fewer withdrawal symptoms than the other patients, regardless of the scale used. Patients administered placebo generally experienced more withdrawal symptoms than patients who were administered noribogaine.

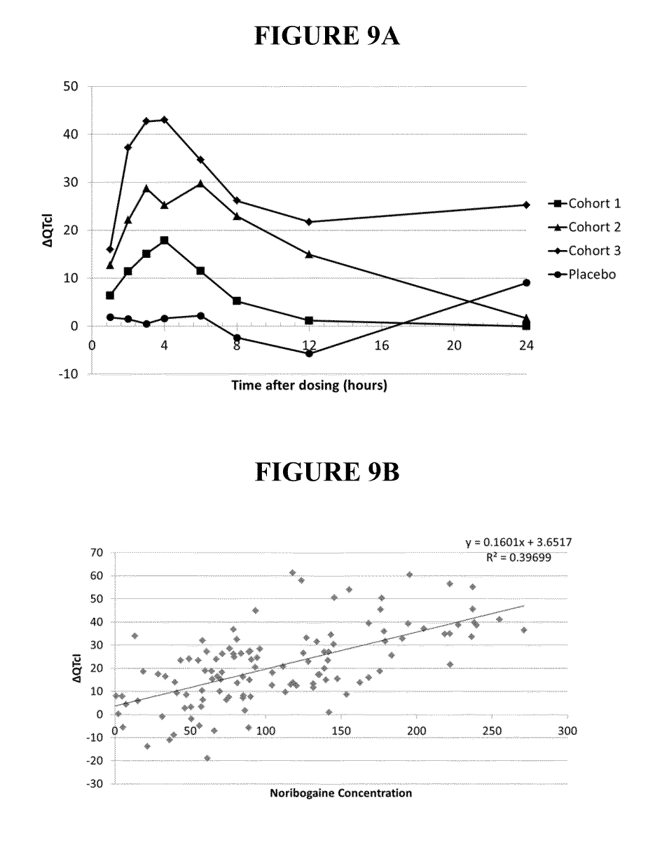

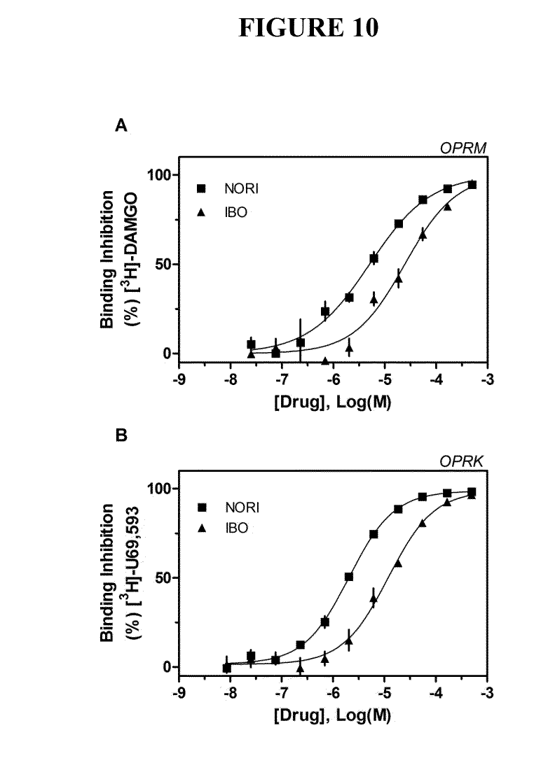

Patients' QT intervals were evaluated at regular time points throughout the study. FIG. 9A shows the average change in QT interval (ΔQTcl, i.e., QT interval prolongation) over the first 24 hours post noribogaine (or placebo) administration. FIG. 9B shows the estimated correlation between noribogaine concentration and change in QT interval. There is a dose-dependent increase in QT interval prolongation that is correlated with the serum concentration of noribogaine.

Based on above data, it is believed that the therapeutic window for a single bolus dose of noribogaine is bound at the lower end by 50 mg and at the upper end by less than 180 mg. In particular, the therapeutic serum concentration in vivo appears to be between about 50 ng/mL and about 180 ng/mL.

### Example 4

### Efficacy of Noribogaine to Modulate Opioid Tolerance in Humans

A female patient, age 59, undergoing opioid analgesic therapy for chronic back pain, is treated with noribogaine hydrochloride at a dose of about 2 mg/kg concurrently with the opioid. The amount of opioid required to treat her back pain to the same level as before noribogaine treatment is determined after noribogaine treatment.

### Example 5

### Noribogaine is a Mixed Agonist/Antagonist Opioid Ligand with Functional Selectivity

Materials: [Phenyl-3,4-³H]-U-69,593 (43.6 Ci/mmol), [Tyrosyl-3,5-³H(N)]-DAMGO ([D-Ala², N-MePhe⁴, Gly⁵-ol]-enkephalin) (50 Ci/mmol) and [35S]GTPγS (Guanosine 5″-(gamma-thio)triphosphate) (1250 Ci/mmol) were purchased from PerkinElmer Life Sciences (Boston, Mass.). U69,593, naloxone, nor-binaltorphimine (nor-BNI), morphine, nalmefene, dynorphin A, DAMGO ([D-Ala2, NMe-Phe4, Gly-ol5]-enkephalin), GTPγS, GDP and all buffer constituents were purchased from Sigma-Aldrich Corp. (St. Louis, Mo.). CHO—K1 cell lines expressing human opioid receptors were provided by Dr. Toll at Torrey Pines Institute. Ibogaine (PubChem CID: 363272) was provided by Dr. Mash at the University of Miami (Miami, Fla.). 18-methoxycoronaridine (18-MC, PubMed CID: 15479177) was purchased at Orbiter Pharmaceutical. Noribogaine hydrochloride (PubChem CID: 457966) was purchased at Sigma Aldrich Chemie GmbH (Buchs, Switzerland).

Membrane preparation: Membranes from rat midbrain tissues were purchased at Chantest (Cleveland, Ohio). Membranes of human OPRK were purchased from PerkinElmer Life Sciences (Boston, Mass.) and human OPRM CHO—K1 cells were prepared as described below. Adherent cells were harvested on ice, with cold PBS and a cell scraper, pelleted and frozen at −80° C. overnight. Cell lysis was performed at 4° C. in 50 mM Tris (pH 7), 2.5 mM EDTA and cOmplete protease inhibitor cocktail (cOmplete, F. Hoffmann-La Roche Ltd). Cells were homogenized with a polytron and centrifuged at 2500 rpm for 10 minutes at 4° C. Supernatant was recovered and the process was repeated once. Supernatant was centrifuged at 21,000 rpm for 90 minutes at 4° C. and pellets were re-suspended in 50 mM Tris (pH 7) and 0.32 M sucrose. Total protein concentration was evaluated using a Thermo Scientific NanoDrop spectrophotometer and by Bradford assay Membrane sample aliquots were stored at −80° C. at 1 to 5 mg/mL protein concentration. Membranes from brain tissues were stored in 50 mM Tris (pH 7), 1 mM EDTA and 0.32 M sucrose with protease inhibitors cocktail.

Radioligand Binding: Competitive binding experiments were performed using Perkin Elmer recommended conditions. Membranes were thawed on ice and diluted in binding buffer 50 mM Tris-HCl pH 7.4, 5 mM MgCl₂ at 5 μg of membrane per reaction. Competition binding assay experiment were performed in 500 μL total volume containing [³H]U69,593 (0.88 nM) for OPRK membranes or [³H]DAMGO (0.75 nM) for OPRM membranes in the presence of increasing concentrations of each unlabeled drug (noribogaine, ibogaine, 18-MC, U69,593, morphine, DAMGO, naloxone) for 60 minutes at 25° C. Nonspecific binding was defined in the presence of 1 μM naloxone. Bound and free radiolabelled ligands were separated by filtration using a MicroBeta FilterMate-96 Harvester and wash 6×1 mL with ice cold wash buffer (50 mM Tris-HCl pH 7.4) over GF/B filter (presoaked in 0.5% BSA) (Perkin Elmer, Waltham, Mass.). Radioactivity counts were determined using Perkin Elmer Micro βeta microplate counter with scintillation cocktail MicroScint-20™ according to manufacturer recommendations. Data were collected and the half maximal inhibitory concentration (IC₅₀) and apparent binding affinity (Kᵢ) for all data sets were calculated with GraphPad Prism 5.04.

[³⁵ S]GTPγS Binding Assay: [³⁵S]GTPγS binding to Gα proteins was determined using a modified procedure from (Toll, Berzetei-Gurske et al. 1998). Cell membranes were thawed on ice and experiments were carried out in a 96-well format. Cell membranes (10 μg per reaction) were incubated in a binding buffer (20 mM HEPES, pH 7.4, 100 mM NaCl, 10 mM MgCl₂×6H₂O, 0.2% bovine serum albumin, and GDP 10 μM, pH 7.4) containing 80 pM [³⁵S]GTPγS and varying concentrations of opioid agonists (U69,593, DAMGO, morphine, dynorphin A, nalmefene, or noribogaine) in a total volume of 100 μL for 60 minutes at 25° C. Membranes were pre-incubated with the GDP for 15 minutes on ice prior to the addition of ligands. Antagonists were added to the membrane solution 20 minutes prior the addition of the agonist, and [³⁵S]GTPγS was added 5 minutes after the agonist. Non-specific and basal levels of GTPγS binding was evaluated by using 10 μM cold GTPγS and binding buffer, respectively. Bound and free [³⁵S]GTPγS were separated by filtration using a MicroBeta FilterMate-96 Harvester and wash 4 ×1 mL with ice cold wash buffer (20 mM Tris, pH 7.4, and 2,5 mM MgCl₂×6H₂O, pH 7.4) over GF/B filter (presoaked in 0.5% BSA) (Perkin Elmer, Waltham, Mass.). Radioactivity counts were determined using Perkin Elmer Micro βeta microplate counter with scintillation cocktail MicroScint-20™ according to manufacturer recommendations. Data were collected and the half maximal effective concentration (EC₅₀) and maximal responses (Eₘₐₓ) values were calculated with GraphPad Prism 5.04.

β-arrestin-2 recruitment Assay: The PathHunter enzyme complementation Arrestin-2 Recruitment assay was performed at DiscoveRx Corporation, Fremont, Calif.. This assay utilized CHO—K1 cells stably transfected to overexpress β-arrestin-2 fused to a β-galactosidase fragment together with human OPRK gene (NM_{—}000912.3, human KOR) or human OPRM gene (NM_{—}000914.3, encoding human MOR). Briefly, when β-arrestin-2 travels to active receptor, the complementary β-galactosidase fragments fused to the receptor and β-arrestin interact to form a functional enzyme with activity that is detected by chemiluminescence. For all in vitro assays, data were normalized as a percentage of control agonist responses, typically defined by dynorphin A stimulated activity in the OPRK assays, and [Met] Enkephalin stimulated activity in the OPRM assays. For agonist dose-response experiments, cells were treated with test compound for 90 minutes prior to assessment of enzyme complementation. For antagonist dose-inhibition experiments, the cells were incubated with the test compound for 30 minutes prior to agonist addition. For OPRK, a dose corresponding to the EC₈₀ (316.9 nM) of Dynorphin A was used. For OPRM a dose corresponding to the EC₈₀ (2.1 μM) of [Met] Enkephalin was used.

Data Analysis: The IC₅₀ and Kᵢ values for ligands in the radioactive binding assays were determined by fitting competition binding data of individual experiments normalized to buffer (total binding) and 1 μM naloxone (nonspecific binding) to a single site competition model in GraphPad Prism 5.04 using the transformation of Cheng and Prusoff (CFeq): Kᵢ=IC₅₀/(1+[S]/Kₘ), where [S] is the concentration of agonist and Kₘ is the Kᵢ value for [³H]U69,593 and [³H]DAMGO determined by homologous competition. The EC₅₀ and Eₘₐₓ values to agonists for [³⁵S]GTPγS binding and β-arrestin-2 translocation were determined by fitting data from individual experiments to sigmoidal concentration-response curves with variable slope in GraphPad Prism 5.04. Final mean and S.E.M. were calculated using individual values from each experiment. Functional inhibitory potency (Kₑ) values for agonist dose-response displacement experiments were calculated using the Gaddum/Schild EC₅₀ shift calculation or with the following equation: Kₑ=[nanomolar antagonist]/(dose ratio-1), where dose ratio is the ratio of the EC₅₀ for an agonist in the presence and absence of another ligand/inhibitor at a given concentration. Kₑ values for dose-inhibition experiments were calculated with a modified CFeq: Kₑ=IC₅₀/(1+[S]/EC₅₀) where [S] is the concentration of agonist used and EC₅₀ is the functional potency of the agonist.

Coupling efficiency (e-coupling) values indicated the relationship between the apparent binding affinity Kᵢ versus the apparent functional potency EC₅₀ of a given agonist ligand and used the equation pKi-pEC₅₀. For the functional inhibitory components of antagonists and partial agonists, e-coupling represents the relationship between the Kᵢ versus the Kₑ of a given ligand (against Dynorphin A for OPRK assays, and against U69,593 for OPRM assays) and used the equation pKᵢ-pKₑ. Efficacy efficiency (e-signal) values indicated the ratio of the Eₘₐₓ to a given agonist ligand versus the Eₘₐₓ to Dynorphin A (or U69,593) and uses the equation Eₘₐₓ(control agonist)/Eₘₐₓ(test compound). For inhibitory ligands, e-signal was calculated using maximal level of inhibition (Iₘₐₓ) normalized from 0 (basal, buffer) to 1 (agonist without inhibitor). Bias-coupling (quantification of pathway bias) was evaluated by subtracting the EC₅₀ or the Kₑ issued from the G-protein pathway assays by those issued from β-arrestin pathway assays for a given ligand and in a linear (nM) scale. Bias-efficacy in favor of the G-protein pathway was evaluated by dividing the functional activation and the functional inhibition maximum responses (e-signal) from the G-protein pathway by the beta-arrestin pathway assays for a given ligand.

μ-Opioid Receptor Ibogaine binding model: The mouse μ-opioid receptor OPRM co-crystal structure available in the Protein Data Bank (PDB), PDB accession 4dkl, Uniprot accession P42866, was used. The mouse OPRM has 94% (global) sequence identity to the corresponding human receptor (Uniprot accession P35372) and all residues in the binding site are identical. The receptor was crystallized as a fusion protein (OPRM—T4L) with an irreversible morphine antagonist ligand (bound to Lys233, pdb numbering). All simulations were performed using the Schrodinger 2014.2 and Desmond 2014.2 software suite. For initial docking studies, the PDB file into Maestro 9.5 (Schrodinger) was imported and the standard protein preparation workflow to assign bond orders and clean up the structure including hydrogen bond optimization and constrained minimization was run. In the preparation process missing side chains were added using Prime. The fusion protein was manually cut and removed between residues Val262 and Glu270 to leave just the GPCR (G protein-coupled receptor) transmembrane domain; the cut residues were capped as primary amide (C-terminal) and acetate (N-terminal). A (non-covalent) ligand entry (separate from the chain) was manually created in Maestro. The resulting protein complex was again processed via the protein preparation workflow. A docking grid was created around the co-crystal ligand using Glide (standard settings). Several small molecules including the morphinan co-crystal ligand (unbound), Ibogaine, Noribogaine and Voacangine were imported as 2D SDF into Maestro and 3D structure representations were generated using LigPrep (default settings); two representations (inverted at the tertiary bridgehead nitrogen) were generated for each ligand. These were docking using Glide SP (standard settings except keeping 5 poses per compound out of 30 for post-minimization). The docked morphinan ligand reproduced the co-crystal almost perfectly. This docked complex was then optimized using Prime Refine Protein-Ligand complex (default settings). This complex was then used to generate another docking grid using Glide (default settings around the ligand) followed by Glide SP docking of the prepared ligands. In these results, the top poses of noribogaine and ibogaine aligned well the morphinan antagonist (hydrophobic Ibogaine and Noribogaine bicyclic system and ethyl substituent with morphinan cyclopropyl residues and the positively charged tertiary amines, which all form a hydrogen bond to the site chain of Asp147). The μ-OR noribogaine docking complex was then used in a 12 ns molecular dynamics (MD) simulation. The MD system generation and simulations were performed in Desmond using an all atom system with a membrane model and explicit water model (ASP). The Desmond software automatically sets up the systems (adjust charges, adds water molecules) and performs several rounds of minimization and short simulations before the 12 ns production run. MD was run on the Pegasus 2 cluster at the Center for Computational Science at the

University of Miami (http://ccs.miami.edu/hpc/) using 48 processors and completed in less than 19 hours. Simulation analysis was performed using the Desmond trajectory analysis software. A representative frame with these most prevalent interactions throughout the simulation was extracted from the trajectory, processed via protein preparation (including constrained minimization) to remove overlapping atoms, and visualized using PyMol.

Apparent binding affinities of noribogaine to OPRM and OPRK: Competitive inhibition of [³H]-U69,593 to human OPRK and of [³H]-DAMGO to human OPRM (μ-opioid receptor) by noribogaine was conducted and compared to ibogaine, 18-methoxycoronaridine (18-MC) and various control ligands (FIG. 1, Table 5). Noribogaine exhibited the highest apparent affinity for OPRK(κ-opioid receptor) with a Kᵢ value of 720±128 nM. Ibogaine displayed a Kᵢ of 3.68±0.22 μM, while 18-MC had a Kᵢ a 1.84±0.12 μM. At the OPRM, noribogaine displayed a Kᵢ of 1.52±0.3 μM, while ibogaine and 18-MC Kᵢ values were 6.92±0.83 μM and 2.26±0.35 μM respectively. Values of both noribogaine and ibogaine for the human OPRM/K receptors were comparable that of the calf OPRM and OPRK receptors (1.52 and 0.96 μM, Table 5) where noribogaine was previously shown to be ˜30× less affine at OPRD (δ-opioid receptor) than at OPRK. In assays, 18-MC was equi-affine to both human OPRK and OPRM, contrary to the reported 5× selectivity at OPRM. Experimental values, historical values from the literature, and control ligands, are displayed in Table 5 for agonists, partial agonists, and antagonists used.

As shown in Table 5, binding affinity of Noribogaine and other drugs at the human mu (OPRM) and kappa (OPRK) opioid receptors was examined. Kᵢ values of Noribogaine, Ibogaine, 18-MC (n≧3). Values for control ligands Morphine, Naloxone DAMGO, U69,593, Dynorphin A, [Met]-Enkephalin, Nalmefene, Buprenorphine were determined and/or gathered from the literature. Specificity for the OPRK receptor was evaluated using ΔpKᵢ=pKᵢ(OPRK)-pKᵢ(OPRM). Agonists (“*”), partial agonists (“̂”), and antagonists (“#”)

TABLE 5
Binding affinity of Noribogaine and other drugs at the human mu (OPRM) and kappa (OPRK) opioid receptors.

| | OPRM | | | OPRK | | | | |
|---|---|---|---|---|---|---|---|---|
| | [³H]-DAMGO binding | | | [³H]-U69,593 Binding | | | Specificity | |
| Compound | pKᵢ | Kᵢ (nM) | SEM | pKᵢ | Kᵢ (nM) | SEM | ΔpKᵢ | References |
| U69,593 | | N.Q. | | 9.2* | 0.59/0.87 | | >3 | Perkin Elmer/This work |
| DAMGO | 9.1* | 0.6/0.5 | 0.2 | | N.Q. | | <−3 | (Toll, Berzetei-Gurske et al. |
| | | | | | | | | 1998)/This work |
| Dynorphin A | 8.1* | 7.7 | 2.2 | 8.8* | 1.7-0.05** | 0.85-0.01** | 0.7 | (Toll, Berzetei-Gurske et al. |
| | | | | | | | | 1998) - (Li, Zhu et al. 1993) |
| [Met]-Enkephalin | 9.2* | 0.63 | | 6.0 | 1000 | | <−3 | (Meng, Xie et al. 1993) |
| Morphine | 9.0\hat{ } | 1.1 | 0.05 | 7.3\hat{ } | 46.9 | 4.5 | −1.6 | (Toll, Berzetei-Gurske et al. |
| | | | | | | | | 1998) |
| Nalmefene | 9.0\hat{ } | 1 | | 10\hat{ } | 0.083 | 0.0008 | 1.1 | (Bart, Schluger et al. 2005) |
| Buprenorphine | 10\hat{ } | 0.08 | 0.02 | 10^{#} | 0.11 | 0.05 | −0.1 | (Huang, Kehner et al. 2001) |
| 6′GNTI | 7.1 | 82 | 21 | 8.9\hat{ } | 1.15 | 0.39 | 1.84 | (Sharma, Jones et al. 2001) |
| Noribogaine | 5.6^{#} | 2660* | (OPRD = | 6.0\hat{ } | 960* | | 0.4 | (Pearl, Herrick-Davis et al. |
| | | | 24720) | | | | | 1995) |
| | 5.8^{#} | 1520 | 300 | 6.1\hat{ } | 720 | 128 | 0.3 | This work |
| Ibogaine | 5.0* | 11040* | (OPRD- | 5.4* | 3770 | | 0.5 | (Pearl, Herrick-Davis et al. |
| | | | N.Q.) | | | | | 1995) |
| | 5.2* | 6920 | 830 | 5.4* | 3680 | 220 | 0.3 | This work |
| 18-MC | 6.0* | 1100* | 300 | 5.3* | 5100* | 500 | −0.7 | (Glick, Maisonneuve et al. |
| | | | | | | | | 2000) |
| | 5.6* | 2360 | 350 | 5.7* | 1840 | 120 | 0.1 | This work |
| Naloxone | 8.9* | 1.4/1.3 | 0.05 | 8.6* | 2.5 | 0.3 | −0.3 | (Toll, Berzetei-Gurske et al. |
| | | | | | | | | 1998)/This work |
| NorBNI | 7.7* | 21 | 5 | 9.7* | 0.2-0.04** | 0.05-0.01** | 2.0 | (Toll, Berzetei-Gurske et al. |
| | | | | | | | | 1998) - (Li, Zhu et al. 1993) |

*calf receptor;
**[³H]diprenorphine binding; OPRD: human opioid receptor delta; N.Q. non-quantifiable

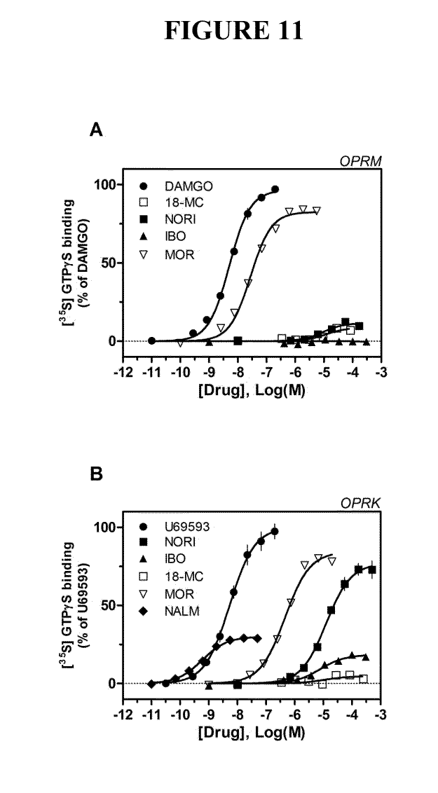

Functional agonist properties of noribogaine at OPRM and OPRK [³⁵S]GTPγS binding stimulation: [³⁵S]GTPγS binding to membranes of CHO cells stably transfected with OPRK or OPRM was examined in response to noribogaine, ibogaine, morphine, and nalmefene drug treatment and measured the activation of the G-protein pathway by agonists (FIG. 11). The prototypical full agonist U69,593, and the endogenous ultra-potent agonist, Dynorphin A, were used as controls for OPRK function and DAMGO was used for OPRM. Calculated EC₅₀ and Eₘₐₓ values are enumerated in Table 6.

Noribogaine was marginally active at stimulating [³⁵S]GTPγS binding to OPRM, with an Eₘₐₓ of 10% the full agonist DAMGO (FIG. 11A) and comparable to the level of activation previously reported. Morphine was a partial agonist with an Eₘₐₓ of 80±4.5% of DAMGO signal and an EC₅₀ of 32±1.2 nM. The partial agonist buprenorphine stimulated OPRM with an Eₘₐₓ of 26±2.2% in the assays, and ibogaine and 18-MC failed to stimulate the OPRM G-protein pathway.

Noribogaine was a partial agonist at stimulating [³⁵S]GTPγS binding to OPRK with an Eₘₐₓ of 72±3.8% of U69,593, and an EC₅₀ of 8.75±1.09 μM (FIG. 11B). Ibogaine displayed a lower agonist power than noribogaine at OPRK with an Eₘₐₓ of 18±1.4%, while 18-MC failed to stimulate [³⁵S]GTPγS binding to OPRK. In the assays, morphine and dynorphin A displayed Eₘₐₓ values of 91±7% and 94±7% respectively, and nalmefene, a partial agonist of OPRK, maximally stimulated at 35±4.7% and similar to formally reported values.

The coupling efficiencies of agonists DAMGO, U69,593, morphine, dynorphin A, nalmefene, 6′GNTI, noribogaine and ibogaine at the G-protein pathway were calculated and found to be congruent with values shifted by ˜1 log (Tables 2 and 5). Dynorphin A and 6′GNTI were outliers and displayed better coupling efficiencies (0.56 and 0.26) than other agonists at stimulating [³⁵S]GTPγS binding in comparison to their apparent binding affinities against [³H]U69,593 and [³H]diprenorphin (Tables 2 and 5).

As shown in Table 6, activation and inhibition by noribogaine of [³⁵S]GTPγS binding in CHO—K1 stably expressing human OPRM and OPRK were examined. Data used for the non-linear regression analysis are shown as the mean±SEM of (n) experiments. [Met]-Enkephalin and 6′GTNI values were gathered from references as indicated. Non-italic section indicates values (EC₅₀) for the activation component of the ligand and italic section indicates the values (Kₑ) for the inhibitory component of the ligand. Coupling efficiency (e-coupling) was calculated as in methods. Outliers are underlined.

TABLE 6
Activation and inhibition by Noribogaine of [³⁵S]GTPγS binding in CHO-K1 stably expressing human OPRM and OPRK

| | [35S]GTPgS Binding Activation | | | | | | | | |
|---|---|---|---|---|---|---|---|---|---|
| Compound | | EC₅₀ | | | Efficacy | | | | |
| Activation/Inhibition | pEC₅₀ | (nM) | (SEM) | n | (%) | (SEM) | n | e-coupling | References |
| OPRM: | | | | | | | | | |
| DAMGO | 7.5 | 29 | 9 | 7 | 100 | | n.a. | 1.77 | |
| [Met]-Enkephalin | 7.4 | ~40 | | | ~95 | | | 1.8 | (Saidak, Blake-Palmer et al. 2006) |
| Morphine | 7.5 | 32 | 1.2 | 3 | 80 | 4.5 | 4 | 1.47 | |
| Buprenorphine | | n.d. | | | 26 | 2.2 | 2 | n/d | |
| Noribogaine | 4.8 | 16050 | 9409 | 4 | 9.4 | 1.8 | 4 | 1.02 | |
| Ibogaine | | n.d. | | 2 | −2.9 | | | | |
| 18-MC | | n.d. | | 2 | <5 | | | | |
| 6′GNTI | | >1000 | | | 0~ | | | | (Waldhoer, Fong et al. 2005) |
| Noribogaine (Kₑ) | 4.7 | 19203 | 5168 | | −100~ | | | 1.10 | |
| Naloxone (Kₑ) | 8.5 | 3.36 | 0.75 | | −100 | | | 0.38 | |
| OPRK: | | | | | | | | | |
| U69,593 | 8.1 | 7.25 | 0.9 | 9 | 100 | | n.a. | 0.92 | |
| Dynorphin A | 9.7 | 0.18 | 0.04 | 6 | 94 | 7 | 3 | 0.56 | |
| Morphine | 6.4 | 434 | 67 | 4 | 91 | 7 | 3 | 0.97 | |
| Noribogaine | 5.1 | 8749 | 1092 | 10 | 72 | 3.8 | 14 | 1.08 | |
| Nalmefene | 9.2 | 0.69 | 0.14 | 3 | 35 | 4.7 | 3 | 0.92 | |
| 6′GNTI | 8.7 | 2.1 | 0.5 | | 37 | 2 | | 0.26 | (Schmid, Streicher et al. 2013) |
| Ibogaine | 4.9 | 12000~ | | 2 | 18 | 1.4 | | 1.39~ | |
| 18-MC | 4.8 | 16000~ | | 2 | <5 | | | 0.94~ | |
| Noribogaine (Kₑ)- U69 | 4.9 | 11560 | 786 | | −30 | | | 1.22 | |
| Noribogaine (Kₑ) -Dyn | 4.4 | 39797 | 15560 | | −25 | | | 1.72 | |
| Nalmefene (Kₑ) | 9.9 | 0.14 | 0.04 | | −65 | | | 0.23 | |
| 6′GNTI (Kₑ) | 9.7 | 0.18 | | | −32 | | | −0.81 | Adapted from (Schmid, |
| | | | | | | | | | Streicher et al. 2013) |
| 18-MC (Kₑ) | 5.3 | 4556 | 1392 | | −100 | | | 0.39 | |
| NorBNI (Kₑ) | 10.5 | 0.03269 | | | −100 | | | −0.1 | |
| Naloxone (Kₑ) | 8.5 | 3.36 | 0.75 | | −100 | | | 0.13 | |

n/a non-applicable.
n/d not determined

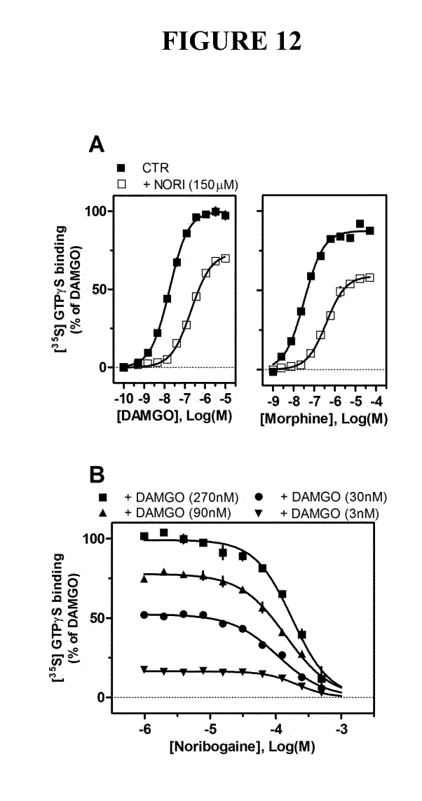

Functional inhibitory properties of noribogaine at OPRM [³⁵ S]GTPγS binding stimulation: Noribogaine marginally stimulated [³⁵S]GTPγS binding via OPRM with an approximated EC₅₀ of 16 μM (FIG. 11A). Therefore, whether noribogaine was an antagonist of OPRM was investigated. DAMGO and morphine dose responses were carried out in the presence and absence of 150 μM of noribogaine (FIG. 12A). Noribogaine was an inhibitor of both agonists tested and right-shifted their EC₅₀ by a magnitude of ˜1 log. The calculated Kₑ values (see methods) were 19±5 μM against DAMGO and 28±14 μM against morphine (Table 6) and both were in the concentration range of the EC₅₀ of noribogaine at OPRM G-protein pathway. In a similar design, naloxone displayed a Kₑ of 3.36±0.75 nM, a value close to its Kᵢ at OPRM (Table 5 and 6). Noribogaine also decreased the Eₘₐₓ of both DAMGO and morphine dose-responses curves in this assay (FIG. 12A), indicating partial unsurmountable antagonism that can encompass several distinct molecular mechanisms such as (a) irreversible competitive antagonism, (b) noncompetitive antagonism, and/or (c) functional antagonism; for review. Dose-inhibition curves of noribogaine against increasing doses of DAMGO were performed (FIG. 12B). Noribogaine dose-dependently inhibited DAMGO-stimulated [³⁵S]GTPγS binding at OPRM with an IC₅₀ of 134±17 μM independent of the agonist concentrations of DAMGO. As shown in FIG. 12, noribogaine inhibits agonist-induced [³⁵S]GTPγS binding at the mu receptor (OPRM).

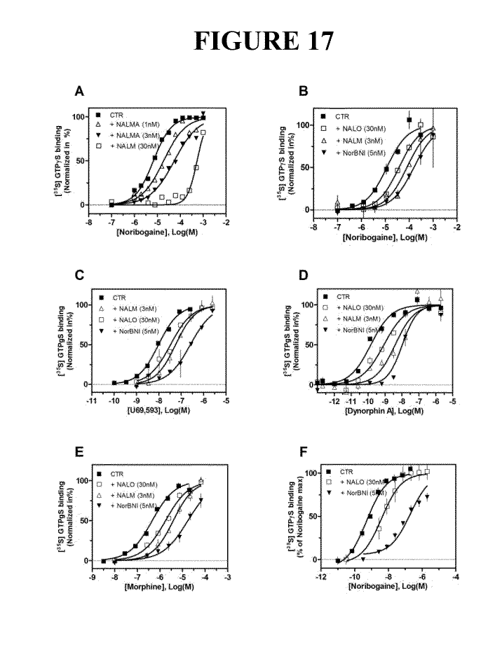

Functional inhibition of noribogaine-induced OPRK [³⁵S]GTPγS binding by antagonist: inhibitory effects of naloxone, Nor-BNI, and nalmefene on the agonist-induced [³⁵S]GTPγS binding to OPRK by noribogaine and U69,593, Dynorphin A, morphine, and nalmefene were investigated (FIG. 17, Table 7). Dose-responses to these agonists were gathered in the absence and presence of fixed antagonist concentration: 30 nM Naloxone, 5 nM Nor-BNI, and 3 nM nalmefene. The compound 18-MC was examined, which appeared to be an antagonist in this assay with a Kₑ of 4.5±1.4 μM against U69,593.

Antagonists and partial agonist nalmefene dose-dependently right-shifted the dose-response curves of noribogaine, consistent with the addition of a surmountable competitor of the noribogaine binding site (FIG. 17). Functional inhibition constants (Kₑ) for antagonists are shown in Table 7 with the assumption of ideal conditions of competitiveness and equilibrium. In all instances, the functional inhibition constants for these inhibitors were close to their Kᵢ, indicating that noribogaine was no different than other agonists tested and was apparently competing for a common binding site.

As shown in Table 7, functional inhibition constants Kₑ of Noribogaine and other ligands to agonist-induced [³⁵S]GTPγS binding in CHO—K1 stably expressing human OPRK were examined. Data used for the non-linear regression analysis are shown as the mean±SEM of 3 up to 7 experiments. Italic value represents the estimate of a hypothetical functional activation constant of designated agonist in the presence of other agonists.

TABLE 7
Functional inhibition constants Kₑ of Noribogaine and other ligands to agonist-induced
[³⁵S]GTPγS binding in CHO-K1 stably expressing human OPRK

| Antagonists & | | | | | | | |
|---|---|---|---|---|---|---|---|
| Rival agonists | Agonists | | | | | | |
| Kₑ (nM) | U69′593 | Dynorphin A | Morphine | Noribogaine | Nalmefene | Kᵢ | EC₅₀ |
| U69′593 | n/a | n/a | n/d | 0.4 | 4 | 0.9 | 7.3 |
| Dynorphin A | n/a | n/a | n/a | 0.003 | 0.1 | 0.05 | 0.18 |
| Morphine | n/d | n/a | n/a | 74 | 270 | 47 | 434 |
| Noribogaine | 12e3 ± 0.8e3 | 40e3 ± 16e3 | 15e3 ± 4e3 | n/a | 24e3 | 700 | 8.7e3 |
| Nalmefene | 0.14 ± 0.04 | 0.077 ± 0.016 | 0.11 ± 0.005 | 0.33 ± 0.07 | n/a | 0.08 | 0.7 |
| Naloxone | 8.6 ± 1.3 | 4.8 ± 0.9 | 8.2 ± 1.2 | 4.2 ± 2.3 | 9.2 | 2.5 | n/a |
| NorBNI | 0.12 ± 0.04 | 0.029 ± 0.004 | 0.07 ± 0.013 | 0.075 ± 0.036 | 0.1 ± 0.09 | 0.2 | n/a |
| 18-MC | 4.5e3 ± 1.4e3 | 2.8e3 ± 0.6e3 | 2.9e3 ± 0.7e3 | 4.3e3 ± 1.9e3 | n/d | 1.8e3 | n/a |

n/a non-applicable.
n/d not determined

Residual functional antagonist properties of noribogaine at OPRK [³⁵S]GTPγS binding stimulation: Noribogaine was a partial agonist at OPRK in the [³⁵S]GTPγS binding stimulation assays (FIG. 11). Therefore, whether noribogaine, termed here as ‘rival agonist’, and the partial agonists nalmefene was able to functionally compete with and level down the activity of more efficacious agonists than itself was determined.

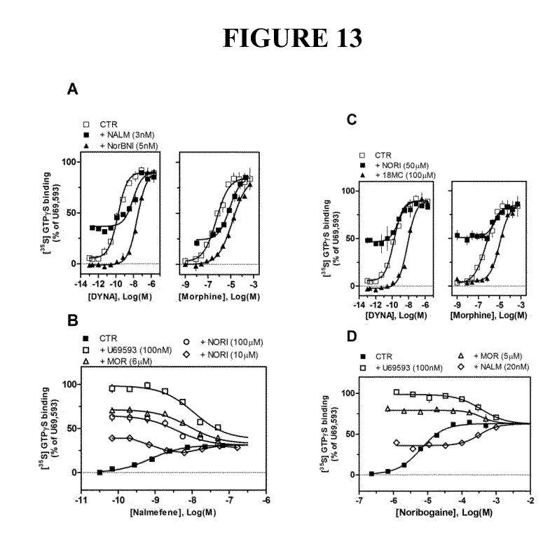

Dynorphin A and morphine dose-responses curves were performed in the presence and the absence of rival agonists nalmefene or noribogaine at concentrations of˜5× their EC₅₀ (nalmefene 3 nM, noribogaine 50 μM) (FIG. 13A, 13C). Nalmefene readily right-shifted the EC₅₀ of Dynorphin A and morphine with an apparent Kₑ of 0.077±0.016 nM and 0.11±0.005 nM, within the range of its apparent Ki (0.08 nM) and similar to the pure antagonist NorBNI (nor-binaltorphimine) (Table 7). Noribogaine, on the other hand, poorly right-shifted the EC₅₀ of these agonists and the Kₑ estimates in these conditions were 40±16 μM and 15±4 μM respectively, about 40× its (Table 7, underlined values).

In another set of experiments (FIG. 13B, 13D), noribogaine and nalmefene dose-response curves were produced in the presence of a set concentration of agonists. Nalmefene readily leveled-down the signal of moderate to high concentrations of rival full (U69,593) or partial (noribogaine) agonists to its own reduced levels (30%) with an apparent IC₅₀ proportional to the rival agonist concentration (including noribogaine). Noribogaine also leveled-down the signal of high concentrations of rival agonists to its own reduced signal (70%), but the IC₅₀ values were high (100-300 μM range). Finally, the apparent functional potency of dynorphin A, U69,593 and morphine were estimated by being set as rival agonist dose-responses in the presence of either noribogaine or nalmefene. In this setting, the calculated Kₑ (activation) for all rival agonists were lower in the presence of noribogaine than in the presence of nalmefene (close to their experimental EC₅₀) and showed that noribogaine was a somewhat poor functional blocker of these agonists (Table 7, underlined values). As shown in FIG. 13, noribogaine partially inhibits of agonist-induced [35S]GTPγS binding at the kappa receptor (OPRK).

Functional antagonist properties of noribogaine at OPRK-mediated β-arrestin-2 recruitment: PathHunter β-Arrestin GPCR assays detecting the interaction of β-Arrestin with the activated receptor were used to measure non-G protein OPRM and OPRK activity. Dose-response curves to noribogaine were compared to full agonists [Met]-enkephalin (OPRM), and Dynorphin A (OPRK) drug treatments

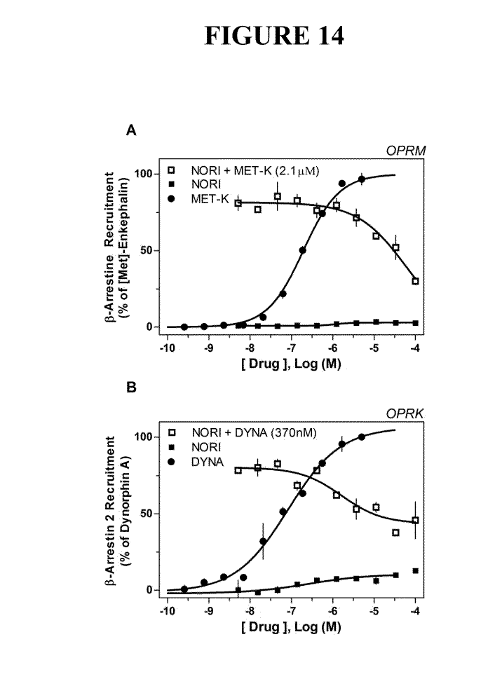

(FIGS. 14A and 14B). Calculated EC₅₀ values, maximal responses and coupling efficiencies are shown in Table 8. Control agonist [Met]-Enkephalin displayed an EC₅₀ of 193±11 nM at OPRM and Dynorphin A displayed an EC₅₀ of 82±21 nM at OPRK. Noribogaine displayed a profound functional bias at OPRK and was marginally efficacious at inducing β-Arrestin-2 recruitment at OPRK with an Eₘₐₓ of 12.6±3% of dynorphin A maximal stimulation and an estimated EC₅₀ of 265 nM. Noribogaine was also not an agonist at OPRM.

Noribogaine was then tested for its ability to inhibit agonist-induced β-Arrestin-2 recruitment at OPRM and OPRK (FIG. 14A, 14B). In these assays, β-Arrestin-2 recruitment was induced by the agonists [Met]-Enkephalin (OPRM) and Dynorphin A (OPRK) at their EC₈₀ concentration and challenged with increasing concentrations of noribogaine. Noribogaine inhibited agonist responses at OPRM and OPRK up to 60-100% and ˜60%, with an IC₅₀ of ˜57 μM and 1.45±1.1 μM respectively (FIG. 14). The Kₑ values were ˜4.8 μM and 262 nM for OPRM and OPRK respectively (Table 8). Thus, noribogaine was apparently 144× more potent at inhibiting Dynorphin A-induced β-arrestin-2 recruitment than at inhibiting Dynorphin A-induced G-protein activation (Table 9). As shown in FIG. 14, noribogaine inhibits agonist-induced β-arrestin recruitment at the mu (OPRM) and kappa (OPRK) receptors.

As shown in Table 8, activation and inhibition by Noribogaine of β-arrestin 2 recruitment in CHO—K1 stably expressing human OPRM and OPRK were examined. Data used for the non-linear regression analysis are shown as the mean±SEM of one standardized experiment. Morphine, buprenorphine and 6′GTNI values were gathered from references as indicated. Non-italic section indicates values (EC₅₀) for the activation component of the ligand and italic section indicates the values (Kₑ) for the inhibitory component of the ligand. Coupling efficiency (e-coupling) was calculated as in methods.

TABLE 8
Activation and inhibition by Noribogaine of β-arrestin
2 recruitment in CHO-K1 stably expressing human OPRM and OPRK

| | β-arrestin 2 recruitment | | | | | | |
|---|---|---|---|---|---|---|---|
| Compound | | EC₅₀ | | Efficacy | | | |
| Activation/Inhibition | pEC₅₀ | (nM) | (SEM) | (%) | (SEM) | e-coupling | References |
| OPRM: | | | | | | | |
| DAMGO | 6.1 | 794 | | 100 | | 3.20 | (DeWire, Yamashita et al. 2013) |
| [Met]-Enkephalin | 6.7 | 193 | 11 | 100 | | 1.94 | |
| Morphine | 6.3 | 501 | | 11.3 | | 2.66 | (DeWire, Yamashita et al. 2013) |
| Buprenorphine | | n/a | | 0 | | n/a | (DeWire, Yamashita et al. 2013) |
| Noribogaine | 5.9 | ~1150 | | 3 | 0.5 | n/a | |
| Noribogaine (Kₑ) | 4.2 | ~4794 | | −100~ | | ~1.10 | |
| OPRK: | | | | | | | |
| U69′593 | 7.2 | 59 | | 100 | | 1.83 | (Schmid, Streicher et al. 2013) |
| Dynorphin A | 7.1 | 82 | 21 | 100 | | 3.22 | |
| Noribogaine | 6.6 | ~265 | | 12.6 | 3 | −0.43 | |
| 6′GNTI | 8.2 | 5.9 | 3.3 | 12 | 3 | 0.71 | (Schmid, Streicher et al. 2013) |
| Noribogaine (Kₑ)-Dyn | 6.6 | 262 | | −60~ | | −0.44 | |
| 6′GNTI (Kₑ) | 9.3 | 0.56 | | −69 | 2 | −0.31 | Adapted from (Schmid, |
| | | | | | | | Streicher et al. 2013) |
| NorBNI (Kₑ) | 9.4 | 0.37 | | −100 | | 0.27 | |

TABLE 9
Noribogaine activation and inhibition bias quantification at OPRK.

| | G-protein Pathway | | Beta-Arrestin2 Pathway | | Bias G-protein/Beta-arrestin2 | |
|---|---|---|---|---|---|---|
| | e-coupling | e-signal | e-coupling | e-signal | Bias-coupling | Bias-efficacy |
| Activation | | | | | | |
| U69′593 | 0.92 | 1.00 | 1.83 | 1.00 | 1/8 | 0 |
| Dynorphin A | 0.56 | 0.94 | 3.22 | 1.00 | 1/457 | −0.06 |
| Noribogaine | 1.08 | 0.72 | −0.43 | 0.13 | 32 | 0.59 |
| 6′GTNI | 0.26 | 0.37 | 0.71 | 0.12 | 1/3 | 0.25 |
| Inhibition | | | | | | |
| Noribogaine | 1.72 | 0.25 | −0.44 | 0.60 | 144 | −0.35 |
| 6′GTNI | −0.81 | 0.32 | −0.31 | 0.69 | 1/3 | −0.37 |

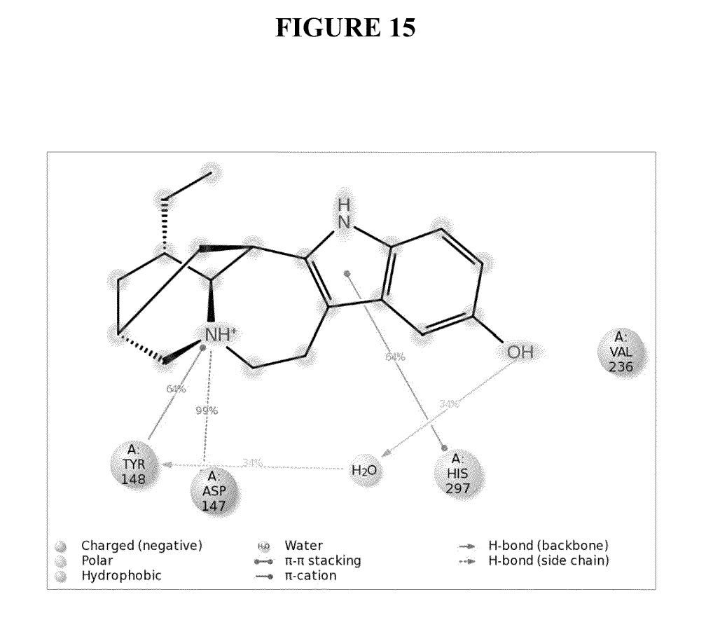

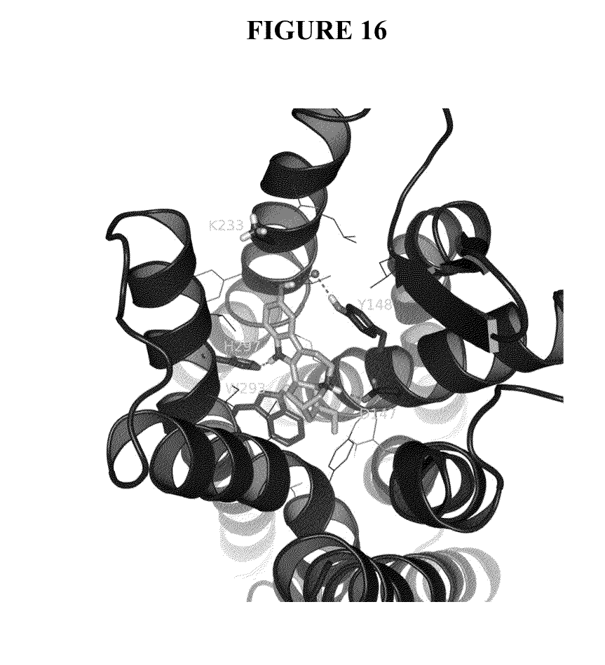

Binding Model of noribogaine and ibogaine to the inactive conformation of OPRM: An in silico binding model was developed based on the mouse OPRM co-crystal structure [PMID 22437502] as described in methods. The mouse and human OPRM share 94% (global) sequence identity and all binding site residues are identical. After initial optimization of the model, the top docking poses of noribogaine and ibogaine were pharmacophorically aligned with the co-crystal morphinan antagonist: the hydrophobic ibogaine and noribogaine bicyclic system and ethyl substituent with morphinan cyclopropyl residues were spatially aligned and the positively charged tertiary amines were superimposed with each forming a hydrogen bond to the site chain of Asp147. Then, the noribogaine and ibogaine OPRM complexes were each used in a 12 ns all atom explicit water molecular dynamics simulation. Trajectory analysis revealed the most prevalent interactions of noribogaine (FIG. 15) and ibogaine. Both ligands formed a stable hydrogen bond with Asp147 via their tertiary amine Noribogaine and ibogaine formed pi-cation interaction with Tyr148 (64 and 56%, respectively), and hydrophobic interactions with His297 (64 and 93%, respectively). Further hydrophobic interactions were observed between Val236 (˜40 and ˜60%, respectively), Tyr326 (˜20 and ˜40% respectively), Met151 (˜20% and ˜30%, respectively) and also Trp293, Ile296, Val300. Characteristically, noribogaine, but not ibogaine, formed a water bridge with Tyr148 for 34% of the simulation time. Both ligands showed a hydrogen bond with His297 for about 20% of the simulation. Movies of the simulations were generated and are available as supporting material. A representative illustration frame of noribogaine in the OPRM was extracted from the simulation and is shown in FIG. 16. As shown in FIG. 17, inhibition of Noribogaine induced [35S]GTPγS binding by OPRK antagonists.

In some embodiments, noribogaine is shown to be a dual ligand of both mu and kappa opioid receptors (OPRM and OPRK) with peculiar pharmacological properties. In some embodiments, noribogaine displayed mixed agonism-antagonism properties and a profound G-protein bias at the opioid receptors. In some embodiments, noribogaine also incurred functional selectivity to the otherwise unbiased dynorphin A signaling and the kappa system.

Noribogaine is the primary metabolite of the anti-addictive substance ibogaine, which modulates opiate analgesic activity and the components of drug addiction in animal models at brain concentrations of 0.5-15 μM. Noribogaine was a moderately potent antagonist of the OPRM G-protein and β-arrestin signaling pathways (20 μM; 48 μM). Noribogaine was a partial agonist at the OPRK G-protein pathway and activated at 75% the maximal efficacy of Dynorphin A (Dyn-A), a potency of 9 μM and weak inhibitory properties (40 μM, 25% against Dyn-A). Noribogaine was a biased agonist and poorly activated the OPRK β-arrestin pathway at 12% of Dyn-A maximal efficacy. Noribogaine was able to functionally inhibit Dyn-A-induced β-arrestin recruitment (Dyn-A EC₅₀: 82 nM) at physiologically relevant concentrations (IC₅₀ of 1.45 μM at 370 nM Dyn-A). Computational simulations indicated that noribogaine may bind to the orthosteric morphinan binding site of the receptor.

Noribogaine is demonstrated to be a mixed agonist-antagonist of OPRK and OPRM as well as a profound G-protein biased OPRK ligand. Ibogaine and 18-MC were either not agonists or poor agonists of OPRM or OPRK. 18-MC was a regular competitive antagonist of agonist-induced OPRK signaling. Noribogaine may belong to a different class of opioid ligands than ibogaine or 18-MC.

What is claimed is:

1. A method for modulating tolerance to an opioid analgesic in a patient undergoing opioid analgesic therapy, the method comprising interrupting or administering concurrently with said opioid analgesic therapy an amount of noribogaine, noribogaine derivative, or pharmaceutically acceptable salt and/or solvate thereof that provides an average serum concentration of 50 ng/mL to 180 ng/mL, said concentration being sufficient to re-sensitize the patient to the opioid as an analgesic while maintaining a QT interval of less than about 500 ms during said treatment.

2. The method of claim 1, wherein the noribogaine, noribogaine derivative, or pharmaceutically acceptable salt and/or solvate thereof is administered as a single dose or multiple doses.

3. The method of claim 1, further comprising interrupting the dosage of the analgesic.

4. The method of claim 1, further comprising administering noribogaine, noribogaine derivative, or pharmaceutically acceptable salt and/or solvate thereof concurrently with the analgesic.

5. The method of claim 4, wherein during concurrent administration, the dose of opioid analgesic is reduced.

6. The method of claim 2, comprising:
a) administering an initial dose of noribogaine, noribogaine derivative, or pharmaceutically acceptable salt or solvate thereof, wherein the initial dose provides an average serum concentration of 50 ng/mL to 180 ng/mL; and
b) administering at least one additional dose of noribogaine, noribogaine derivative, or pharmaceutically acceptable salt or solvate thereof, such that the at least one additional dose maintains the average serum concentration of 50 ng/mL to 180 ng/mL for a period of time.

7. The method of claim 6, wherein the initial dose is from 75 mg to 120 mg.

8. The method of claim 6, wherein the at least one additional dose is from 5 mg to 25 mg.

9. The method of claim 6, wherein the at least one additional dose is administered from 6 hours to 24 hours after the initial dose.

10. The method of claim 6, wherein at least two additional doses are administered, and further wherein the additional doses are administered from 6 hours to 24 hours after the previous dose.

11. The method of claim 1, further comprising selecting an addicted patient who is prescreened to evaluate tolerance for prolongation of QT interval.

12. A method for modulating tolerance to an opioid analgesic in a patient undergoing opioid analgesic therapy, the method comprising interrupting or administering concurrently with said opioid analgesic therapy an amount of noribogaine, noribogaine derivative, or pharmaceutically acceptable salt and/or solvate thereof that provides an average serum concentration of 50 ng/mL to 180 ng/mL, said concentration being sufficient to re-sensitize the patient to the opioid as an analgesic while maintaining a QT interval prolongation of less than about 20 ms during said treatment.

13. The method of claim 12, wherein the noribogaine, noribogaine derivative, or pharmaceutically acceptable salt and/or solvate thereof is administered as a single dose or multiple doses.

14. The method of claim 12, further comprising interrupting the dosage of the analgesic.

15. The method of claim 12, further comprising administering noribogaine, noribogaine derivative, or pharmaceutically acceptable salt and/or solvate thereof concurrently with the analgesic.

16. The method of claim 15, wherein during concurrent administration, the dose of opioid analgesic is reduced.

17. The method of claim 12, further comprising:
a) administering an initial dose of noribogaine, noribogaine derivative, or pharmaceutically acceptable salt or solvate thereof, wherein the initial dose provides an average serum concentration of 50 ng/mL to 180 ng/mL; and
b) administering at least one additional dose of noribogaine, noribogaine derivative, or pharmaceutically acceptable salt or solvate thereof, such that the at least one additional dose maintains the average serum concentration of 50 ng/mL to 180 ng/mL for a period of time.

18. The method of claim 17, wherein the initial dose is from 75 mg to 120 mg.

19. The method of claim 17, wherein the at least one additional dose is from 5 mg to 25 mg.

20. The method of claim 17, wherein the at least one additional dose is administered from 6 hours to 24 hours after the initial dose.

21. The method of claim 17, wherein at least two additional doses are administered, and further wherein the additional doses are administered from 6 hours to 24 hours after the previous dose.

22. The method of claim 12, further comprising selecting an addicted patient who is prescreened to evaluate tolerance for prolongation of QT interval.

23. The method of claim 1, wherein the opioid analgesic is selected from the group consisting of fentanyl, hydrocodone, hydromorphone, morphine, oxycodone, buprenorphine, codeine, thebaine, buprenorphine, methadone, meperidine, tramadol, tapentadol, levorphanol, sufentanil, pentazocine, and oxymorphone.

24. The method of claim 23, wherein the opioid analgesic is morphine.
